(19) United States
(12) Patent Application Publication
(10) Pub. No.: US 2005/0019801 A1
(43) Pub. Date: Jan. 27, 2005

(54) Stem cell-based methods for identifying and characterizing agents

(75) Inventors: Lee L. Rubin, Wellesley, MA (US); Karen Kotkow, Jamaica Plain, MA (US); Cheng-Jung Lai, Belmont, MA (US)

Correspondence Address: ROPES & GRAY LLP, ONE INTERNATIONAL PLACE, BOSTON, MA, 02110-2624, US

(73) Assignee: Curis, Inc., Cambridge, MA (US)

(21) Appl. No.: 10/861,040

(22) Filed: Jun. 4, 2004

Related U.S. Application Data
(60) Provisional application No. 60/476,011, filed on Jun. 4, 2003.

(51) Int. Cl. C12Q 1/68; G01N 33/53; G01N 33/567; C12N 5/08

(52) U.S. Cl. 435/6; 435/7.2; 435/366

(57) ABSTRACT

The present invention provides methods of identifying and/or characterizing agents that promote differentiation of stem cells to a particular differentiated cell type. The invention further provides methods of treating injuries and degenerative diseases by administering agents that promote the differentiation of stem cells to particular differentiated cell types.

### RELATED APPLICATIONS

This application claims priority to U.S. provisional application 60/476,011, filed Jun. 4, 2003, the disclosure of which is hereby incorporated by reference in its entirety.

### BACKGROUND OF THE INVENTION

The past several years has seen a flurry of activity directed to the identification and characterization of stem cells derived from various embryonic, fetal, and adult tissue sources. Stem cells isolated from these sources have shown the ability to give rise, under particular conditions, to various differentiated cell types. This body of work has fueled tremendous interest in the possibility that stem cell-based therapies represent a treatment option for a wide range of conditions affecting virtually any tissue in the body.

However, several impediments hamper the realization of stem cell-based therapies. Firstly, many have raised ethical as well as safety concerns regarding the use of human tissue as part of a therapeutic regimen. Secondly, many protocols that aim to direct the differentiation of stem cells to particular desired cell types are woefully inefficient. These shortcomings must be addressed in order to facilitate the leap from stem cells in the laboratory to stem cells in the clinic.

One way in which to bridge this divide is through the identification and characterization of agents that promote the efficient differentiation of stem cells to a particular desired cell type. Without being bound by theory, agents that promote differentiation of stem cell to a particular differentiated cell types may be useful for administration to a patient. Given the currently accepted theory that stem cells resident in tissues can be mobilized to help repair cellular damage, such agents could be used to stimulate endogenous stem cells to differentiate to particular lineages and thus alleviate the need to use cellular based therapeutics. Accordingly, the present invention provides methods of identifying and characterizing agents that promote the differentiation of stem cells to particular differentiated cell types, as well as the use of agents identified by these methods in the treatment of injuries and diseases.

### SUMMARY OF THE INVENTION

Stem cells, with their capacity to differentiate into any of a number of cell types, hold great promise both for therapeutic purposes and as a resource for exploring questions in basic developmental biology. However, the tremendous developmental potential of many stem cells has also hampered their use as a therapeutic since any therapeutic will likely require the controlled differentiation of stem cells to particular developmental fates. Accordingly, the present invention provides methods to identify agents capable of promoting differentiation of stem cells to particular cell types. The invention contemplates both the identification of agents that promote terminal differentiation, as well as agents that promote the progressive differentiation of cells from stem cells to cells of increasing commitment along a particular developmental fate. Additionally, the invention contemplates the identification of agents that promote the differentiation (terminal differentiation or progressive differentiation) of cells that are not stem cells (i.e., cells that already have been biased to some degree to differentiate along a particular lineage). The invention still further contemplates that agents identified by these methods may be useful in a therapeutic context to either influence the fate of endogenous cells, or to influence the fate of cells engineered ex vivo.

In a first aspect, the present invention provides a method for identifying and/or characterizing one or more agents that promote the differentiation of a stem cell to a particular differentiated cell type. The method comprises the following steps: providing a culture comprising stem cells, contacting the culture with one or more factors which help to bias the stem cell down a particular developmental lineage (i.e., ectodermal, mesodermal, endodermal), contacting said biased culture of cells with one or more test agents, and detecting the expression of one or more markers which identify the differentiation of a stem cell to a particular differentiated cell type. In the foregoing method, the one or more agents that promote expression of one or more markers of said particular differentiated cell type are identified as agents that promote the differentiation of a stem cell to a particular differentiated cell type.

In one embodiment, the stem cells are derived from a mammal. In another embodiment, the stem cells are derived from any of mice, rats, cows, pigs, humans, or non-human primates.

In one embodiment, the stem cells are selected from embryonic stem cells or adult stem cells. In another embodiment, the adult stem cells are selected from the group consisting of mesenchymal stem cells, neural stem cells, neural crest stem cells, hematopoietic stem cells, and pancreatic stem cells.

In one embodiment, the one or more factors that bias the stem cells along a developmental lineage are independently selected from the group consisting of nucleic acids, peptides, polypeptides, small organic molecules, antibodies, ribozymes, antisense oligonucleotides, and RNAi constructs. In another embodiment, the one or more test agents are independently selected from the group consisting of nucleic acids, peptides, polypeptides, small organic molecules, antibodies, ribozymes, antisense oligonucleotides, and RNAi constructs.

In another embodiment, the one or more agents is a library of agents.

In another embodiment, the particular differentiated cell type is a neuronal cell type. In yet another embodiment, the neuronal cell type is selected from the group consisting of motor neurons, dopaminergic neurons, cholinergic neurons, interneurons, sensory neurons, serotonergic neurons, peptodergic neurons, astrocytes, and oligodendrocytes.

In another embodiment, the factor that biases stem cells to differentiate along a particular developmental lineage biases cells to an ectodermal, mesodermal or endodermal lineage. In another embodiment, the factor that biases stem cells to differentiate along a particular developmental lineage biases cells to a neuronal lineage.

In still another embodiment, the factor that biases stem cells is retinoic acid.

In a second aspect, the present invention provides a method for identifying and/or characterizing one or more agents that promote the differentiation of an embryonic stem cell to a particular differentiated cell type. The method comprises the following steps: providing a culture comprising stem cells, contacting the culture with one or more factors which help to bias the stem cell down a particular developmental lineage (i.e., ectodermal, mesodermal, endodermal), contacting said biased culture of cells with one or more test agents, and detecting the expression of one or more markers which identify the differentiation of an embryonic stem cell to a particular differentiated cell type. In the foregoing method, the one or more agents that promote expression of one or more markers of said particular differentiated cell type are identified as agents that promote the differentiation of an embryonic stem cell to a particular differentiated cell type.

In one embodiment, the stem cells are derived from a mammal. In another embodiment, the stem cells are derived from any of mice, rats, cows, pigs, humans, or non-human primates.

In one embodiment, the one or more factors that bias the stem cells along a developmental lineage are independently selected from the group consisting of nucleic acids, peptides, polypeptides, small organic molecules, antibodies, ribozymes, antisense oligonucleotides, and RNAi constructs. In another embodiment, the one or more test agents are independently selected from the group consisting of nucleic acids, peptides, polypeptides, small organic molecules, antibodies, ribozymes, antisense oligonucleotides, and RNAi constructs.

In another embodiment, the one or more agents is a library of agents.

In another embodiment, the particular differentiated cell type is a neuronal cell type. In yet another embodiment, the neuronal cell type is selected from the group consisting of motor neurons, dopaminergic neurons, cholinergic neurons, interneurons, sensory neurons, serotonergic neurons, peptodergic neurons, astrocytes, and oligodendrocytes.

In another embodiment, the factor that biases stem cells to differentiate along a particular developmental lineage biases cells to an ectodermal, mesodermal or endodermal lineage. In another embodiment, the factor that biases stem cells to differentiate along a particular developmental lineage biases cells to a neuronal lineage.

In still another embodiment, the factor that biases stem cells is retinoic acid.

In a third aspect, the present invention provides a method for identifying and/or characterizing one or more agents that promote the differentiation of a stem cell to a differentiated neuronal cell type. The method comprises the following steps: providing a culture comprising stem cells, contacting the culture with a composition comprising retinoic acid to bias the stem cells along a neuronal lineage, contacting said biased culture of cells with one or more test agents, and detecting the expression of one or more markers which identify the differentiation of a stem cell to a particular differentiated neuronal cell type. In the foregoing method, the one or more agents that promote expression of one or more markers of said particular differentiated cell type are identified as agents that promote the differentiation of a stem cell to a particular differentiated neuronal cell type.

In one embodiment, the stem cells are derived from a mammal. In another embodiment, the stem cells are derived from any of mice, rats, cows, pigs, humans, or non-human primates.

In one embodiment, the stem cells are selected from embryonic stem cells or adult stem cells. In another embodiment, the adult stem cells are selected from the group consisting of mesenchymal stem cells, neural stem cells, neural crest stem cells, hematopoietic stem cells, and pancreatic stem cells.

In one embodiment, the one or more test agents are independently selected from the group consisting of nucleic acids, peptides, polypeptides, small organic molecules, antibodies, ribozymes, antisense oligonucleotides, and RNAi constructs.

In another embodiment, the one or more agents is a library of agents.

In another embodiment, the particular differentiated cell type is a neuronal cell type. In yet another embodiment, the neuronal cell type is selected from the group consisting of motor neurons, dopaminergic neurons, cholinergic neurons, interneurons, sensory neurons, serotonergic neurons, peptodergic neurons, astrocytes, and oligodendrocytes.

In a fourth aspect, the present invention provides a method for identifying and/or characterizing one or more agents that promote the differentiation of an embryonic stem cell to a differentiated neuronal cell type. The method comprises the following steps: providing a culture comprising embryonic stem cells, contacting the culture with a composition comprising retinoic acid to bias the embryonic stem cells along a neuronal lineage, contacting said biased culture of cells with one or more test agents, and detecting the expression of one or more markers which identify the differentiation of a stem cell to a particular differentiated neuronal cell type. In the foregoing method, the one or more agents that promote expression of one or more markers of said particular differentiated cell type are identified as agents that promote the differentiation of an embryonic stem cell to a particular differentiated neuronal cell type.

In one embodiment, the embryonic stem cells are derived from a mammal. In another embodiment, the stem cells are derived from any of mice, rats, cows, pigs, humans, or non-human primates.

In one embodiment, the one or more test agents are independently selected from the group consisting of nucleic acids, peptides, polypeptides, small organic molecules, antibodies, ribozymes, antisense oligonucleotides, and RNAi constructs.

In another embodiment, the one or more agents is a library of agents.

In another embodiment, the particular differentiated cell type is a neuronal cell type. In yet another embodiment, the neuronal cell type is selected from the group consisting of motor neurons, dopaminergic neurons, cholinergic neurons, interneurons, sensory neurons, serotonergic neurons, peptodergic neurons, astrocytes, and oligodendrocytes.

In a fifth aspect, the present invention provides a step-wise method for identifying and/or characterizing one or more agents that promote the progressive differentiation of a cell to a particular differentiated cell type. The method comprises the following steps: providing a culture comprising cells, contacting said culture of cells with one or more test agents, and detecting the expression of one or more markers which identify the progressive differentiation of a cell to a particular differentiated cell type. In the foregoing method, the one or more agents that promote expression of one or more markers of progressive differentiation of a cell to a particular differentiated cell type are identified as agents that promote the commitment of a cell to a particular differentiated cell type.

The invention contemplates that the starting cell (the input cell) can be a stem cell in which case the method comprises the identification of agents that promote the progressive differentiation of a stem cell to a cell of increasing commitment to a particular cell fate and finally to a terminally differentiated cell type. The invention similarly contemplates that the starting cell can be a cell that is not a stem cell and thus already has some degree of commitment along a particular developmental fate.

In one embodiment, the cells are derived from a mammal. In another embodiment, the cells are derived from any of mice, rats, cows, pigs, humans, or non-human primates.

In one embodiment, the cells are stem cells selected from embryonic stem cells or adult stem cells. In another embodiment, the adult stem cells are selected from the group consisting of mesenchymal stem cells, neural stem cells, neural crest stem cells, hematopoietic stem cells, and pancreatic stem cells.

In one embodiment, the one or more test agents are independently selected from the group consisting of nucleic acids, peptides, polypeptides, small organic molecules, antibodies, ribozymes, antisense oligonucleotides, and RNAi constructs.

In another embodiment, the one or more agents is a library of agents.

In another embodiment, the particular differentiated cell type is a neuronal cell type. In yet another embodiment, the neuronal cell type is selected from the group consisting of motor neurons, dopaminergic neurons, cholinergic neurons, interneurons, sensory neurons, serotonergic neurons, peptodergic neurons, astrocytes, and oligodendrocytes.

In a sixth aspect, the invention provides a pharmaceutical preparation comprising the one or more agents identified by the methods of the present invention. The pharmaceutical preparation comprises the one or more agents and a pharmaceutically acceptable carrier or excipients.

In a seventh aspect, the invention provides the use of the one or more agents identified by the methods of the present invention in the manufacture of a medicament for differentiating cells. In one embodiment, the cells are stem cells. In another embodiment, the stem cells are selected from embryonic stem cells or adult stem cells. In another embodiment, the cells are not stem cells. Though not terminally differentiated, such non-stem cells are already biased (to some degree) to differentiate along a particular developmental pathway (i.e., the cell has some level of commitment).

In an eighth aspect, the invention provides the use of the one or more agents identified by the methods of the present invention in the manufacture of a medicament for the treatment of an injury or disease.

In one embodiment, the injury or disease is of the central nervous system or the peripheral nervous system. In one embodiment, the injury or disease is selected from the group consisting of Parkinson's disease, Alzheimer's disease, Huntington's disease, ALS, multiple sclerosis, peripheral neuropathy, spinal cord injury, brain injury, macular degeneration, detached retina, and stroke. In another embodiment, the injury is a result of physical trauma, bacterial infection, viral infection, ischemia, hypoxia, or a proliferative disorder.

In another embodiment, the injury or disease is of a mesodermal tissue or endodermal tissue. Exemplary injuries and degenerative conditions of tissues derived from the mesoderm or endoderm include degenerative heart and vascular diseases such as atherosclerosis and occlusive vascular disease, degenerative conditions of cartilage and connective tissue such as osteoarthritis and rheumatoid arthritis, degenerative conditions of the liver such as cirrohis, degenerative conditions of the kidney such as polycystic kidney disease, degenerative conditions of the pancreas such as diabetes, and degenerative conditions of the digestive system including Inflammatory Bowel disease. Additionally, cancer, of any tissue, can be thought of as both a degenerative disease and as an injury. Tissue is often damaged by a combination of the effects of: progression of the disease; treatment regimens including medication, radiation therapy, and chemotherapy; and scarring and other damage caused by surgical intervention. In another embodiment, the injury is a result of physical trauma, bacterial infection, viral infection, ischemia, hypoxia, or a proliferative disorder.

In a ninth aspect, the present invention provides the use of the one or more agents identified by the methods of the present invention in the manufacture of a medicament for modulating cell proliferation and/or differentiation in vitro or in vivo.

In one embodiment, the agents are used to modulate ectodermal differentiation such as neuronal, skin, or hair follicle differentiation. In another embodiment, the agents are used to modulate mesodermal differentiation. In still another embodiment, the agents are used to modulate endodermal differentiation. In yet another embodiment, the agents are used to modulate angiogenesis.

In another embodiment, agents used to modulate ectodermal, mesodermal, or endodermal differentiation can be used therapeutically in treating a condition in a patient in need thereof.

In this and other aspects of the present invention, the invention recognizes that certain agents useful for modulating cellular proliferation and/or differentiation do so by modulating signaling via a particular signal transduction pathway. Agents that modulate signaling transduction via particular signal transduction pathways have particular in vitro uses in promoting proliferation and/or differentiation of cells, and additionally have therapeutic uses in promoting proliferation and/or differentiation of particular cells in vivo.

In one embodiment, the agent agonizes a particular signal transduction pathway. In another embodiment, the agent antagonizes a particular signal transduction pathway.

In one embodiment, the agent agonizes hedgehog signal transduction. In another embodiment, the agent antagonizes hedgehog signal transduction. In another embodiment, the agent agonizes BMP signal transduction. In another embodiment, the agent antagonizes BMP signal transduction. In one embodiment, the agent agonizes Wnt signal transduction. In another embodiment, the agent antagonizes Wnt signal transduction. In still another embodiment, the agent agonizes Notch signal transduction. In yet another embodiment, the agent antagonizes Notch signal transduction.

In a tenth aspect, the present invention provides a method of conducting a stem cell business. The method comprises identifying and/or characterizing one or more agents that promote the differentiation of stem cells to a particular differentiated cell type according to any of the screening methods of the present invention, and licensing the right to further develop these agents to a third party.

In an eleventh aspect, the present invention provides a method of conducting a stem cell business. The method comprises the following steps: identifying and/or characterizing one or more agents that promote differentiation of stem cells to a particular differentiated cell type according to the methods of the present invention, conducting therapeutic profiling of an agent so identified for safety and toxicity in one or more animal models, and formulating a pharmaceutical preparation including one or more agents which demonstrated an acceptable therapeutic profile.

In one embodiment, the method further includes establishing a system for distributing the pharmaceutical preparation for sale. In another embodiment, the method includes establishing a sales group for marketing the pharmaceutical preparation.

In a twelfth aspect, the present invention provides a method of manufacturing a compound, wherein the compound is an agent that promotes differentiation of a stem cell to a particular differentiated cell type. The method comprises the following steps: providing a culture comprising stem cells, contacting said culture with one or more factors that bias the stem cells to differentiate along a particular developmental lineage, contacting said culture with one or more test agents, detecting expression of one or more markers which identify the differentiation of a stem cell to said particular differentiated cell type. The one or more agents that promote expression of one or more markers of said particular differentiated cell type are identified as agents that promote the differentiation of a stem cell to a particular differentiated cell type. The method further includes the step of synthesizing said compound so identified as an agent that promotes differentiation of a stem cell to a particular differentiated cell type.

In one embodiment, the method further comprises formulating said compound in a pharmaceutically acceptable carrier.

In a thirteenth aspect, the present invention provides a method of manufacturing a compound, wherein the compound is an agent that promotes differentiation of an embryonic stem cell to a particular differentiated cell type. The method comprises the following steps: providing a culture comprising embryonic stem cells, contacting said culture with one or more factors that bias the stem cells to differentiate along a particular developmental lineage, contacting said culture with one or more test agents, detecting expression of one or more markers which identify the differentiation of a stem cell to said particular differentiated cell type. The one or more agents that promote expression of one or more markers of said particular differentiated cell type are identified as agents that promote the differentiation of an embryonic stem cell to a particular differentiated cell type. The method further includes the step of synthesizing said compound so identified as an agent that promotes differentiation of an embryonic stem cell to a particular differentiated cell type.

In one embodiment, the method further comprises formulating said compound in a pharmaceutically acceptable carrier.

In a fourteenth aspect, the present invention provides a method of manufacturing a compound, wherein the compound is an agent that promotes differentiation of a stem cell to a particular differentiated neuronal cell type. The method comprises the following steps: providing a culture comprising stem cells, contacting said culture with retinoic acid to bias the stem cells to differentiate along a neuronal lineage, contacting said culture with one or more test agents, detecting expression of one or more markers which identify the differentiation of a stem cell to said particular differentiated neuronal cell type. The one or more agents that promote expression of one or more markers of said particular differentiated cell type are identified as agents that promote the differentiation of a stem cell to a particular differentiated neuronal cell type. The method further includes the step of synthesizing said compound so identified as an agent that promotes differentiation of a stem cell to a particular differentiated neuronal cell type.

In one embodiment, the method further comprises formulating said compound in a pharmaceutically acceptable carrier.

In a fifteenth aspect, the present invention provides a method of manufacturing a compound, wherein the compound is an agent that promotes differentiation of an embryonic stem cell to a particular differentiated neuronal cell type. The method comprises the following steps: providing a culture comprising embryonic stem cells, contacting said culture with retinoic acid to bias the embryonic stem cells to differentiate along a particular developmental lineage, contacting said culture with one or more test agents, detecting expression of one or more markers which identify the differentiation of an embryonic stem cell to said particular differentiated neuronal cell type. The one or more agents that promote expression of one or more markers of said particular differentiated neuronal cell type are identified as agents that promote the differentiation of an embryonic stem cell to a particular differentiated neuronal cell type. The method further includes the step of synthesizing said compound so identified as an agent that promotes differentiation of an embryonic stem cell to a particular differentiated neuronal cell type.

In one embodiment, the method further comprises formulating said compound in a pharmaceutically acceptable carrier.

In any of the foregoing methods, the invention contemplates that the screening methods can be high-throughput screening methods and/or automated screening methods. Furthermore, the invention contemplates the use of any of the foregoing methods to confirm the physiological relevance (i.e., the ability to promote progressive or terminal differentiation) of agents identified as agonizing or antagonizing a particular signal transduction pathway.

Still furthermore, the invention contemplates that the screening methods can be conducted using modified stem cells (i.e., knockout or transgenic stem cells). In one embodiment, the transgenic stem cells are transgenic embryonic stem cells. In another embodiment, the transgenic embryonic stem cells are transgenic mouse embryonic stem cells. In still another embodiment, the transgenic embryonic stem cells comprise a detectable reporter under the control of a promoter that regulates expression of the reporter upon differentiation of the stem cell to a particular lineage or cell type. In one example, the reporter is regulated by any of a promoter of a gene indicative of neuronal differentiation, a promoter of a gene indicative of mesodermal differentiation, or a promoter of a gene indicative of endodermal differentiation. By way of further example, the reporter is regulated by a motor neuron, interneuron, intermediate neuron, or dopaminergic neuron specific promoter. Exemplary transgenic stem cells express a reporter construct (i.e., a detectable label including GFP, YFP, RFP, alkaline phosphatase, luciferase, etc) regulated by any one of the following: an HB9 promoter, a Math1 promoter, a pdx1 promoter, a nestin promoter, a myf5 promoter, a troponin promoter, a cardiac actin promoter, a HNF3β promoter, a tyrosine hydroxylase promoter, or any other promoter that regulates expression in response to progressive or terminal differentiation along a particular lineage.

In any of the foregoing, the invention contemplates methods which simultaneously or in series can evaluate the ability of a particular agent(s) to promote differentiation to any of several different cell types. Additionally, the invention contemplates the evaluation of both markers of terminal differentiation, as well as markers which identify cells which are more differentiated than a stem cell but not yet terminally differentiated. Such markers lie along the developmental pathway from a progenitor cell to a terminally differentiated cell and can be used in any of a number of ways. The examination of intermediate markers can help identify agents that may themselves be insufficient to terminally differentiate a stem cell or a non-stem cell, but which may be useful in combination with other agents. Similarly, the examination of intermediate markers can help evaluate candidates which may prove sufficient at a different dose.

The practice of the present invention will employ, unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

Other features and advantages of the invention will be apparent from the following detailed description, and from the claims.

### DETAILED DESCRIPTION OF THE DRAWINGS

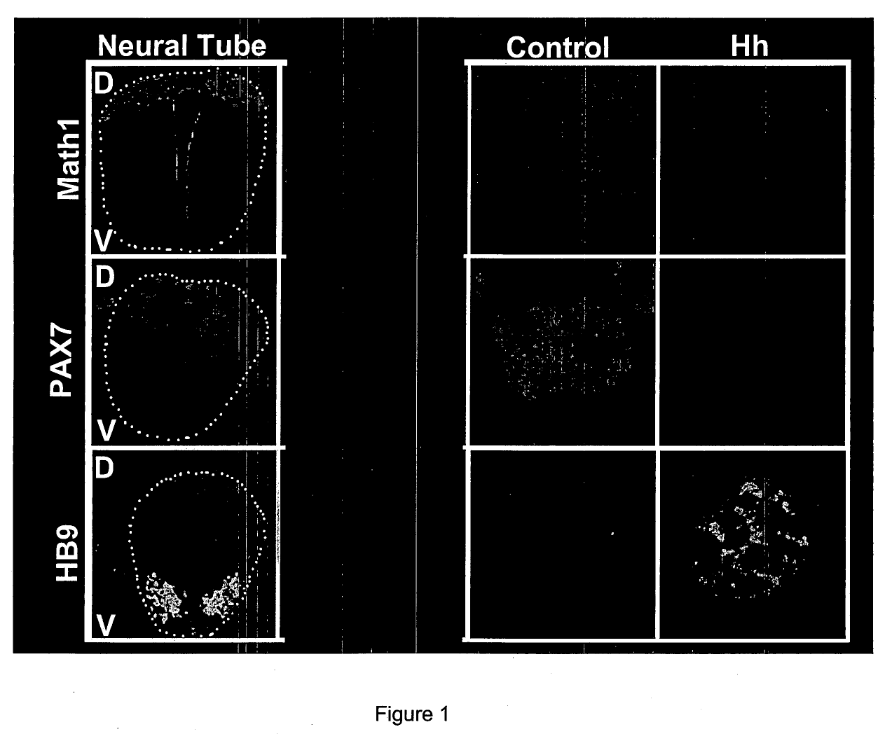

FIG. 1 shows that embryonic stem cells respond to agents and recapitulate differentiation observed in the neural tube. Mouse embryonic stem cells were cultured to confluence, trypsinized, and then allowed to reaggregate to form embryoid bodies. Embryoid bodies were treated with retinoic acid (RA) to promote neuronal differentiation. After culture for one day in the presence of RA, embryoid bodies were either further treated with RA alone, or were cultured in the presence of Sonic hedgehog protein for three days. Treated embryoid bodies were assayed for expression of Math1, a marker of dorsal interneurons; Pax7, a marker of intermediate neurons; or HB9, a marker of motor neurons. Treatment of embryoid bodies with RA alone promoted expression of the intermediate neuronal marker Pax7. Treatment of embryoid bodies with Sonic hedgehog protein promoted expression of the motor neuron marker HB9. For comparison, the left-most panel shows endogenous expression of Math1, Pax7, and HB9 in the neural tube. Dorsal is toward the top of the panel, and is indicated with a “D”. Ventral is toward the bottom of each panel, and is indicated with a “V”.

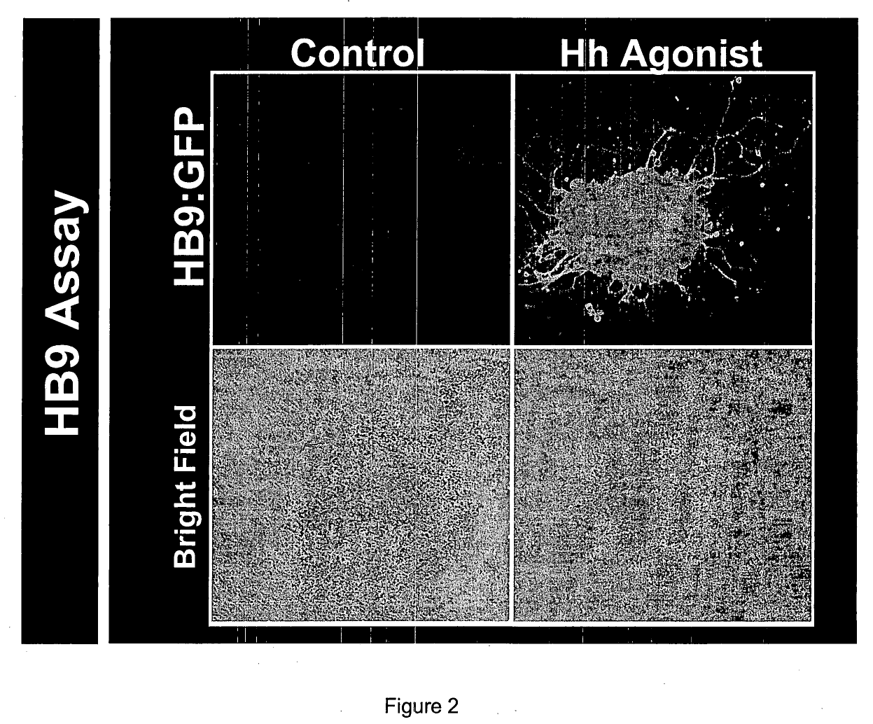

FIG. 2 shows expression of the motor neuron marker HB9 in response to treatment with a hedgehog small molecule agonist in mouse embryonic stem cells expressing a GFP transgene driven by the HB9 promoter. The treated embryonic stem cells not only expressed this marker of motor neuron differentiation, but also extended processes.

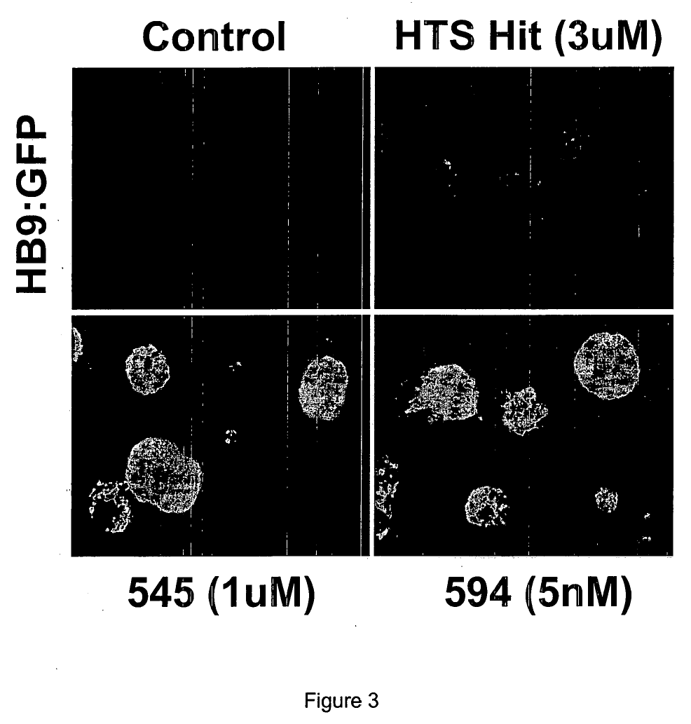

FIG. 3 shows that stem cell based differentiation assays can be used to confirm the biological activity of agents identified using other assays. Briefly, several small molecules were previously identified in a screen to identify agonists of the hedgehog signaling pathway. FIG. 3 shows that three hedgehog agonists (agents that promote hedgehog signal transduction) also promoted differentiation of embryonic stem cells to motor neurons, as assayed by expression of HB9 in (HB9-GFP)-mouse embryonic stem cells.

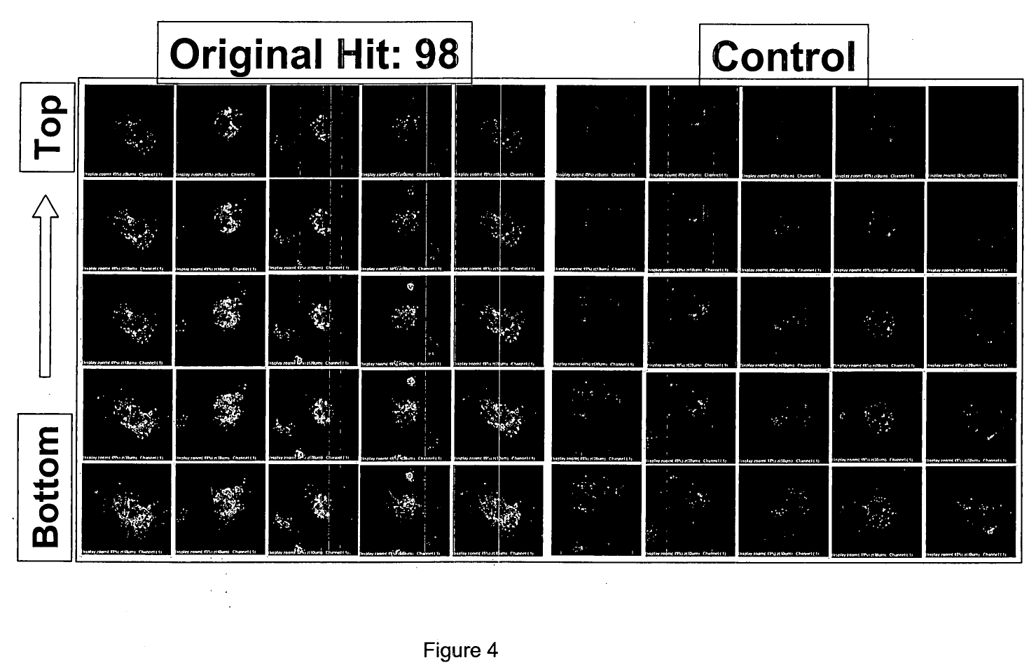

FIG. 4 shows confocal microscopic images of cultures of mouse embryonic stem cells cultured in the presence of a small molecule hedgehog agonist (98) and assayed for expression of HB9. In all sections examined, cultures treated with the hedgehog agonist had more HB9 expressing cells than control cultures.

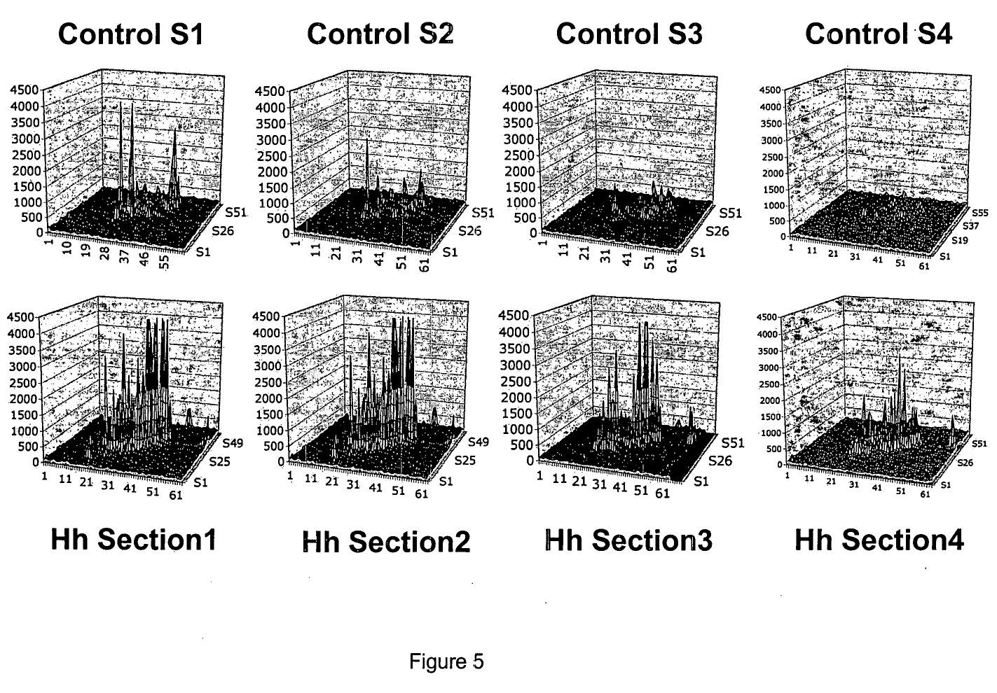

FIG. 5 shows a density profile prepared from the confocal images presented in FIG. 4.

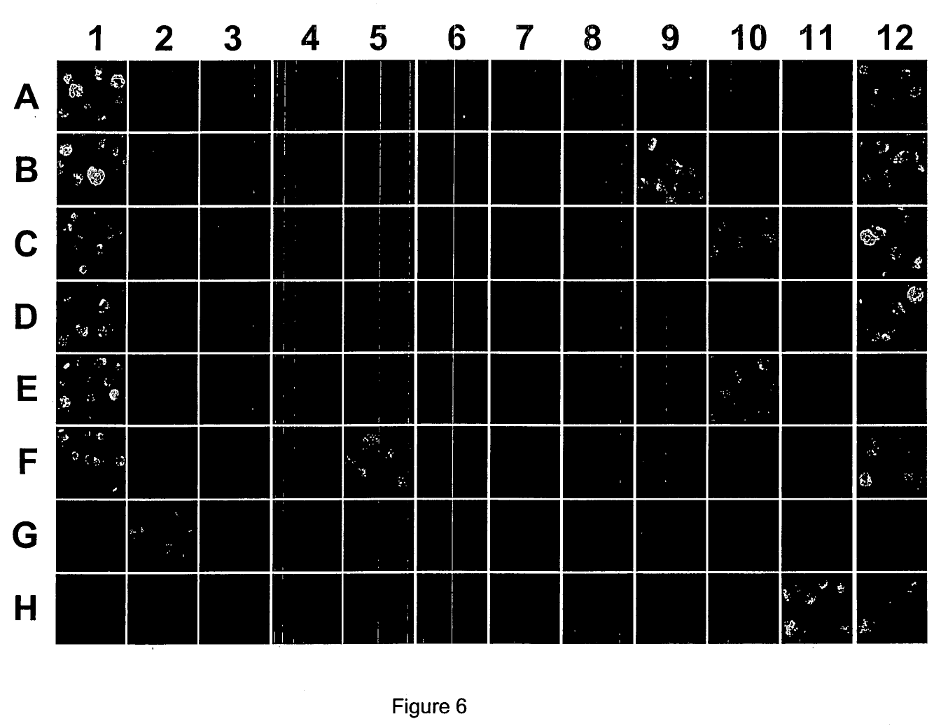

FIG. 6 shows analysis of a mini, small molecule library spiked with seven hedgehog agonists. Mouse embryonic stem cells were used to screen this spiked, mini-library. Embryoid bodies were treated with aliquots of the spiked library, and motor neuron differentiation was assessed by expression of HB9. Expression of HB9 correctly identified the aliquots containing the seven hedgehog agonists (G2, H3, F5, B9, C10, E10, and H11).

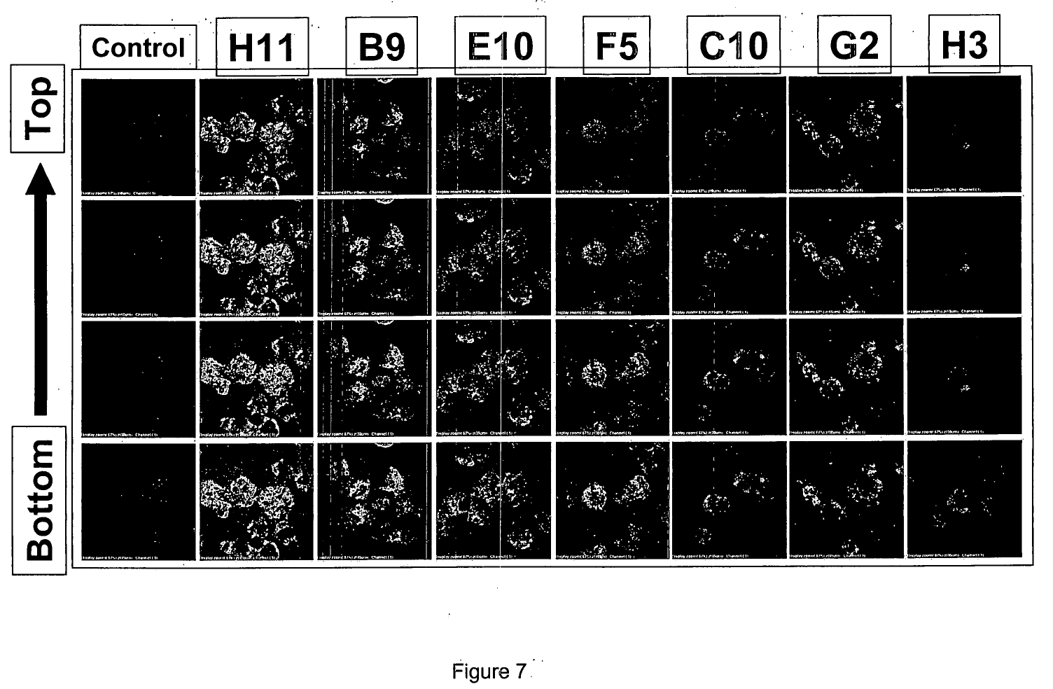

FIG. 7 shows confocal microscopic images of HB9 expression in mouse embryoid bodies cultured with aliquots of the spiked mini-library containing a known hedgehog agonist (G2, H3, F5, B9, C10, E10, and H11). In all sections examined, cultures treated with the hedgehog agonist had more HB9 expressing cells than control cultures.

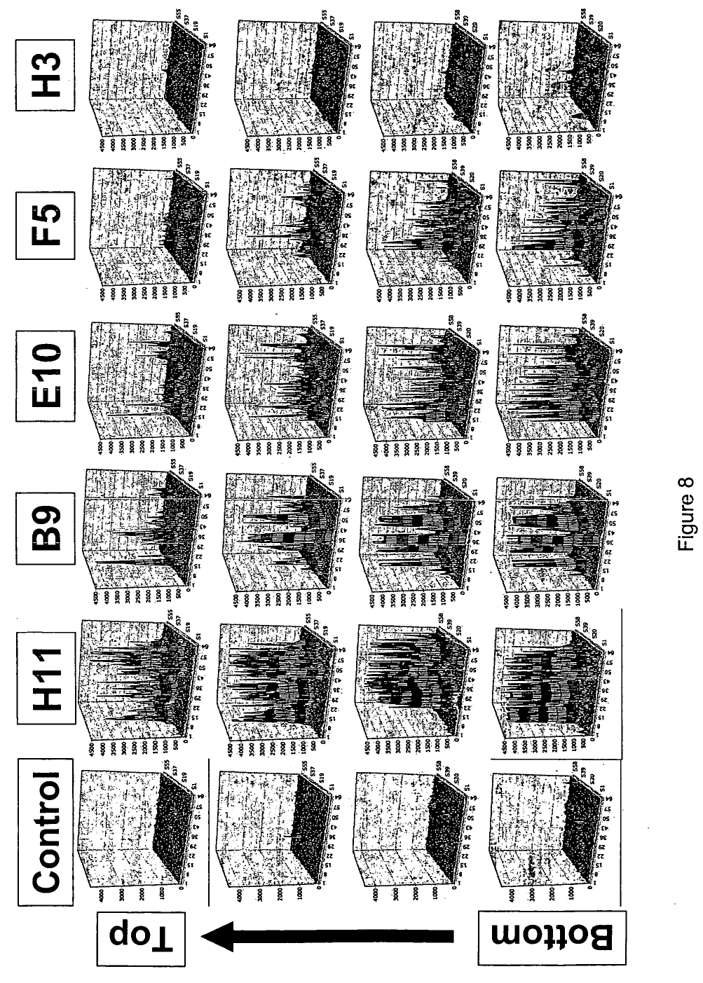

FIG. 8 shows a density profile prepared from the confocal images presented in FIG. 7.

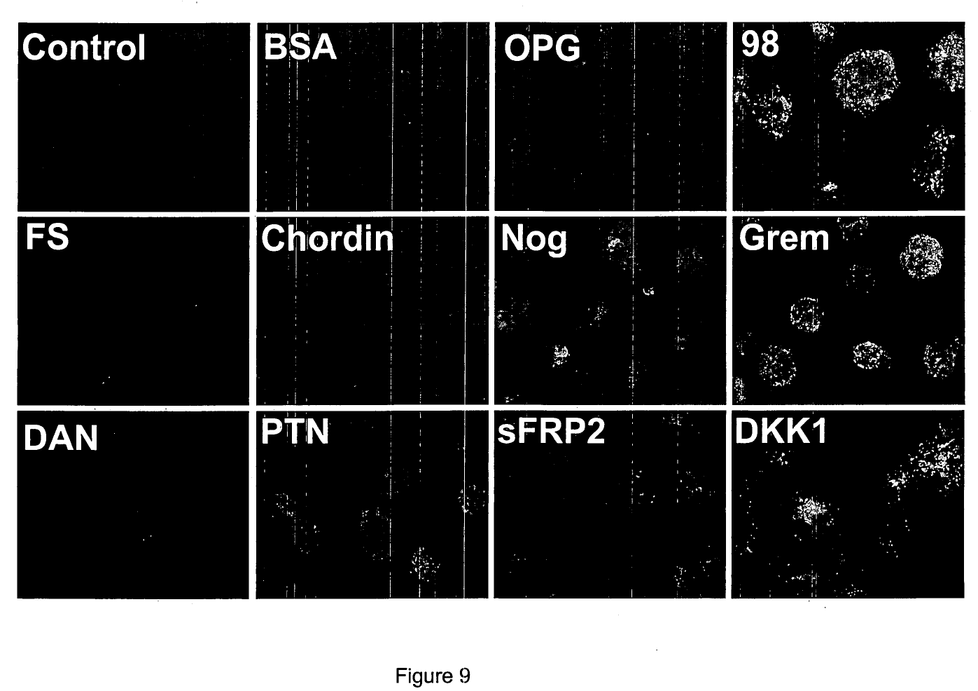

FIG. 9 shows that the methods of the present invention can be used to identify BMP antagonists and Wnt antagonists that promote motor neuron differentiation. Mouse embryonic stem cells expressing GFP under the control of the HB9 promoter were cultured to confluence, trypsinized, and allowed to reaggregate to form embryoid bodies. Subsequently, the embryoid bodies were cultured for three days with either a hedgehog agonist, a BMP antagonist, or a Wnt antagonist.

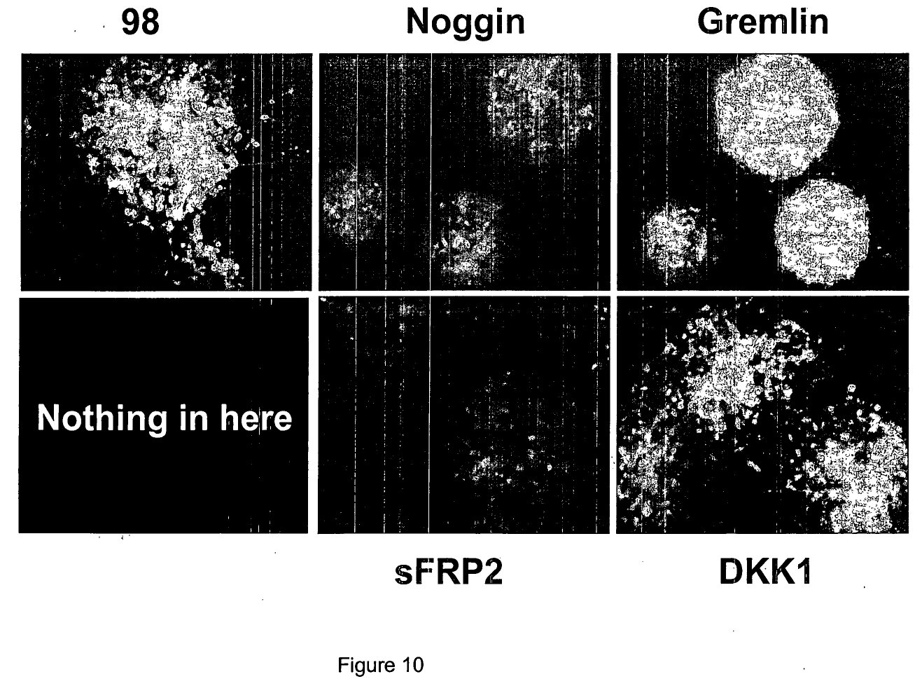

FIG. 10 shows morphological differences among embryoid bodies differentiated using a hedgehog agonist, a BMP antagonist, or a Wnt antagonist.

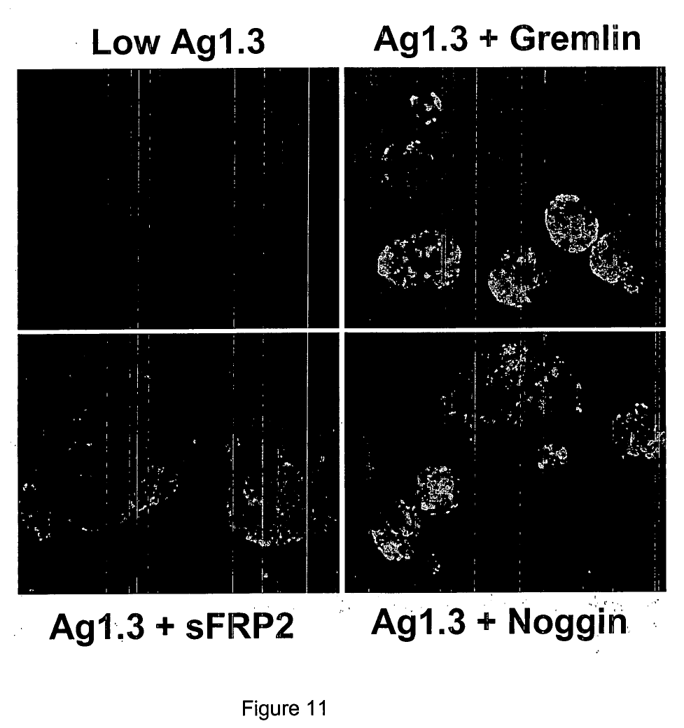

FIG. 11 shows that combinations of agents can synergize to promote differentiation to a particular cell type. BMP antagonists and Wnt antagonists synergized with hedgehog agonists to promote motor neuron differentiation from embryoid bodies. Treatment of embryoid bodies with a sub-threshold level of a small molecule hedgehog agonist did not promote motor neuron differentiation. However, treatment of embryoid bodies with the same sub-threshold concentration of a small molecule hedgehog agonist plus either the Wnt antagonist sFRP2, the BMP antagonist gremlin, or the BMP antagonist noggin promoted motor neuron differentiation.

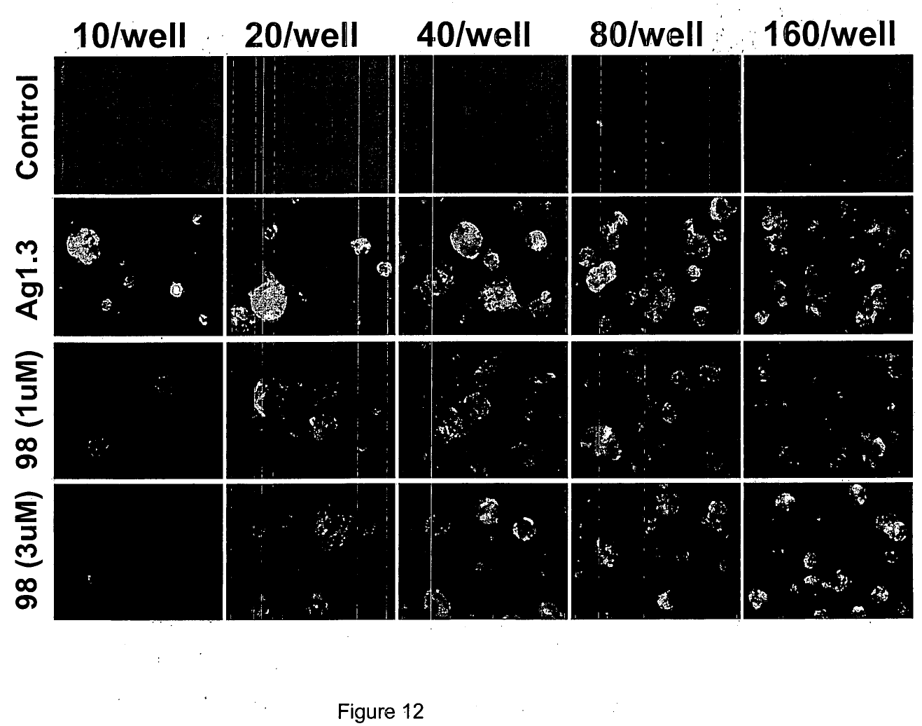

FIG. 12 shows that the stem cell based screening methods of the present invention are amenable to a high-throughput format. The embryonic stem cell screen can be performed in a 384-well format, and at cell densities ranging from 20-160 embryoid bodies per well.

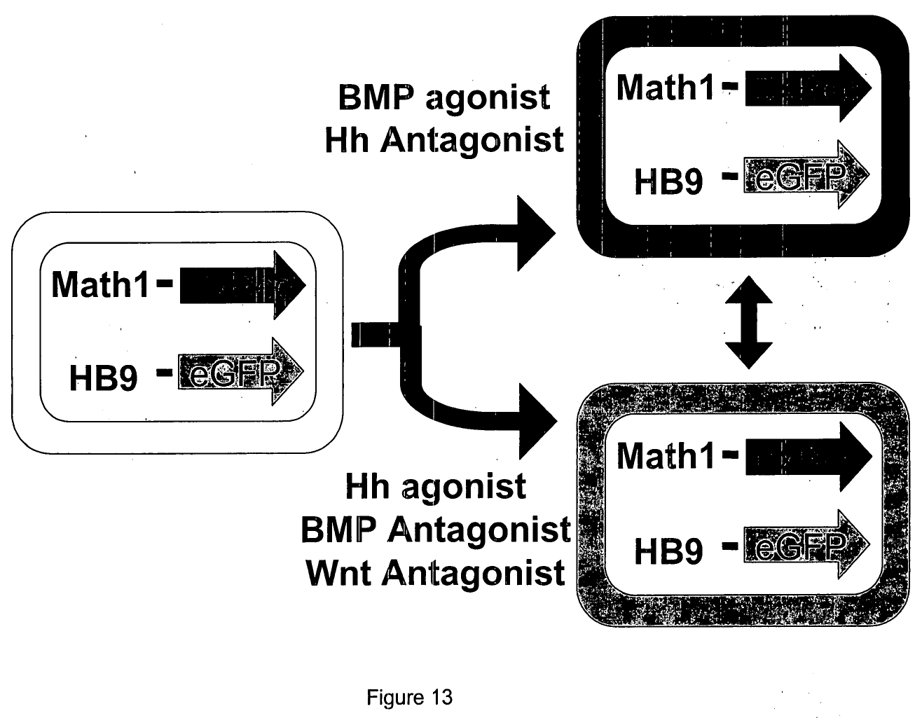

FIG. 13 shows a schematic representation of a multi-plex screening system that could be used in combination with the methods of the present invention to screen agents simultaneously for the ability to promote progressive or terminal differentiation to any one of several cell types.

### DETAILED DESCRIPTION OF THE INVENTION

(i) Overview

The present invention provides novel methods for identifying and characterizing agents that promote the differentiation of cells to particular differentiated cell types. In contrast to previous screening methods known in the art, the methods of the present invention differ in several ways and these differences provide several benefits. Firstly, many currently employed screening assays, regardless of the cell type in which they are performed, require prior knowledge of the mechanism by which a particular agent exerts its function. For example, assays based on the ability of test agents to activate or suppress a particular signaling pathway or assays based on the ability of a test agent to bind to a particular receptor. Such assays can be very robust, however, they require a great deal of mechanistic knowledge of the process by which a sought after agent exerts a desired effect. Secondly, many currently employed assays rely on the detection of a quickly and readily observable process (i.e., binding to a target receptor). However, agents identified in these assays may not correspond to agents which ultimately produce a desired cellular response. Accordingly, many currently employed assays require extensive validation to eliminate “hits” which later are shown to lack the desired cellular/physiological activity.

The present invention provides novel assays unencumbered by many of the limitations which limit the utility of previously employed assay methods. The present invention provides assay methods conducted in stem cells and biased cells (physiologically relevant cell-based systems), and relies upon a function-based read-out (e.g., expression of a marker of terminal differentiation; expression of a marker of progressive differentiation; morphological changes indicative of differentiation; cell cycle changes indicative of differentiation; motility changes indicative of differentiation; changes in adherence indicative of differentiation) to assess whether an agent possesses a desired activity. Furthermore, the assays disclosed herein are amenable to adaptation for high-throughput screening, and are also adaptable to multi-marker (i.e., multi-plex) analysis to assay the ability of a given agent to have any of a number of effects on a particular cell. Furthermore, the assays disclosed herein are amenable to assessment of combinations of read-outs (e.g., analysis of one or more molecular markers plus one or more non-molecular marker readouts) to analyze the ability of an agent to influence the progressive or terminal differentiation of a cell. Finally, the assay methods described herein require no a priori knowledge of the cellular and molecular mechanisms leading from a less committed cell (i.e., a stem cell or a biased cell), to a more committed cell, and finally to a particular differentiated cell type. Nevertheless, the disclosed assay methods are similarly useful for identifying agents that influence cellular differentiation by agonizing or antagonizing a particular signaling pathway, as well as for confirming that an agonist or antagonist of a particular signaling pathway influences differentiation.

(ii) Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wildtype polynucleotide sequence or any change in a wildtype protein sequence. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

“Homology” and “identity” are used synonymously throughout and refer to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

A “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

As used herein, “small organic molecule” refers to compounds smaller than proteins that are generally characterized by the ability to transit cellular membranes more easily than proteins. Preferred small organic molecules are characterized as having a size less than 10,000 AMU. More preferably, between 5000-10,000 AMU. Most preferably, the small organic molecules are characterized as having a size between 1000-5000 AMU.

The “non-human animals” of the invention include mammals such as rats, mice, rabbits, sheep, cats, dogs, cows, pigs, and non-human primates.

The term “isolated” as used herein with respect to nucleic acids, such as DNA or RNA, refers to molecules separated from other DNAs, or RNAs, respectively, that are present in the natural source of the macromolecule. For example, an isolated nucleic acid preferably includes no more than 10 kilobases (kb) of nucleic acid sequence which naturally immediately flanks the gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

As used herein, “proliferating” and “proliferation” refer to cells undergoing mitosis.

As used herein the term “animal” refers to mammals, including mammals such as humans. Likewise, a “patient” or “subject” to be treated by the method of the invention can mean either a human or non-human animal.

“Differentiation” in the present context means the formation of cells expressing markers known to be associated with cells that are more specialized and closer to becoming terminally differentiated cells incapable of further division or differentiation. The pathway along which cells progress from a less committed cell, to a cell that is increasingly committed to a particular cell type, and eventually to a terminally differentiated cell is referred to as progressive differentiation or progressive commitment.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated, or differentiable daughter cells. In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

The term “adult stem cell” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the invention contemplates the use of stem cells isolated from any tissue source.

The term “tissue” refers to a group or layer of similarly specialized cells which together perform certain special functions.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the term “substantially pure” refers to a population of cells that contain fewer than about 20%, more preferably fewer than about 10%, most preferably fewer than about 5%, of lineage committed cells.

The invention further contemplates the screening of libraries of agents. Such libraries may include, without limitation, cDNA libraries (either plasmid based or phage based), expression libraries, combinatorial libraries, chemical libraries, phage display libraries, variegated libraries, and biased libraries. The term “library” refers to a collection of nucleic acids, proteins, peptides, chemical compounds, small organic molecules, or antibodies. Libraries comprising each of these are well known in the art. Exemplary types of libraries include combinatorial, variegated, biased, and unbiased libraries. Libraries can provide a systematic way to screen large numbers of nucleic acids, proteins, peptides, chemical compounds, small organic molecules, or antibodies. Often, libraries are sub-divided into pools containing some fraction of the total species represented in the entire library. These pools can then be screened to identify fractions containing the desired activity. The pools can be further subdivided, and this process can be repeated until either (i) the desired activity can be correlated with a specific species contained within the library, or (ii) the desired activity is lost during further subdivision of the pool of species, and thus is the result of multiple species contained within the library.

As used herein, “neuronal cell” or “cell of the nervous system” include both neurons and glial cells.

As used herein, “CNS neuron” refers to a neuron whose cell body is located in the central nervous system. The term is also meant to encompass neurons whose cell body was originally located in the central nervous system (e.g., endogenously located in the CNS), but which have been explanted and cultured ex vivo, as well as the progeny of such cells. Examples of such neurons are motor neurons, interneurons and sensory neurons including retinal ganglion cells, dorsal root ganglion cells and neurons of the spinal cord.

As used herein, “central nervous system” refers to any of the functional regions of the brain or spinal cord. This definition is used commonly in the art and is based, at least in part, on the common embryonic origin of the structures of the brain and spinal cord from the neural tube.

The “peripheral nervous system” can be distinguished from the central nervous system, at least in part, by its differing origin during embryogenesis. Cells of the peripheral nervous system are derived from the neural crest and include neurons and glia of the sensory, sympathetic and parasympathetic systems.

As used herein, “soma” refers to the cell body of a neuron.

As used herein, “axon” and “neurite” are used interchangeably to refer to the single outgrowth which extends from a neuron and which will ultimately migrate to innervate a target tissue. The tip of the axon is referred to as the “growth cone”. Axons extend from a neuron to a target tissue, and are capable of conducting impulses. In the literature, the term “axon” is often used to refer to the outgrowth from a cell in vivo, and the term “neurite” is often used to refer to the outgrowth from a cell in vitro, however, the terms are used interchangeably herein without regard to whether the cells are found in vivo or in vitro.

As used herein, “dendrite” refers to the fine extensions from a neuron soma which pick up electrical and chemical impulses. The number of dendrites found on a given neuron vary extensively and depend on the specific neuron. Typical neurons may have multiple dendrites, but only a single axon, and it is the axon that migrates in response to cues to innervate a target tissue.

A “marker” is used to determine the state of a cell. Markers are characteristics, whether morphological or biochemical (enzymatic), particular to a cell type, or molecules expressed by the cell type. Preferably, such markers are proteins, and more preferably, possess an epitope for antibodies or other binding molecules available in the art. However, a marker may consist of any molecule found in a cell, including, but not limited to, proteins (peptides and polypeptides), lipids, polysaccharides, nucleic acids and steroids. Additionally, a marker may comprise a morphological or functional characteristic of a cell. Examples of morphological traits include, but are not limited to, shape, size, and nuclear to cytoplasmic ratio. Examples of functional traits include, but are not limited to, the ability to adhere to particular substrates, ability to incorporate or exclude particular dyes, ability to migrate under particular conditions, and the ability to differentiate along particular lineages.

Markers may be detected by any method available to one of skill in the art. In addition to antibodies (and all antibody derivatives) that recognize and bind at least one epitope on a marker molecule, markers may be detected using analytical techniques, such as by protein dot blots, sodium dodecyl sulfate polyacrylamide gel electrophoresis (SDS-PAGE), or any other gel system that separates proteins, with subsequent visualization of the marker (such as Western blots), gel filtration, affinity column purification; morphologically, such as fluorescent-activated cell sorting (FACS), staining with dyes that have a specific reaction with a marker molecule (such as ruthenium red and extracellular matrix molecules), specific morphological characteristics (such as the presence of microvilli in epithelia, or the pseudopodia/filopodia in migrating cells, such as fibroblasts and mesenchyme); and biochemically, such as assaying for an enzymatic product or intermediate, or the overall composition of a cell, such as the ratio of protein to lipid, or lipid to sugar, or even the ratio of two specific lipids to each other, or polysaccharides. In the case of nucleic acid markers, any known method may be used. If such a marker is a nucleic acid, PCR, RT-PCR, in situ hybridization, dot blot hybridization, Northern blots, Southern blots and the like may be used, coupled with suitable detection methods. If such a marker is a morphological and/or functional trait, suitable methods include visual inspection using, for example, the unaided eye, a stereomicroscope, a dissecting microscope, a confocal microscope, or an electron microscope. The invention contemplates methods of analyzing the progressive or terminal differentiation of a cell employing a single marker, as well as any combination of molecular and/or non-molecular markers.

Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (a so called terminally differentiated cell). In many, but not all tissues, the process of differentiation is coupled with exit from the cell cycle. In these cases, the terminally differentiated cells lose or greatly restrict their capacity to proliferate. However, we note that the term “differentiation” or “differentiated” refers to cells that are more specialized in their fate or function than at a previous point in their development, and includes both cells that are terminally differentiated and cells that, although not terminally differentiated, are more specialized than at a previous point in their development. The development of a cell from an uncommitted cell (for example, a stem cell), to a cell with an increasing degree of commitment to a particular differentiated cell type, and finally to a terminally differentiated cell is known as progressive differentiation or progressive commitment.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The phrase “effective amount” as used herein means that the amount of one or more agents which is effective for promoting differentiation of a stem cell to a particular differentiated cell type.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The terms “hedgehog signaling,” “hedgehog signal transduction,” and “hedgehog signaling pathway” are used interchangeably throughout the application to refer to the mechanism whereby hedgehog proteins (Sonic, Desert, Indian hedgehog) influence proliferation, differentiation, migration, and survival of diverse cell types (see, for example, Allendoerfer (2003) Current Opinion Investig Drugs 3: 1742-1744; Ingham (2001) Genes & Dev 15: 3059-3087). Agents that promote hedgehog signal transduction are referred to as “hedgehog agonists” or “agonists of hedgehog signaling.” Agents that inhibit hedgehog signal transduction are referred to as “hedgehog antagonists” or “antagonists of hedgehog signaling.” Although during normal development hedgehog signal transduction may be influenced by hedgehog protein, the invention contemplates that exemplary agents for use and identified in the present methods include agents that agonize or antagonize hedgehog signal transduction at any point in the pathway (extracellularly, at the cell surface, or intracellularly). For further examples see U.S. Pat. No. 6,444,793; U.S. Pat. No. 6,683,108; U.S. Pat. No. 6,683,198; U.S. Pat. No. 6,686,388; WO02/30421; WO02/30462; WO03/011219; WO03/027234; WO04/020599. Each of the foregoing references are hereby incorporated by reference in their entirety.

The terms “BMP signaling,” “BMP signal transduction,” and “BMP signaling pathway” are used interchangeably throughout the application to refer to the mechanism whereby BMP proteins influence proliferation, differentiation, migration, and survival of diverse cell types (see, for example, Balemans (2002) Developmental Biology 250: 231-250; U.S. Pat. No. 6,498,142; Miyazawa et al. (2002) Genes Cell 7: 1191-1204). Agents that promote BMP signal transduction are referred to as “BMP agonists” or “agonists of BMP signaling.” Agents that inhibit BMP signal transduction are referred to as “BMP antagonists” or “antagonists of BMP signaling.” Although during normal development BMP signal transduction may be influenced by BMP protein, the invention contemplates that exemplary agents for use and identified in the present methods include agents that agonize or antagonize BMP signal transduction at any point in the pathway (extracellularly, at the cell surface, or intracellularly).

The terms “Wnt signaling,” “Wnt signal transduction,” and “Wnt signaling pathway” are used interchangeably throughout the application to refer to the mechanism whereby Wnt proteins influence proliferation, differentiation, migration, and survival of diverse cell types (see, for example, WO02/44378; Wharton (2003) Developmental Biology 253: 1-17). Agents that promote Wnt signal transduction are referred to as “Wnt agonists” or “agonists of Wnt signaling.” Agents that inhibit Wnt signal transduction are referred to as “Wnt antagonists” or “antagonists of Wnt signaling.” Although during normal development Wnt signal transduction may be influenced by a Wnt protein, the invention contemplates that exemplary agents for use and identified in the present methods include agents that agonize or antagonize Wnt signal transduction at any point in the pathway (extracellularly, at the cell surface, or intracellularly).

The terms “Notch signaling,” “Notch signal transduction,” and “Notch signaling pathway” are used interchangeably throughout the application to refer to the mechanism whereby Notch proteins influence proliferation, differentiation, migration, and survival of diverse cell types (see, for example, Baron (2003) Sem Cell Dev Bio 14: 113-119). Agents that promote Notch signal transduction are referred to as “Notch agonists” or “agonists of Notch signaling.” Agents that inhibit Notch signal transduction are referred to as “Notch antagonists” or “antagonists of Notch signaling.” Although during normal development Notch signal transduction may be influenced by a Notch protein, the invention contemplates that exemplary agents for use and identified in the present methods include agents that agonize or antagonize Notch signal transduction at any point in the pathway (extracellularly, at the cell surface, or intracellularly).

(iii) Screening Assays

This application describes methods for identifying and/or characterizing agents that promote the differentiation of a stem cell to a particular differentiated cell type. This application further describes methods for identifying and/or characterizing agents that promote the differentiation of a non-stem cell (i.e., a biased cell or a committed cell) to a particular differentiated cell type. This application further describes methods for identifying and/or characterizing agents that promote the progressive, step-wise differentiation of a cell (either a stem cell or a non-stem cell) to a cell of increasingly greater commitment to a particular differentiated cell type, and finally to a terminally differentiated cell type. Exemplary agents (e.g., a single agent, a combination of two or more agents, a library of agents) include nucleic acids, peptides, polypeptides, peptidomimmetics, antibodies, antisense RNAs, RNAi constructs (including siRNAs), ribozymes, chemical compounds, and small organic molecules. Agents may be screened individually, in combination, or as a library of agents. Without being bound by theory, the invention contemplates that the differentiation of a stem cell to a particular differentiated cell type may involve the activation of particular genes and signaling pathways which promote differentiation along a particular lineage, or the inhibition of particular genes and signaling pathways which function to prevent differentiation along a particular lineage. Accordingly, the present invention contemplates screening a variety of agents such that agents can be identified based on their function (i.e., ability to promote differentiation to a particular cell type) and not based on their mechanism of action. Additionally, however, the screening methods of the present invention can be used to identify agents that promote differentiation to a particular cell type by agonizing or antagonizing a particular signaling pathway. Such methods are useful for identifying agonists or antagonists of a particular signaling pathway (e.g., hedgehog agonists, hedgehog antagonists, Wnt agonists, Wnt antagonists, BMP agonists, BMP antagonists, Notch agonist, Notch antagonists).

In many drug screening programs that test libraries of nucleic acids, polypeptides, chemical compounds and natural extracts, high throughput assays are desirable to increase the number of agents surveyed in a given period of time. To this end, many screens rely upon either cell-free systems or cell-based systems designed to maximize convenience with respect to the particular cell type used or the read-out used to evaluate compounds. Although such assays may be useful, and may identify particular agents which meet the criteria of the assay, such assays have limitations.

Cell free assays, or screens conducted in cells which are substantially different than the cells in which the identified agents will ultimately be used, may identify as “hits” compounds which will not have the desired activity when used in the proper cellular context. Accordingly, although such assays may often provide a convenient primary screen, the results obtained must always be verified in one or more additional models.

The read-out in many cell-free and cell-based screens is purposefully chosen to maximize convenience or to evaluate agents possessing a very particular mechanism of action. Accordingly, screens conducted in this way either require knowledge of the mechanism via which the desired compounds function, or the willingness to base the screen on particular assumptions which will bias the types of compounds ultimately identified.

The present invention provides cell based screening methods to identify and characterize agents that promote the differentiation of a cell to a particular differentiated cell type. Exemplary screens may be performed using embryonic stem cells, any of a number of adult stem cells, or non-stem cells. Suitable adult stem cells are well known in the art and include neural stem cells, neural crest stem cells, mesenchymal stem cells, hematopoietic stem cells, hepatic stem cells, cardiac stem cells, epidermal stem cells, and pancreatic stem cells. Furthermore, stem cells are thought to reside in virtually all adult tissues, and thus suitable adult stem cells also include stem cells isolated from adult tissues including, but not limited to, hair follicle, skin, tongue, skeletal muscle, kidney, small intestine, large intestine, esophagus, lung, bone marrow, blood, ovaries, breast, testes, and uterus. By conducting the assay in any of these physiologically relevant cell populations, agents identified using this assay are more likely to behave similarly in other physiological contexts—such as in vivo.

The present invention further provides cell based screening methods designed to evaluate the ability of agents to promote differentiation (i.e., terminal differentiation or simply a more committed/differentiated state) of a cell to a particular differentiated cell type. The read-out of this assay is the expression of one or more markers (e.g., molecular markers and or non-molecular markers) of a particular differentiated cell type. Thus, the assay is specific for agents that have a specific cellular and physiological effect on cells. Additionally, the assay is unbiased with respect to the types of agents identified or the mechanism by which an agent exerts its effects on the cells. Additionally, the methods of the present invention can also be used, alone or in combination with other assays, to identify agents that promote progressive and/or terminal differentiation to a particular cell type by agonizing or antagonizing one or more signaling pathways. Accordingly, the present invention can be used to identify differentiation agents, as well as to identify differentiation agents that agonize or antagonize particular signaling pathways. By way of example, the present methods can be used to identify and/or confirm that an agent is a hedgehog agonist, a hedgehog antagonist, a Wnt agonist, a Wnt antagonist, a BMP agonist, a BMP antagonist, a Notch agonist, a Notch antagonist, and the like.

Agents can be screened individually, in combination with one or more other agents, or as a library of agents. Agents include nucleic acids, peptides, polypeptides, peptidomimmetics, RNAi constructs, antisense oligonucleotides, ribozymes, antibodies, and small organic molecules.

To illustrate an exemplary screening assay, a culture of embryonic stem cells is provided. Cells are aggregated to form embryoid bodies, however, in certain embodiments the cells need not be aggregated prior to the screening steps. Embryoid bodies are contacted with a composition comprising a biasing factor. Such a biasing factor helps initially tip the embryonic stem cell down a particular developmental lineage (i.e., ectodermal, mesodermal, endodermal). In one embodiment, the biasing factor is retinoic acid, and contacting the embryoid bodies with retinoic acid biases the cells generally along an ectodermal lineage and specifically along a neuronal lineage. The biased embryoid bodies are contacted with one or more agents (for example, a library of agents). The embryoid bodies can be contacted with the one or more agents simultaneously with the retinoic acid or soon after treatment with retinoic acid. Following treatment with the test agents, the embryoid bodies are examined using markers of particular differentiated neuronal cell types.

In one embodiment, embryoid bodies are examined over several days to assess progression of cells within the embryoid bodies to more committed neuronal cells and finally to terminally differentiated cells. In another embodiment, the cells are assayed at a particular time point following treatment using markers of several different differentiated cell type (e.g., so called multi-plex evaluation). In this way, the ability of a single factor or pool of factors to influence differentiation along any of a number of lineages can be simultaneously evaluated.

The invention contemplates any of a number of methods for detecting expression of a marker of differentiation. When the marker of differentiation is a protein, expression can be measured by immunocytochemistry using an antibody immunoreactive with the particular protein. Similarly, such antibodies can be used to perform Western blot analysis. When the marker is a nucleic acid, expression can be measured by RT-PCR, Northern blot analysis, RNAse protection, or in situ hybridization. “Positives” can be scored via visual inspection or in an automated matter by FACS analysis or other form of optical scanning. In addition, the invention contemplates the use of transgenic stem cells expressing a reporter construct which can be used as a marker. For example, cells containing a reporter construct such that a detectable marker is expressed in cells which differentiate along a particular lineage (see, for example, Wichterle et al.), or cells that contain multiple such reporter constructs such that the ability of a test agent to promote differentiation along any of a number of lineages can be simultaneously evaluated. Furthermore, the markers contemplated for use in the methods of the present invention include non-molecular markers. Such non-molecular markers can be used alone or in combination with one or more molecular markers. Exemplary non-molecular markers include, without limitation, morphology (e.g., size, shape) cell cycle status, migration, adherence, etc.

By way of another example, agents can be screened using neural stem cells. Methods of culturing neural stem cells, either as non-adherent clusters known as neurospheres or as adherent cultures, are well known in the art. A culture comprising neural stem cells is provided. The following steps can be performed on either aggregated clusters of neural stem cells (neurospheres) or on adherent cultures of neural stem cells. The neural stem cells can already be thought to be “biased” along a neuronal lineage. However, these cells can be further primed to terminally differentiate along a neuronal lineage by contacting the cells with a neuronal biasing factor such as a composition comprising retinoic acid. The cells are then (either at the same time or following) contacted with one or more test agents, and the ability of the agents to promote differentiation to a particular neuronal cell type is assessed as described.

The efficacy of the agent can be assessed by generating dose response curves from data obtained using various concentrations of the test agent. Moreover, a control assay can also be performed to provide a baseline for comparison. Such candidates can be further tested for efficacy in promoting differentiation in other in vitro systems, as well as in in vivo models.

The above described methods are amenable to high-throughput analysis, for example, the screen can be conducted in multi-well plates (96-well or greater). Additionally, the use of either transgenic cells containing one or more reporter constructs, or the use of antibody-based detection using a FACS-sortable detectable label facilitates rapid and automated evaluation.

In addition to the stem cell based assays described above, agents may be evaluated in vivo using wildtype animals or animal models of the particular diseases potentially treatable by agents which promote differentiation of stem cells to particular cell types. Animal models may be used as a primary screen or animal models may be used as a secondary screen to evaluate the possible application of the identified agents in a whole animal (possibly therapeutic) context.

For example, agents identified as capable of promoting differentiation of a stem cell to a neuronal cell type (i.e., dopaminergic neuron, motor neuron, sensory neuron, Schwann cell, astrocyte, oligodendrocyte, and the like) can be evaluated in a wildtype mouse, or in a mouse model of a neurodegenerative disease or injury. The candidate agent is administered to a mouse, wherein the mouse is a mouse model of a neurodegenerative disease. Following administration of the agent, the mouse is examined to assess neurological function (in comparison to function prior to administration of the agent and in comparison to a mouse administrated a placebo). The mouse is further examined post mortem to assess changes in neuronal patterning, changes at the cite of injury, changes in cell proliferation, changes in cell survival, etc. Without being bound by theory, agents that promote differentiation of stem cells in culture may promote differentiation of endogenous stem cells when administered to an animal. However, it is also possible that such agents will promote differentiation in vivo by other means such as by promoting differentiation of committed cells that are not yet terminally differentiated, or by promoting the survival of particular cell populations.

Further exemplary cell based screening assays are detailed in the examples. In any of these cell based assays, the invention contemplates the screening of any of a number of nucleic acid, polypeptide, and small organic molecule based agents. The invention further contemplates the identification of agents sufficient to terminally differentiate a stem or non-stem cell, as well as agents sufficient to promote the progressive differentiation of a stem or non-stem cell along a particular developmental lineage. The methods of the present invention are particularly useful for identifying agents that modulate the progressive or terminal differentiation of cell to a particular fate without the need for any knowledge of the mechanisms required for differentiation along that lineage. Additionally, however, the methods of the present invention are suitable for identifying or confirming that an agent that influences progressive or terminal differentiation of a cell does so by agonizing or antagonizing a particular signal transduction pathway. By way of example, the methods of the present invention are useful for identifying or confirming the activity of agonists of the hedgehog signaling pathway, antagonists of the hedgehog signaling pathway, agonists of the Wnt signaling pathway, antagonists of the Wnt signaling pathway, agonists of the BMP signaling pathway, antagonists of the BMP signaling pathway, agonists of the Notch signaling pathway, antagonists of the Notch signaling pathway. The invention contemplates methods of identifying agents that influence cell fate by modulating (agonizing or antagonizing) signal transduction via a particular signaling pathway, as well as the use of such agents in vitro or in vivo to agonize or antagonize that signal transduction pathway, and thereby influence cell fate.

(iv) Exemplary Compositions

The present invention contemplates screening to identify and/or characterize agents that promote differentiation of a cell to a particular differentiated cell type. The methods provided herein are designed to screen any of a variety of agents without regard to the mechanism of action of those agents. For example, the invention contemplates that agents that promote differentiation of a cell to a differentiated cell type may work by promoting expression of a particular gene or protein or by activating signal transduction through a particular signaling pathway. Similarly, agents that promote differentiation of a cell to a differentiated cell type may work by inhibiting expression of a gene or protein or by inhibiting signaling through a signal transduction pathway that normally functions to antagonize differentiation of the cell to a particular differentiated cell type.

The methods of the present invention are particularly useful for identifying agents that promote differentiation without regard to the mechanism of action of the agent. However, the present invention is similarly useful for identifying agents that promote differentiation via particular signaling pathways known to mediate particular steps in the differentiation process. By way of example, the BMP and Wnt signaling pathways are known to function in promoting dorsalization of the developing neural tube. Similarly, these signaling pathways promote differentiation to a dorsal, neural cell fate in embryonic stem cells. Thus, agents that are putative agonists of the BMP or Wnt signaling pathways can be identified by screening to identify agents that promote differentiation of a dorsal, neural cell fate. Conversely, inhibition of BMP signaling or Wnt signaling inhibits the dorsal, neural cell fate while promoting differentiation to a ventral, neural cell fate. Thus, agents that are putative antagonists of the BMP or Wnt signaling pathways can be identified by screening to identify agents that promote differentiation of a ventral neural cell fate.

Agents screened by the methods of the present invention include nucleic acids, peptides, polypeptides, small organic molecules, antibodies, antisense oligonucleotides, RNAi constructs, and ribozymes. These classes of agents are described more thoroughly throughout the application. However, we note that because these methods are based on analysis of the differentiation state of the stem cell in response to the agents, and not on the mechanism by which the agent functions, the screens described herein require no a priori knowledge of the agents which are sufficient to promote differentiation along a particular lineage.

A. Classes of Agents

Numerous mechanisms exist to promote or inhibit the expression and/or activity of a particular mRNA or protein. The following are illustrative examples of exemplary classes of agents that promote or inhibit expression and/or activity of nucleic acids or proteins or that promote or inhibit signal transduction via a signaling pathway. These examples are in no way meant to be limiting, and one of skill in the art can readily select from among known methods for promoting or inhibiting expression and/or activity.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An -anomeric oligonucleotide forms specific double-stranded hybrids with complementary RNA in which, contrary to the usual -units, the strands run parallel to each other (Gautier et al., 1987, Nucl. Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related protein such as isoforms or functionally redundant protein. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:3942), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Ribozyme molecules designed to catalytically cleave an mRNA transcript can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes unlike antisense molecules, are catalytic, a lower intracellular concentration is required for efficiency.

Antibodies can be used as inhibitors of the activity of a particular protein. Antibodies can have extraordinary affinity and specificity for particular epitopes. Antibodies that bind to a particular protein in such a way that the binding of the antibody to the epitope on the protein can interfere with the function of that protein. For example, an antibody may inhibit the function of the protein by sterically hindering the proper protein-protein interactions or occupying active sites. Alternatively the binding of the antibody to an epitope on the particular protein may alter the conformation of that protein such that it is no longer able to properly function.

Monoclonal or polyclonal antibodies can be made using standard protocols (See, for example, Antibodies: A Laboratory Manual ed. by Harlow and Lane (Cold Spring Harbor Press: 1988)). A mammal, such as a mouse, a hamster, a rat, a goat, or a rabbit can be immunized with an immunogenic form of the peptide. Techniques for conferring immunogenicity on a protein or peptide include conjugation to carriers or other techniques well known in the art.

Following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a particular polypeptide and monoclonal antibodies isolated from a culture comprising such hybridoma cells.

The term antibody as used herein is intended to include fragments thereof which are also specifically reactive with a particular polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific and chimeric molecules having affinity for a particular protein conferred by at least one CDR region of the antibody.

Both monoclonal and polyclonal antibodies (Ab) directed against a particular polypeptides, and antibody fragments such as Fab, F(ab)₂, Fv and scFv can be used to block the action of a particular protein. Such antibodies can be used either in an experimental context to further understand the role of a particular protein in a biological process, or in a therapeutic context.

In addition to the use of antibodies as agents, the present invention contemplate that antibodies raised against a particular protein can also be used to monitor the expression of that protein in vitro or in vivo (e.g., such antibodies can be used in immunohistochemical staining). In any of the foregoing, the invention contemplates that antibodies can be readily humanized to make them suitable for administration to human patients.

Peptides, polypeptides, variants polypeptides, and peptide fragments can be agents. Exemplary polypeptides comprise an amino acid sequence at least 60%, 70%, 75%, 80%, 85%, 90%, 95%, 98%, 99%, or 100% identical to a particular polypeptide. Exemplary fragments include fragments of at least 5, 6, 7, 8, 9, 10, 15, 20, 25, 50, 75, 100, 125, 150, 200, 250, or greater than 250 amino acid residues of the full length polypeptide. We note that peptide and polypeptide agents can promote differentiation to a particular differentiated cell type by acting as either an agonist or an antagonist.

Small organic molecules can either agonize or antagonize the function of a particular protein. By small organic molecule is meant a carbon contain molecule having a molecular weight less than 2500 amu, more preferably less than 1500 amu, and even more preferably less than 750 amu. In the context of the present invention, such small organic molecules would be able to promote the differentiation of a cell to a particular differentiated cell type. Without being bound by theory, the small organic molecule may influence cell differentiation by either agonizing the expression and/or activity of a protein or signaling pathway, or by antagonizing the expression and/or activity of a protein or signaling pathway.

Small organic molecules can be readily identified by screening libraries of organic molecules and/or chemical compounds to identify those compounds that have a desired function. Without being bound by theory, small organic molecules may exert their inhibitory function in any of a number of ways. By way of example, small organic molecules may act at the cell surface to influence cell surface receptors. By way of further example, small organic molecules may act intracellularly to influence intracellular signaling along a particular signaling pathway. The methods of the present invention are unbiased and allow identification of small molecule agents that modulate the progressive or terminal differentiation of a cell regardless of the signaling pathways that modulate the particular cell fate. Furthermore, the methods of the present invention are unbiased and allow identification of small molecule agents that act extracellularly, at the cell surface, or intracellularly to modulate cell fate.

In addition to agents which are peptides or polypeptides, the invention contemplates nucleic acids comprising nucleotide sequences encoding peptides and polypeptides. The term nucleic acid as used herein is intended to include equivalents. The term equivalent is understood to include nucleotide sequences which are functionally equivalent to a particular nucleotide sequence. Equivalent nucleotide sequences will include sequences that differ by one or more nucleotide substitutions, additions or deletions, such as allelic variants, and variation due to degeneracy of the genetic code. Equivalent sequences may also include nucleotide sequences that hybridize under stringent conditions (i.e., equivalent to about 20-27° C. below the melting temperature (Tₘ) of the DNA duplex formed in about 1M salt) to a given nucleotide sequence. Further examples of stringent hybridization conditions include a wash step of 0.2×SSC at 65° C.

Nucleic acids having a sequence that differs from nucleotide sequences which encode a particular antagonistic peptide or polypeptide due to degeneracy in the genetic code are also within the scope of the invention. Such nucleic acids encode functionally equivalent peptides but differ in sequence from wildtype sequences known in the art due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist. We note that nucleic acid agents can promote differentiation to a particular differentiated cell type by acting as either an agonist or an antagonist.

(v) Exemplary Methods

The systems and methods described herein also provide vectors containing a nucleic acid, operably linked to at least one transcriptional regulatory sequence. Such vectors may be used, for example, for expressing a polypeptide agent in a cell or for making a probe for the detection of a marker of differentiation. The invention contemplates that certain vectors may be suitable for any of a number of purposes while other vectors are most appropriate for only certain embodiments of the invention. One of skill in the art can readily select from amongst available vectors, as well as select whether the vector should include all or only a portion of a nucleic acid sequence corresponding to a particular gene.

Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences may be used in these vectors to express nucleic acid sequences encoding the agents of this invention. Such useful expression control sequences, include, for example, a viral LTR, such as the LTR of the Moloney murine leukemia virus, the LTR of the Herpes Simplex virus-1, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs can be used to deliver nucleic acids encoding the subject polypeptides. Thus, another aspect of the invention features expression vectors for in vivo or in vitro transfection, viral infection and expression of a subject polypeptide in particular cell types.

This application also describes methods for producing the subject polypeptides. For example, a host cell transfected with a nucleic acid vector directing expression of a nucleotide sequence encoding the subject polypeptides can be cultured under appropriate conditions to allow expression of the peptide to occur. The polypeptide may be secreted and isolated from a mixture of cells and medium containing the recombinant polypeptide. Alternatively, the peptide may be expressed cytoplasmically and the cells harvested, lysed and the protein isolated. A cell culture includes host cells, media and other by-products. Suitable media for cell culture are well known in the art. The recombinant polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In one example, the recombinant polypeptide is a fusion protein containing a domain which facilitates its purification, such as a GST fusion protein. In another example, the subject recombinant polypeptide may include one or more additional domains which facilitate immunodetection, purification, and the like. Exemplary domains include HA, FLAG, GST, His, and the like. Further exemplary domains include a protein transduction domain (PTD) which facilitates the uptake of proteins by cells.

This application also describes a host cell which expresses a recombinant form of the subject polypeptides. The host cell may be a prokaryotic or eukaryotic cell. Thus, a nucleotide sequence derived from the cloning of a protein encoding all or a selected portion (either an antagonistic portion or a bioactive fragment) of the full-length protein, can be used to produce a recombinant form of a polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known proteins, e.g. insulin, interferons, human growth hormone, IL-1, IL-2, and the like. Similar procedures, or modifications thereof, can be employed to prepare recombinant polypeptides by microbial means or tissue-culture technology in accord with the subject invention. Such methods are used to produce experimentally useful proteins that include all or a portion of the subject nucleic acids. For example, such methods are used to produce fusion proteins including domains which facilitate purification or immunodetection, and to produce recombinant forms of a protein.

The recombinant genes can be produced by ligating a nucleic acid encoding a protein, or a portion thereof, into a vector suitable for expression in either prokaryotic cells, eukaryotic cells, or both. Expression vectors for production of recombinant forms of the subject polypeptides include plasmids and other vectors. For instance, suitable vectors for the expression of a polypeptide include plasmids of the types: pBR322-derived plasmids, pEMBL-derived plasmids, pEX-derived plasmids, pGEX-derived plasmids, pTrc-His-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae.

Many mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo, pBacMam-2, and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. For other suitable expression systems for both prokaryotic and eukaryotic cells, as well as general recombinant procedures, see Molecular Cloning A Laboratory Manual, 3rd Ed., ed. by Sambrook and Russell (Cold Spring Harbor Laboratory Press: 2001).

In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

When it is desirable to express only a portion of a protein, such as a form lacking a portion of the N-terminus, e.g. a truncation mutant, it may be necessary to add a start codon (ATG) to the oligonucleotide fragment containing the desired sequence to be expressed. It is well known in the art that a methionine at the N-terminal position can be enzymatically cleaved by the enzyme methionine aminopeptidase (MAP).

Techniques for making fusion genes are known to those skilled in the art. The joining of various nucleic acid fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another example, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence.

The present invention also makes available isolated polypeptides which are isolated from, or otherwise substantially free of other cellular and extracellular proteins. The term “substantially free of other cellular or extracellular proteins” (also referred to herein as “contaminating proteins”) or “substantially pure or purified preparations” are defined as encompassing preparations having less than 20% (by dry weight) contaminating protein, and preferably having less than 5% contaminating protein. Functional forms of the subject polypeptides can be prepared as purified preparations by using a cloned gene as described herein. By “purified”, it is meant, when referring to peptide or nucleic acid sequences, that the indicated molecule is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water and buffers can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

Isolated peptidyl portions of proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. The recombinant polypeptides of the present invention also include versions of those proteins that are resistant to proteolytic cleavage. Variants of the present invention also include proteins which have been post-translationally modified in a manner different than the authentic protein. Modification of the structure of the subject polypeptides can be for such purposes as enhancing therapeutic or prophylactic efficacy, or stability (e.g., ex vivo shelf life and resistance to proteolytic degradation in vivo).

For example, it is reasonable to expect that, in some instances, an isolated replacement of a leucine with an isoleucine or valine, an aspartate with a glutamate, a threonine with a serine, or a similar replacement of an amino acid with a structurally related amino acid (e.g., isosteric and/or isoelectric mutations) may not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 5th ed. by Berg, Tymoczko and Stryer, WH Freeman and Co.: 2002). Whether a change in the amino acid sequence of a peptide results in a variant which maintains the same function as the wildtype protein, or a variant which antagonizes the function of the wildtype protein, can be determined by assessing the ability of the variant peptide to produce a response in cells in a fashion similar to the wild-type protein, or antagonize such a response. Polypeptides in which more than one replacement has taken place can readily be tested in the same manner.

Advances in the fields of combinatorial chemistry and combinatorial mutagenesis have facilitated the making of polypeptide variants (Wissmanm et al. (1991) Genetics 128: 225-232; Graham et al. (1993) Biochemistry 32: 6250-6258; York et al. (1991) Journal of Biological Chemistry 266: 8495-8500; Reidhaar-Olson et al. (1988) Science 241: 53-57). Given one or more assays for testing polypeptide variants, one can assess whether a given variant functions as an antagonist, or whether a given variant has the same or substantially the same function as the wildtype protein. In the context of the present invention, several methods for assaying the functional activity of potential variants are provided.

To further illustrate, the invention contemplates a method for generating sets of combinatorial mutants, as well as truncation mutants, and is especially useful for identifying potential agonistic or antagonistic variant sequences. The purpose of screening such combinatorial libraries is to generate, for example, novel variants which can agonize or antagonize the function of a particular gene. Such variants may be useful as agents to promote differentiation of a stem cell to a particular differentiated cell type. In one example, a variegated library of variants is generated by combinatorial mutagenesis at the nucleic acid level, and is encoded by a variegated gene library. For instance, a mixture of synthetic oligonucleotides can be enzymatically ligated into gene sequences such that the degenerate set of potential sequences are expressible as individual polypeptides, or alternatively, as a set of larger fusion proteins (e.g. for phage display) containing the set of sequences therein.

The library of potential variants can be generated from a degenerate oligonucleotide sequence using a variety of methods. Chemical synthesis of a degenerate gene sequence can be carried out in an automatic DNA synthesizer, and the synthetic genes then ligated into an appropriate expression vector. One purpose of a degenerate set of genes is to provide, in one mixture, all the sequences encoding the desired set of potential variant sequences. The synthesis of degenerate oligonucleotides is known in the art.

A range of techniques are known for screening gene products of combinatorial libraries made by point mutations, and for screening cDNA libraries for gene products having a certain property. Such techniques will be generally adaptable for rapid screening of the gene libraries generated by combinatorial mutagenesis. These techniques are also applicable for rapid screening of other gene libraries. One example of the techniques used for screening large gene libraries includes cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

The application also describes reducing a protein to generate mimetics, e.g. peptide or non-peptide agents. Mimetics having a desired biological activity can be readily tested in vitro or in vivo.

The present invention also contemplates the use of agents that are nucleic acid inhibitors such as antisense oligonucleotide, RNAi constructs, and ribozymes, as well as agents that are either protein activators of inhibitors such as small organic molecules, antibodies, and the like.

Constructs comprising the subject agents may be administered in biologically effective carriers, e.g. any formulation or composition capable of effectively delivering the agents to cells in vivo or in vitro. The particular approach can be selected from amongst those well known to one of skill in the art based on the particular agent to be delivered (e.g., nucleic acid, peptide, polypeptide, peptidomimetic, ribozyme, RNAi construct, antibody, antisense oligonucleotide, small organic molecule, and the like), the cell type to which delivery is desired, and the route of administration.

Approaches include viral vectors including recombinant retroviruses, adenovirus, adeno-associated virus, herpes simplex virus-1, lentivirus, mammalian baculovirus or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct, electroporation or CaPO₄ precipitation. One of skill in the art can readily select from available vectors and methods of delivery in order to optimize expression in a particular cell type or under particular conditions.

Retrovirus vectors and adeno-associated virus vectors have been frequently used for the transfer of exogenous genes. These vectors can be used to deliver nucleic acids, for example RNAi constructs, as well as to deliver nucleic acids encoding particular proteins. These vectors provide efficient delivery of genes into cells. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes. Thus, recombinant retrovirus can be constructed in which part of the retroviral coding sequence (gag, pol, env) has been replaced by nucleic acid encoding one of the subject proteins rendering the retrovirus replication defective. The replication defective retrovirus is then packaged into virions through the use of a helper virus by standard techniques which can be used to infect a target cell. Protocols for producing recombinant retroviruses and for infecting cells in vitro or in vivo with such viruses can be found in Current Protocols in Molecular Biology, Ausubel, F. M. et al. (eds.) Greene Publishing Associates, (2000), and other standard laboratory manuals. Examples of suitable retroviruses include pBPSTR1, pLJ, pZIP, pWE and pEM which are known to those skilled in the art. Examples of suitable packaging virus lines for preparing both ecotropic and amphotropic retroviral systems include ψCrip, ψCre, ψ2, ψAm, and PA317.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein; or coupling cell surface receptor ligands to the viral env proteins. Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences which control expression of the gene of the retroviral vector such as tetracycline repression or activation.

Another viral gene delivery system which has been employed utilizes adenovirus-derived vectors. The genome of an adenovirus can be manipulated so that it encodes and expresses a gene product of interest but is inactivated in terms of its ability to replicate in a normal lytic viral life cycle. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they can be used to infect a wide variety of cell types, including airway epithelium, endothelial cells, hepatocytes, and muscle cells. Furthermore, the virus particle is relatively stable and amenable to purification and concentration, and as above, can be modified so as to affect the spectrum of infectivity.

Yet another viral vector system is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158: 97-129). It is also one of the few viruses that may integrate its DNA into non-dividing cells, and exhibits a high frequency of stable integration.

Another viral delivery system is based on herpes simplex-1 (HSV-1). HSV-1 based vectors may be especially useful in the methods of the present invention because they have been previously shown to infect neuronal cells. Given that many adult neuronal cells are post-mitotic, and thus have been difficult to infect using some other commonly employed viruses, the use of HSV-1 represents a substantial advance and further underscores the potential utility of viral based systems to facilitate gene expression in the nervous system (Agudo et al. (2002) Human Gene Therapy 13: 665-674; Latchman (2001) Neuroscientist 7: 528-537; Goss et al. (2002) Diabetes 51: 2227-2232; Glorioso (2002) Current Opin Drug Discov Devel 5: 289-295; Evans (2002) Clin Infect Dis 35: 597-605; Whitley (2002) Journal of Clinical Invest 110: 145-151; Lilley (2001) Curr Gene Ther 1: 339-359).

The above cited examples of viral vectors are by no means exhaustive. However, they are provided to indicate that one of skill in the art may select from well known viral vectors, and select a suitable vector for expressing a particular protein in a particular cell type.

In addition to viral transfer methods, such as those illustrated above, non-viral methods can be used. Many nonviral methods of gene transfer rely on normal mechanisms used by cells for the uptake and intracellular transport of macromolecules. Exemplary gene delivery systems of this type include liposomal derived systems, poly-lysine conjugates, and artificial viral envelopes.

It may sometimes be desirable to introduce a nucleic acid directly to a cell, for example a cell in culture or a cell in an animal. Such administration can be done by injection of the nucleic acid (e.g., DNA, RNA) directly at the desired site. Such methods are commonly used in the vaccine field, specifically for administration of “DNA vaccines”, and include condensed DNA (U.S. Pat. No. 6,281,005).

In addition to administration of nucleic acids, the systems and methods described herein contemplate that polypeptides may be administered directly. Some proteins, for example factors that act extracellularly by contacting a cell surface receptor, such as growth factors, may be administered by simply contacting cells with said protein. For example, cells are typically cultured in media which is supplemented by a number of proteins such as FGF, TGFβ, insulin, etc. These proteins influence cells by simply contacting the cells. Such a method similarly pertains to other agents such as small organic molecules and chemical compounds. These agents may either exert their effect at the cell surface, or may be able to permeate the cell membrane without the need for additional manipulation.

In another embodiment, a polypeptide is directly introduced into a cell. Methods of directly introducing a polypeptide into a cell include, but are not limited to, protein transduction and protein therapy. For example, a protein transduction domain (PTD) can be fused to a nucleic acid encoding a particular polypeptide antagonist, and the fusion protein is expressed and purified. Fusion proteins containing the PTD are permeable to the cell membrane, and thus cells can be directly contacted with a fusion protein (Derossi et al. (1994) Journal of Biological Chemistry 269: 10444-10450; Han et al. (2000) Molecules and Cells 6: 728-732; Hall et al. (1996) Current Biology 6: 580-587; Theodore et al. (1995) Journal of Neuroscience 15: 7158-7167).

Although some protein transduction based methods rely on fusion of a polypeptide of interest to a sequence which mediates introduction of the protein into a cell, other protein transduction methods do not require covalent linkage of a protein of interest to a transduction domain. At least two commercially available reagents exist that mediate protein transduction without covalent modification of the protein (Chariot™, produced by Active Motif, www.activemotif.com and Bioporter® Protein Delivery Reagent, produced by Gene Therapy Systems, www.genetherapysystems.com).

Briefly, these protein transduction reagents can be used to deliver proteins, peptides and antibodies directly to cells including mammalian cells. Delivery of proteins directly to cells has a number of advantages. Firstly, many current techniques of gene delivery are based on delivery of a nucleic acid sequence which must be transcribed and/or translated by a cell before expression of the protein is achieved. This results in a time lag between delivery of the nucleic acid and expression of the protein. Direct delivery of a protein decreases this delay. Secondly, delivery of a protein often results in transient expression of the protein in a cell.

As outlined herein, protein transduction mediated by covalent attachment of a PTD to a protein can be used to deliver a protein to a cell. These methods require that individual proteins be covalently appended with PTD moieties. In contrast, methods such as Chariot™ and Bioporter® facilitate transduction by forming a noncovalent interaction between the reagent and the protein. Without being bound by theory, these reagents are thought to facilitate transit of the cell membrane, and following internalization into a cell the reagent and protein complex disassociates so that the protein is free to function in the cell.

(vi) Methods of Administration of Nucleic Acids, Proteins, Chemical Compounds and Pharmaceutical Compositions of Agents

An agent identified by the subject methods has many potential uses. Such an agent may be a nucleic acid, peptide, polypeptide, peptidomimmetic, RNAi construct, chemical compound, small organic molecule, antisense RNA, ribozyme, antibody, and the like. By agent is meant to include a single agent, or a combination of agents which together possess the desired activity. An exemplary agent promotes the differentiation of a cell (either a stem cell or a non-stem cell) to a particular differentiated cell type. In one embodiment, an agent promotes the differentiation of a cell to a neuronal cell type including, but not limited to, a dopaminergic neuron, a motor neuron, a serontenergic neuron, an interneuron, a sensory neuron, and the like. In another embodiment, an agent promotes the differentiation of a cell to a mesodermal cell type including, but not limited to, osteocytes, chondrocytes, blood cells, cells of the immune system, skeletal muscle cells, cardiac muscle cells, smooth muscle cells, cells of the kidney, and the like. In yet another embodiment, an agent promotes the differentiation of a cell to an endodermal cell type including, but not limited to, pancreatic cell types (such as β-islet cells), hepatocytes, cells of the lung, and cells of the gastrointestinal tract.

The invention contemplates the use of agents individually or in combination. Suitable combinations include combinations of multiple agents identified as promoting either progressive or terminal differentiation. Multiple agents may act additively or synergistically, and include combinations of agents that may show little or no effect when administered alone. Furthermore, the invention contemplates the use of agents in combination with known factors that influence proliferation, differentiation, or survival of a particular cell type. Still further, the invention contemplates the use of agents as part of a therapeutic regimen along with other surgical, radiological, chemical, homeopathic, or pharmacologic intervention appropriate for the particular cell type, disease or condition.

Agents which possess one of more of these characteristics may be useful in a therapeutic context. For example, injuries and diseases of the central and peripheral nervous system effect a tremendous number of people and exact a large financial and person toll. Injuries include traumatic injuries (i.e., breaks, blunt injury, burns, lacerations) to the brain or spinal cord, as well as other injuries to any region of the CNS or PNS including, but not limited to, injuries caused by bacterial infection, viral infection, cell damage following surgery, exposure to a toxic agent, cellular damage caused by cancer or other proliferative disorder, ischemia, hypoxia, and the like. Currently, effective treatments for injuries of the CNS and PNS are limited, and individuals often experience long-term deficits consistent with the extent of injury, the location of the injury, and the types of cell that are effected.

In addition to injures of the CNS and PNS, there are a wide variety of neurodegenerative diseases that effect particular regions and/or cell types of the CNS or PNS. These diseases are often progressive in nature, and individuals afflicted with many of these diseases have few treatment options at there disposal. Exemplary neurodegenerative diseases include, but are not limited to, Parkinson's disease, Huntington's disease, Alzheimer's disease, ALS, multiple sclerosis, stroke, macular degeneration, peripheral neuropathy, and diabetic neuropathy.

Given that the present invention provides methods of identifying agents that promote differentiation of cells to mesodermal and endodermal cell types, as well as neuronal cell types, agents which promote the differentiation to particular mesodermal or endodermal cell types may be used in methods of treating injuries or diseases of those tissues. Injuries and diseases of tissues derived from the mesoderm or endoderm include, but are not limited to, myocardial infarction, osteoarthritis, rheumatoid arthritis, diabetes, cirrohsis, polycystic kidney disease, inflammatory bowel disease, pancreatitis, Crohn's disease, cancer of any mesodermal or endodermal tissue (e.g, pancreatic cancer, Wilms tumor, soft cell carcinoma, bone cancer, breast cancer, prostate cancer, ovarian cancer, uterine cancer, liver cancer, colon cancer, etc), and injuries to any mesodermal or endodermal tissue including breaks, tears, bruises, lacerations, burns, toxicity, bacterial infection, and viral infection.

Furthermore, agents identified by the methods of the present invention may be used to modulate cells of the blood and blood vessels. Exemplary agents can be used to modulate (promote or inhibit) angiogenesis. Inhibition of angiogenesis is of particular use in the treatment of many forms of cancers, as well as in conditions aggravated by excess angiogenesis such as macular degeneration. Promotion of angiogenesis is of particular use in the treatment of conditions caused or aggravated by decreased blood flow. Exemplary conditions include, but are not limited to, myocardial infarction, stroke, and ischemia. Additionally, agents identified by the methods of the present invention can be used to promote proliferation and differentiation of various cell types of the blood and can be used in the treatment of anemia, leukemia, and various immunodeficiencies.

In yet another example, agents identified by the methods of the present invention can be used to modulate the differentiation of hair follicle and/or epidermal stem cells and thereby modulate hair growth.

For any of the foregoing, the application contemplates that agents may be administered alone, or may be administered in combination with other agents. Further, the application contemplates that agents identified according to the subject methods can be administered as part of a therapeutic regimen along with other treatments appropriate for the particular injury or disease being treated. For example, in the case of Parkinson's disease, a subject agent may be administered in combination with L-dopa or other Parkinson's disease medications, or in combination with a cell based neuronal transplantation therapy for Parkinson's disease. In the case of an injury to the brain or spinal cord, a subject agent may be administered in combination with physical therapy, hydrotherapy, massage therapy, and the like. In the case of peripheral neuropathy, as for example diabetic neuropathy, a subject agent may be administered in combination with insulin. In the case of myocardial infarction, the subject agent may be administered along with angioplasty, surgery, blood pressure medication, and/or as part of an exercise and diet regimen.

Exemplary Conditions Which May be Treated by the Methods of the Present Invention.

a. Injury

Physical injuries may result in cellular damage that ultimately limits the function of a particular cell or tissue. For example, physical injuries to cells in the CNS may limit the function of cells in the brain, spinal cord, or eye. Examples of physical injuries include, but are not limited to, crushing or severing of neuronal tissue, such as may occur following a fall, car accident, gun shot or stabbing wound, etc. Further examples of physical injuries include those caused by extremes in temperature such as burning, freezing, or exposure to rapid and large temperature shifts.

Physical injuries to mesodermal cell types include injuries to skeletal muscle, cardiac muscle, tendon, ligament, cartilage, bone, and the like. Examples of physical injuries include, but are not limited to, crushing, severing, breaking, bruising, and tearing of muscle tissue, bone or cartilage such as may occur following a fall, car accident, gun shot or stabbing wound, etc. Further examples of physical injuries include breaking, tearing, or bruising of muscle tissue, bone, cartilage, ligament, or tendon as may occur following a sports injury or due to aging. Further examples of physical injuries include those caused by extremes in temperature such as burning, freezing, or exposure to rapid and large temperature shifts.

Physical injuries to endodermal cell types include injuries to hepatocytes and pancreatic cell types. Examples of physical injuries include, but are not limited to, crushing, severing, and bruising, such as may occur following a fall, car accident, gun shot or stabbing wound, etc. Further examples of physical injuries include those caused by extremes in temperature such as burning, freezing, or exposure to rapid and large temperature shifts.

Further examples of an injury to any of the aforementioned cell types include those caused by infection such as by a bacterial or viral infection. Examples of bacterial or viral infections include, but are not limited to, meningitis, staph, HIV, hepatitis A, hepatitis B, hepatitis C, syphilis, human pappiloma virus, strep, etc. However, one of skill in the art will recognize that many different types of bacteria or viruses may infect cells and cause injury.

Additionally, injury to a particular cell type may occur as a consequence or side effect of other treatments being used to relieve some condition in an individual. For example, cancer treatments (chemotherapy, radiation therapy, surgery) may cause significant damage to both cancerous and healthy cells. Surgery; implantation of intraluminal devices; the placement of implants, pacemakers, shunts; and the like can all result in cellular damage.

b. Exemplary diseases

A wide range of neurodegenerative diseases cause extensive cell damage (i.e., injury) to cells of the CNS and PNS. Accordingly, neurodegenerative diseases are candidates for treatment using the described agents. Administration of a subject agent can promote neuronal regeneration in the CNS or PNS of a patient with a neurodegenerative disease, and the promotion of neuronal regeneration can ameliorate, at least in part, symptoms of the disease. Agents may be administered individually, in combination with other agents of the invention, or as part of a treatment regimen appropriate for the specific condition being treated. The following are illustrative examples of neurodegenerative conditions which can be treated using the subject agents.

Parkinson's disease is the result of the destruction of dopamine-producing neurons of the substantia nigra, and results in the degeneration of axons in the caudate nucleus and the putamen degenerate. Although therapies such as L-dopa exist to try to ameliorate the symptoms of Parkinson's disease, to date we are unaware of treatments which either prevent the degeneration of axons and/or increase neuronal regeneration. Administration of agents with promote neuronal regeneration can help to ameliorate at least certain symptoms of Parkinson's disease including rigidity, tremor, bradykinesia, poor balance and walking problems.

Alzheimer's disease, a debilitating disease characterized by amyloid plaques and neurofibrillary tangles, results in a loss of nerve cells in areas of the brain that are vital to memory and other mental abilities. There also are lower levels of chemicals in the brain that carry complex messages back and forth between nerve cells. Alzheimer's disease disrupts normal thinking and memory. The incidence of Alzheimer's disease will only increase as the average life expectancy continues to rise around the world. One of the most notable features of Alzheimer's disease is that affected individuals can live for extended periods of time (ten or more years) while being in an extremely debilitated state often requiring round the clock care. Accordingly, the disease takes not only an enormous emotional toll, but also exacts a tremendous financial toll on affected individuals and their families. Therapies which improve neuronal function have substantial utility in improving the quality of life of Alzheimer's sufferers.

Huntington's disease is a degenerative disease whose symptoms are caused by the loss of cells in a part of the brain called the basal ganglia. This cell damage affects cognitive ability (thinking, judgment, memory), movement, and emotional control. Symptoms appear gradually, usually in midlife, between the ages of 30 and 50. However, the disease can also strike young children and the elderly. Huntington's disease is a genetic disorder. Although people diagnosed with the disease can often maintain their independence for several years following diagnosis, the disease is degenerative and eventually fatal. Currently, there are no treatments available to either cure or to ameliorate the symptoms of this disease. Furthermore, the onset of Huntington's disease is typically in middle-age (approx age 40), at a time when many people have already had children. Thus, people have usually passed this fatal genetic disorder to their off-spring before they realize that they are ill.

Amyotrophic lateral sclerosis (ALS), often referred to as “Lou Gehrig's disease,” is a progressive neurodegenerative disease that attacks motor nerve cells in the brain and the spinal cord. Degeneration of motor neurons affect the ability of the brain to initiate and control muscle movement. With all voluntary muscle action affected, patients in the later stages of the disease become totally paralyzed, and eventually die.

Multiple sclerosis (MS) is an illness diagnosed in over 350,000 persons in the United States today. MS is characterized by the appearance of more than one (multiple) areas of inflammation and scarring of the myelin in the brain and spinal cord. Thus, a person with MS experiences varying degrees of neurological impairment depending on the location and extent of the scarring. The most common characteristics of MS include fatigue, weakness, spasticity, balance problems, bladder and bowel problems, numbness, vision loss, tremor and vertigo. The specific symptoms, as well as the severity of these symptoms, varies from patient to patient and is largely determined by the particular location within the brain of the lesions.

MS is considered an autoimmune disease. Recent data suggest that common viruses may play a role in the onset of MS. If so, MS may be caused by a persistent viral infection or alternatively, by an immune process initiated by a transient viral infection in the central nervous system or elsewhere in the body. Epidemiological studies indicating the distribution of MS patients suggest that there is a triggering factor responsible for initiating onset of the disease. Without being bound by theory, it appears that some environmental factor, most likely infectious, must be encountered.

The incidence of MS is higher in North America and Europe and this geographic distribution is further suggestive of an environmental influence(s) underlying onset of MS. Additionally, MS is more prevalent in women than in men, and is more common amongst Caucasians than within either Hispanic or African-American populations. Interestingly, MS is extremely rare within Asian populations.

Macular degeneration is a catch-all term for a number of different disorders that have a common end result: the light-sensing cells of the central region of the retina—the macula—malfunction and eventually die, with gradual decline and loss of central vision, while peripheral vision is retained. Most cases of macular degeneration are isolated, individual, occurrences, mostly in people over age 60. These types are called Age Related Macular Degeneration (AMD). More rarely however, younger people, including infants and young children, develop macular degeneration, and they do so in clusters within families. These types of macular degeneration are collectively called Juvenile Macular Degeneration and include Stargardt's disease, Best's vitelliform macular dystrophy, Doyne's honeycomb retinal dystrophy, Sorsby's fundus dystrophy, Malattia levintinese, Fundus flavimaculatus, and Autosomal dominant hemorrhagic macular dystrophy.

The present invention makes available effective therapeutic agents for restoring cartilage function to a connective tissue. Such methods are useful in, for example, the repair of defects or lesions in cartilage tissue which is the result of degenerative wear such as that which results in arthritis, as well as other mechanical derangements which may be caused by trauma to the tissue, such as a displacement of torn meniscus tissue, meniscectomy, a Taxation of a joint by a torn ligament, misalignment of joints, bone fracture, or by hereditary disease. The present reparative method is also useful for remodeling cartilage matrix, such as in plastic or reconstructive surgery, as well as periodontal surgery. The present method may also be applied to improving a previous reparative procedure, for example, following surgical repair of a meniscus, ligament, or cartilage. Furthermore, it may prevent the onset or exacerbation of degenerative disease if applied early enough after trauma.

Such connective tissues as articular cartilage, interarticular cartilage (menisci), costal cartilage (connecting the true ribs and the sternum), ligaments, and tendons are particularly amenable to treatment. As used herein, regenerative therapies include treatment of degenerative states which have progressed to the point of which impairment of the tissue is obviously manifest, as well as preventive treatments of tissue where degeneration is in its earliest stages or imminent. The subject method can further be used to prevent the spread of mineralisation into fibrotic tissue by maintaining a constant production of new cartilage.

In an illustrative embodiment, the subject method can be used to treat cartilage of a diarthroidal joint, such as a knee, an ankle, an elbow, a hip, a wrist, a knuckle of either a finger or toe, or a temperomandibular joint. The treatment can be directed to the meniscus of the joint, to the articular cartilage of the joint, or both. To further illustrate, the subject method can be used to treat a degenerative disorder of a knee, such as which might be the result of traumatic injury (e.g., a sports injury or excessive wear) or osteoarthritis.

In still further embodiments, agents of the present invention can be employed for the generation of bone (osteogenesis) at a site in the animal where such skeletal tissue is deficient. For instance, administration of an agent that promotes the differentiation of stem cells to bone can be employed as part of a method for treating bone loss in a subject, e.g. to prevent and/or reverse osteoporosis and other osteopenic disorders, as well as to regulate bone growth and maturation. For example, preparations comprising the identified agents can be employed, for example, to induce endochondral ossification. Therapeutic compositions can be supplemented, if required, with other osteoinductive factors, such as bone growth factors (e.g. TGF-β factors, such as the bone morphogenetic factors BMP-2 and BMP-4, as well as activin), and may also include, or be administered in combination with, an inhibitor of bone resorption such as estrogen, bisphosphonate, sodium fluoride, calcitonin, or tamoxifen, or related compounds.

The present invention further provides agents that promote differentiation of endodermal cell types, specifically definitive endodermal cell types. Such agents can be used to treat conditions associated, in whole or in part, by loss of, injury to, or decrease in functional performance of endodermal cell types. By way of example, definitive endodermal cell type include, but are not limited to, hepatocytes of the liver, pancreatic cell types such as β-islet cells, cells of the lung, and cells of the gastrointestinal tract. The following are illustrative of disease states that can be treated using agents that promote differentiation to specific endodermal cell types.

Pancreatic Diseases

1. Diabetes Mellitus

Diabetes mellitus is the name given to a group of conditions affecting about 17 million people in the United States. The conditions are linked by their inability to create and/or utilize insulin. Insulin is a hormone produced by the beta cells in the pancreas. It regulates the transportation of glucose into most of the body's cells, and works with glucagon, another pancreatic hormone, to maintain blood glucose levels within a narrow range. Most tissues in the body rely on glucose for energy production.

Diabetes disrupts the normal balance between insulin and glucose. Usually after a meal, carbohydrates are broken down into glucose and other simple sugars. This causes blood glucose levels to rise and stimulates the pancreas to release insulin into the bloodstream. Insulin allows glucose into the cells and directs excess glucose into storage, either as glycogen in the liver or as triglycerides in adipose (fat) cells. If there is insufficient or ineffective insulin, glucose levels remain high in the bloodstream. This can cause both acute and chronic problems depending on the severity of the insulin deficiency. Acutely, it can upset the body's electrolyte balance, cause dehydration as glucose is flushed out of the body with excess urination and, if unchecked, eventually lead to renal failure, loss of consciousness, and death. Over time, chronically high glucose levels can damage blood vessels, nerves, and organs throughout the body. This can lead to other serious conditions including hypertension, cardiovascular disease, circulatory problems, and neuropathy.

2. Pancreatitis

Pancreatitis can be an acute or chronic inflammation of the pancreas. Acute attacks often are characterized by severe abdominal pain that radiates from the upper stomach through to the back and can cause effects ranging from mild pancreas swelling to life-threatening organ failure. Chronic pancreatitis is a progressive condition that may involve a series of acute attacks, causing intermittent or constant pain as it permanently damages the pancreas.

Normally, the pancreatic digestive enzymes are created and carried into the duodenum (first part of the small intestine) in an inactive form. It is thought that during pancreatitis attacks, these enzymes are prevented or inhibited from reaching the duodenum, become activated while still in the pancreas, and begin to autodigest and destroy the pancreas. While the exact mechanisms of pancreatitis are not well understood, it is more frequent in men than in women and is known to be linked to and aggravated by alcoholism and gall bladder disease (gallstones that block the bile duct where it runs through the head of the pancreas and meets the pancreatic duct, just as it joins the duodenum). These two conditions are responsible for about 80% of acute pancreatitis attacks and figure prominently in chronic pancreatitis. Approximately 10% of cases of acute pancreatitis are due to idiopathic (unknown) causes. The remaining 10% of cases are due to any of the following: drugs such as valproic acid and estrogen; viral infections such as mumps, Epstein-Barr, and hepatitis A or B; hypertriglyceridemia, hyperparathyroidism, or hypercalcemia; cystic fibrosis or Reye's syndrome; pancreatic cancer; surgery in the pancreas area (such as bile duct surgery); or trauma.

Acute Pancreatitis

About 75% of acute pancreatitis attacks are considered mild, although they may cause the patient severe abdominal pain, nausea, vomiting, weakness, and jaundice. These attacks cause local inflammation, swelling, and hemorrhage that usually resolves itself with appropriate treatment and does little or no permanent damage. About 25% of the time, complications develop, such as tissue necrosis, infection, hypotension (low blood pressure), difficulty breathing, shock, and kidney or liver failure.

Chronic Pancreatitis

Patients with chronic pancreatitis may have recurring attacks with symptoms similar to those of acute pancreatitis. The attacks increase in frequency as the condition progresses. Over time, the pancreas tissue becomes increasingly scarred and the cells that produce digestive enzymes are destroyed, causing pancreatic insufficiency (inability to produce enzymes and digest fats and proteins), weight loss, malnutrition, ascities, pancreatic pseudocysts (fluid pools and destroyed tissue that can become infected), and fatty stools. As the cells that produce insulin and glucagons are destroyed, the patient may become permanently diabetic.

3. Pancreatic insufficiency

Pancreatic insufficiency is the inability of the pancreas to produce and/or transport enough digestive enzymes to break down food in the intestine and allow its absorption. It typically occurs as a result of chronic pancreatic damage caused by any of a number of conditions. It is most frequently associated with cystic fibrosis in children and with chronic pancreatitis in adults; it is less frequently but sometimes associated with pancreatic cancer.

Pancreatic insufficiency usually presents with symptoms of malabsorption, malnutrition, vitamin deficiencies, and weight loss (or inability to gain weight in children) and is often associated with steatorrhea (loose, fatty, foul-smelling stools). Diabetes also may be present in adults with pancreatic insufficiency.

Liver Diseases

1. Hepatitis

There are two major forms of hepatitis: one in which the liver is damaged quickly (called acute hepatitis) and one in which the liver is damaged slowly, over a long time (called chronic hepatitis). Hepatitis can be caused by chemicals, however, it is most commonly due to infection by one of several viruses that mainly damage the liver, termed hepatitis viruses. These viruses have been named in the order of their discovery as hepatitis A, B, C, D, and E. Hepatitis A is spread through infected water and food and is especially common in children. Most infected people don't even know they have been exposed to the virus. Hepatitis B is fairly common, especially in Asia and Africa. Although hepatitis B is less common in other parts of the world, it is still the most common cause of acute viral hepatitis in North America and Europe. Hepatitis B can be spread by exposure to blood, through sexual relations, and during pregnancy and childbirth. Symptoms of hepatitis B may be absent, mild and flu-like, or acute. Approximately 1-3% of patients become chronically infected, able to continue to infect others, and often have chronic damage to the liver. Those with weakened or compromised immune systems are at an increased risk to become carriers (about 10%). Newborns are especially vulnerable, with over 90% becoming carriers. Hepatitis C is passed the same way as hepatitis B. Hepatitis C is less common than B as a cause of acute hepatitis, but the majority of the people who contract it become chronically infected, able to spread the infection to others, and usually have chronic damage to the liver. Hepatitis D and E are rare in the United States, however, they are responsible for liver damage elsewhere in the world.

2. Cirrhosis

Anything that causes severe ongoing injury to the liver can lead to cirrhosis. It is marked by cell death and scar formation and is a progressive disease that creates irreversible damage. Cirrhosis has no signs or symptoms in its early stages, but as it progresses, it can cause fluid build-up in the abdomen (called ascites), muscle wasting, bleeding from the intestines, easy bruising, enlargement of the breasts in men (called gynecomastia), and a number of other problems.

3. Obstruction

Gallstones, tumors, trauma, and inflammation can cause blockage or obstructions in the ducts draining the liver (bile ducts). When an obstruction occurs, bile and its associated wastes accumulate in the liver and the patient's skin and eyes often turn yellow (jaundice). Bilirubin accumulating in the urine turns it a dark brown color, while lack of bilirubin in the intestines causes the stool to become very pale colored.

Obstruction of the hepatic vein, the vein from the liver, may also occur, reducing blood flow out of the liver. This obstruction may be due to tumors pushing against the vein or from blood clot formation within the vein. Obstructions may be chronic and cause few symptoms, but they can also be acute and life threatening. Some can be treated with medications; others require surgery.

4. Fatty Liver

Fatty liver causes liver enlargement, tenderness, and abnormal liver function. The most common cause is excessive alcohol consumption. Another cause of fatty liver is NASH (nonalcoholic steatohepatitis). While symptom of fatty liver are often fairly mild, the condition can lead to chronic hepatitis and cirrhosis.

5. Genetic liver disorders

Hemochromatosis is the most common genetic liver disorder. It involves excess iron storage and is usually diagnosed in adults. There are numerous genetic liver diseases that affect children. Most of the diseases involve a defective element that results in liver injury (such as biliary atresia, where the bile ducts are absent or too small) or a missing enzyme or protein that leads to damaging deposits in the liver (such as galactosemia, the absence of a milk sugar enzyme, which leads to milk sugar accumulation; and Wilson's disease, where copper builds up in the liver).

Liver disease is often discovered during routine testing. It may not cause any symptoms at first or the symptoms may be vague, like weakness and loss of energy. In acute liver disease, symptoms related to problems handling bilirubin, including jaundice (yellowing of the skin and eyes), dark urine, and light stools, along with loss of appetite, nausea, vomiting, and diarrhea are the most common. Chronic liver disease symptoms include jaundice, dark urine, abdominal swelling (due to ascites), pruritus (itching), unexplained weight loss or gain, and abdominal pain.

c. Agents that Modulate Signaling Via a Particular Signaling Pathway

The foregoing injuries and diseases are illustrative of conditions that can be treated by agents identified by the methods of the present invention. Such agents are identified based on their ability to promote progressive or terminal differentiation of a cell along a particular lineage. These agents can be identified and used without knowledge of their particular mechanism of action (e.g., without knowledge of the signaling pathways they influence). However, one of skill in the art will recognize that such agents include agents that agonize and antagonize various signal transduction pathways, and thereby promote progressive or terminal differentiation of a cell.

Additionally, however, the present invention contemplates the use of the stem cell based methods of the invention to identify, confirm, and/or characterize agents that agonize or antagonize signaling via particular signal transduction pathways. Such agonists and antagonists can be used in vitro or in vivo to modulate signal transduction via that signaling pathway, and to promote proliferation, differentiation, and/or survival or particular cell types sensitive to that signaling pathway.

By way of non-limiting example, hedgehog signaling is known to modulate the proliferation, differentiation, and survival of cells derived from all three lineages. Accordingly, hedgehog agonists and antagonists have a wide variety of uses in vitro and in vivo. Exemplary in vitro and therapeutic uses of hedgehog agonist and antagonists are provided in PCT publications WO02/30462, WO00/78374, and WO01/98344, which are hereby incorporated by reference in their entirety. Such therapeutic uses include the use of hedgehog agonists in promoting neuronal proliferation, differentiation and survival in the treatment of peripheral neuropathy, diabetic neuropathy, Parkinson's disease, Huntington's disease, macular degeneration, ALS, detached retina, Alzheimer's disease, multiple sclerosis, and stroke. Further therapeutic uses include the use of hedgehog agonists in promoting neuronal proliferation, differentiation and survival following traumatic injury to the brain or spinal cord.

Additional uses for hedgehog agonists include their use in promoting cartilage and bone repair, their use in promoting hair growth, and their use in promoting angiogenesis. Angiogenesis promoting hedgehog agonists have particular use in the treatment of ischemia and stroke.

Exemplary uses for hedgehog antagonists include their use in inhibiting angiogenesis, in inhibiting tumor growth and survival, and in inhibiting hair growth. Angiogenesis inhibiting antagonists have particular use in the treatment of a wide range of cancers and proliferative disorders affecting virtually any tissue, as well as in the treatment of macular degeneration.

Furthermore, both hedgehog agonists and antagonists are useful for influencing cell proliferation, differentiation, and survival of stem and non-stem cells in vitro and in vivo.

By way of non-limiting example, BMP signaling is known to modulate the proliferation, differentiation, and survival of cells derived from all three lineages. Accordingly, BMP agonists and antagonists have a wide variety of uses in vitro and in vivo. Exemplary in vitro and therapeutic uses of BMP agonists and antagonists are provided in PCT publications WO01/07067, WO00/61774, and U.S. Pat. No. 6,498,142, which are hereby incorporated by reference in their entirety. Such therapeutic uses include the use of BMP agonists in promoting cartilage and bone repair, their use in promoting hair growth, their use in promoting angiogenesis, and their use in promoting kidney repair.

Exemplary uses for BMP antagonists include their use in inhibiting angiogenesis, in inhibiting tumor growth and survival, in preventing pathological ossification, and in inhibiting hair growth. Angiogenesis inhibiting antagonists have particular use in the treatment of a wide range of cancers and proliferative disorders affecting virtually any tissue, as well as in the treatment of macular degeneration.

Furthermore, both BMP agonists and antagonists are useful for influencing cell proliferation, differentiation, and survival of stem and non-stem cells in vitro and in vivo.

By way of non-limiting example, Wnt signaling is known to modulate the proliferation, differentiation, and survival of cells derived from all three lineages. Accordingly, Wnt agonists and antagonists have a wide variety of uses in vitro and in vivo. Exemplary in vitro and therapeutic uses of Wnt agonists and antagonists are provided in PCT publications WO99/42481 and WO03/092719, which are hereby incorporated by reference in their entirety. Such therapeutic uses include the use of Wnt agonists in promoting proliferation of blood cells, including hematopoietic stem cells. Such Wnt agonists have particular use in the treatment of anemia, including cancer therapy or disease-induced anemia. Such Wnt agonists have additional use in the treatment of immunodeficiencies.

Exemplary uses for Wnt antagonists include their use as a cancer therapeutic, particular of cancers involving mis-regulation of the Wnt signaling pathway such as many colon cancers. Further exemplary uses of Wnt antagonists include their use in promoting adipocyte differentiation. Such Wnt antagonists have particular use in the treatment of diabetes, including Type II diabetes.

Furthermore, both Wnt agonists and antagonists are useful for influencing cell proliferation, differentiation, and survival of stem and non-stem cells in vitro and in vivo.

Agents for use in the methods of the present invention, as well as agents identified by the subject methods may be conveniently formulated for administration with a biologically acceptable medium, such as water, buffered saline, polyol (for example, glycerol, propylene glycol, liquid polyethylene glycol and the like) or suitable mixtures thereof. Optimal concentrations of the active ingredient(s) in the chosen medium can be determined empirically, according to procedures well known to medicinal chemists. As used herein, “biologically acceptable medium” includes solvents, dispersion media, and the like which may be appropriate for the desired route of administration of the one or more agents. The use of media for pharmaceutically active substances is known in the art. Except insofar as a conventional media or agent is incompatible with the activity of a particular agent or combination of agents, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Methods of introduction may also be provided by rechargeable or biodegradable devices. Various slow release polymeric devices have been developed and tested in vivo in recent years for the controlled delivery of agents, including proteinacious biopharmaceuticals. A variety of biocompatible polymers (including hydrogels), including both biodegradable and non-degradable polymers, can be used to form an implant for the sustained release of an agent at a particular target site. Delivery of agents to injury site can be attained by vascular administration via liposomal or polymeric nano-or micro-particles; slow-release vehicles implanted at the site of injury or damage; osmotic pumps implanted to deliver at the site of injury or damage; injection of agents at the site of injury or damage directly or via catheters or controlled release devices; injection into the cerebro-spinal fluid.

The agents identified using the methods of the present invention may be given orally, parenterally, or topically. They are of course given by forms suitable for each administration route. For example, they are administered in tablets or capsule form, by injection, inhalation, ointment, controlled release device or patch, or infusion.

One or more agents may be administered to humans and other animals by any suitable route of administration. With regard to administration of agents to the brain, it is known in the art that the delivery of agents to the brain may be complicated due to the blood brain barrier (BBB). Accordingly, the application contemplates that agents may be administered directly to the brain cavity. For example, agents can be administered intrathecally or intraventricularly. Administration may be, for example, by direct injection, by delivery via a catheter or osmotic pump, or by injection into the cerebrospinal fluid.

However, although the BBB may present an impediment to the delivery of agents to the brain, it is also recognized that many agents, including nucleic acids, polypeptides and small organic molecules, are able to cross the BBB following systemic delivery. Therefore, the current application contemplates that agents may be delivered either directly to the sight of injury in the CNS or PNS, or may be delivered systemically. Similarly, the invention contemplates the local delivery of agents to other sites. For example, agents can be delivered locally to the heart (e.g., intrapericardially or intramyocardially), applied topically to the skin or hair, etc.

Actual dosage levels of the one or more agents may be varied so as to obtain an amount of the active ingredient which is effective to achieve a response in an animal. The actual effective amount can be determined by one of skill in the art using routine experimentation and may vary by mode of administration. Further, the effective amount may vary according to a variety of factors include the size, age and gender of the individual being treated. Additionally the severity of the condition being treated, as well as the presence or absence of other components to the individuals treatment regimen will influence the actual dosage. The effective amount or dosage level will depend upon a variety of factors including the activity of the particular one or more agents employed, the route of administration, the time of administration, the rate of excretion of the particular agents being employed, the duration of the treatment, other drugs, compounds and/or materials used in combination with the particular agents employed, the age, sex, weight, condition, general health and prior medical history of the animal, and like factors well known in the medical arts.

The one or more agents can be administered as such or in admixtures with pharmaceutically acceptable and/or sterile carriers and can also be administered in conjunction with other compounds. These additional compounds may be administered sequentially to or simultaneously with the agents for use in the methods of the present invention. Furthermore, the one or more agents can be administered alone or in conjunction with other therapies particular for the indication being treated. Such therapies include, without limitation, other drugs therapy, surgical intervention, life-style modifications (e.g., change in diet, exercise, etc.), and homeopathic therapies (e.g., acupuncture, message, meditation, etc.).

Agents can be administered alone, or can be administered as a pharmaceutical formulation (composition). Said agents may be formulated for administration in any convenient way for use in human or veterinary medicine. In certain embodiments, the agents included in the pharmaceutical preparation may be active themselves, or may be a prodrug, e.g., capable of being converted to an active compound in a physiological setting.

Thus, another aspect of the present invention provides pharmaceutically acceptable compositions comprising an effective amount of one or more agents, formulated together with one or more pharmaceutically acceptable carriers (additives) and/or diluents. As described below, the pharmaceutical compositions of the present invention may be specially formulated for administration in solid or liquid form, including those adapted for the following: (1) local administration to the central nervous system, for example, intrathecal, intraventricular, intraspinal, or intracerebrospinal administration; (2) local administration to other tissues, for example, intramyocardial or intrapericardial administration; (3) oral administration, for example, drenches (aqueous or non-aqueous solutions or suspensions), tablets, boluses, powders, granules, pastes for application to the tongue; (4) parenteral administration, for example, by subcutaneous, intramuscular or intravenous injection as, for example, a sterile solution or suspension; (5) topical application, for example, as a cream, ointment or spray applied to the skin; or (6) opthalamic administration, for example, for administration following injury or damage to the retina. However, in certain embodiments the subject agents may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, one or more agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agent of the present invention. These salts can be prepared in situ during the final isolation and purification of the agents of the invention, or by separately reacting a purified agent of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the agents include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention. These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the agent which produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent of the present invention as an active ingredient. An agent of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the agents of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Transdermal patches have the added advantage of providing controlled delivery of an agent of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the agent in a polymer matrix or gel.

Ophthalmic formulations, eye ointments, powders, solutions and the like, are also contemplated as being within the scope of this invention. These are particularly useful for injury and degenerative disorders of the eye including retinal detachment and macular degeneration.

Pharmaceutical compositions of this invention suitable for parenteral administration comprise one or more agents of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

(vii) Exemplary Transgenic Cells and Organisms

Another aspect of the invention features transgenic non-human animals which express a heterologous gene of interest, or which have had one or more endogenous genes disrupted in at least one of the tissues or cell-types of the animal. Exemplary transgenic non-human animals include animals for use in the described screening assays, as well as animal models of injuries or diseases. Animal models of injuries and diseases can be used to test the possible in vivo therapeutic efficacy of agents identified based on their ability to promote differentiation of a cell to a particular differentiated cell type.

Another aspect of the present invention concerns transgenic animals which are comprised of cells (of that animal) which contain a transgene, as well as the cells (including stem cells) derived from these animals. In one embodiment, the expression of the transgene is restricted to specific subsets of cells, tissues or developmental stages utilizing, for example, cis-acting sequences that control expression in the desired pattern. Toward this end, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques which allow for the expression of transgenes can be regulated via site-specific genetic manipulation in vivo are known to those skilled in the art. For instance, genetic systems are available which allow for the regulated expression of a recombinase that catalyzes the genetic recombination of a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element.

Use of the cre/loxP recombinase system to regulate expression of a recombinant protein requires the construction of a transgenic animal containing transgenes encoding both the Cre recombinase and the subject protein. Animals containing both the Cre recombinase and a recombinant gene of interest can be provided through the construction of “double” transgenic animals. A convenient method for providing such animals is to mate two transgenic animals each containing a transgene.

Similar conditional transgenes can be provided using prokaryotic promoter sequences which require prokaryotic proteins to be simultaneous expressed in order to facilitate expression of a transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, a transgene could remain silent into adulthood until “turned on” by the introduction of the trans-activator.

In an exemplary embodiment, the “transgenic non-human animals” of the invention are produced by introducing transgenes into the germline of the non-human animal. Embryonic target cells at various developmental stages can be used to introduce transgenes. Different methods are used depending on the stage of development of the embryonic target cell. The zygote is the best target for micro-injection. In the mouse, the male pronucleus reaches the size of approximately 20 micrometers in diameter which allows reproducible injection of 1-2pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic non-human animal will carry the incorporated transgene. This will in general also be reflected in the efficient transmission of the transgene to offspring of the founder since 50% of the germ cells will harbor the transgene.

Retroviral infection can also be used to introduce transgenes into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonic stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474. Alternatively, the modified ES cells themselves may be used in the methods of the present invention.

Methods of making knock-out animals are also generally known. See, for example, Manipulating the Mouse Embryo, (Cold Spring Harbor Laboratory Press, Cold Spring Harbor, N.Y., 1986). Recombinase dependent knockouts can also be generated, e.g. by homologous recombination to insert recombinase target sequences flanking portions of an endogenous gene, such that tissue specific and/or temporal control of inactivation of a allele can be controlled as above.

(viii) Screening Assays and Methods of Conducting a Business

This application describes methods for identifying and/or characterizing agents that promote the differentiation of a cell to a particular differentiated cell type. Exemplary agents (e.g., a single agent, a combination of two or more agents, a library of agents) include nucleic acids, peptides, polypeptides, peptidomimmetics, antibodies, antisense RNAs, RNAi constructs (including siRNAs), ribozymes, chemical compounds, and small organic molecules. Agents may be screened individually, in combination, or as a library of agents. Furthermore, prior to contacting cells with agents of interest, cells may optionally be biased toward a particular developmental lineage by contacting the cells with one or more biasing agents. The steps of contacting the cells with biasing agents and the steps of treating cells with agents can be performed on monolayer cultures of cells and/or on cell aggregates.

Without being bound by theory, the invention contemplates that the differentiation of a cell to a particular differentiated cell type may involve the activation of particular genes and signaling pathways which promote differentiation along a particular lineage, or the inhibition of particular genes and signaling pathways which function to prevent differentiation along a particular lineage. Accordingly, the present invention contemplates screening a variety of agents such that agents can be identified based on their function (i.e., ability to promote differentiation to a particular cell type) and not based on their mechanism of action. The invention contemplates the identification of agents sufficient to promote the terminal differentiation of a cell to a particular terminally differentiated cell type. The invention further contemplates the identification of agents sufficient to promote the progressive differentiation of a cell to a cell possessing an increasing degree of commitment to a particular terminally differentiated cell type. Additionally, however, the screening methods provided herein can also be used to identify agents that both promote differentiation to a particular cell type (i.e., either progressive differentiation or terminal differentiation) and that function by agonizing or antagonizing a known signaling pathway.

The practice of any of the variety of assay methods, as exemplified herein, may identify certain agents that promote differentiation of a cell to a particular differentiated cell type. This technical step, when combined with one of more additional steps, provides pharmaceutical compositions which can be developed, tested, approved for use in humans, marketed, and sold. For example, agents according to the present invention can be tested for efficacy as therapeutics in a variety of disease models, and the potential therapeutic compositions can then be tested for toxicity and other safety-profiling before formulating, packaging and subsequently marketing the resulting formulation for the treatment of disease. Alternatively, the rights to develop and market such formulations or to conduct such steps may be licensed to a third party for consideration. In certain other aspects of the invention, the agents thus identified may have utility in the form of information that can be provided to a third party for consideration such that an improved understanding of the function or side effects of said agent in a biological or therapeutic context is obtained, or to provide an improved understanding of the cellular mechanisms that regulate cell differentiation.

In certain embodiments, the initially identified agent can be subjected to further optimization, e.g., to further refine the structure of a lead agent. Such optimization may lead to the development of analogs (e.g., modified versions of the originally identified agent) that maximize the desirable pharmacological characteristics including: solubility, permeability, bioavailability, toxicity, mutagenicity, and pharmacokinetics.

Structural modifications are made to a lead analog to address issues with the parameters listed above. These modifications however, must take into account possible effects on the analog's potency and activity. For example, if the toxicity of a lead analog is high when tested in an animal model, modifications can be made to the analog in an effort to decrease toxicity while maintaining the desired characteristic of promoting differentiation to a particular cell type.

Candidate agents (whether or not said agent is modified to make an analog of the originally identified agent possessing improved in vivo characteristics) or combinations thereof must be tested for efficacy and toxicity in animal models. Such therapeutic profiling is commonly employed in the pharmaceutical arts. Before testing an experimental therapeutic in humans, extensive therapeutic profiling (preclinical testing) must be completed to establish initial parameters for safety and efficacy. Preclinical testing establishes a mechanism of action for the therapeutic, its bioavailability, absorption, distribution, metabolism, and elimination through studies performed in vitro (that is, in test tubes, beakers, petri dishes, etc.) and in animals. Animal studies are used to assess whether the therapeutic will provide the desired results. Varying doses of the experimental therapeutic are administered to test the therapeutic's efficacy, identify harmful side-effects that may occur, and evaluate toxicity.

Briefly, one of skill in the art will recognize that the identification of a candidate agent is a first step in developing a pharmaceutical preparation useful for administration. The agent must be formulated in a pharmaceutically acceptable carrier (e.g., a pharmaceutical preparation or pharmaceutical composition). Administration of a pharmaceutical preparation comprising said agent in an amount effective to treat a condition or disease must be both safe and effective. Early stage drug trials, routinely used in the art, help to address concerns of the safety and efficacy of a potential pharmaceutical. Following initial identification of lead agents, further animal studies are necessary before initiation of human trials. Briefly, mice or rats could be administered varying doses of said pharmaceutical preparations over various time schedules. The route of administration would be appropriately selected based on the particular characteristics of the agent and on the cell type to which delivery of the agent is desired. Control mice can be administered a placebo (e.g., carrier or excipient alone).

In one embodiment, the step of therapeutic profiling includes toxicity testing of agents in cell cultures and in animals; analysis of pharmacokinetics and metabolism of the candidate agent; and determination of efficacy in animal models of relevant diseases. In certain instances, as for example when the agent is a small organic molecule, the method can include analyzing structure-activity relationship and optimizing lead analogs based on efficacy, safety and pharmacokinetic profiles. The goal of such steps is the selection of agents, or analogs of the originally identified agent, for pre-clinical studies to lead to filing of Investigational New Drug applications (“IND”) with the FDA prior to human clinical trials.

Between lead optimization and therapeutic profiling, one goal is to develop an agent that maintains the desired biological effect, and can be administered with minimal side-effects. Exemplary agents should not be exceptionally toxic (e.g., should have only tolerable side-effects when administered to patients), should not be mutagenic, and should not be carcinogenic.

By toxicity profiling is meant the evaluation of potentially harmful side-effects which may occur when an effective amount of a pharmaceutical preparation is administered. A side-effect may or may not be harmful, and the determination of whether a side effect associated with a pharmaceutical preparation is an acceptable side effect is made by the Food and Drug Administration during the regulatory approval process. This determination does not follow hard and fast rules, and that which is considered an acceptable side effect varies due to factors including: (a) the severity of the condition being treated, (b) the availability of other treatments, and (c) the side-effects associated with these currently available treatments. Presently, there are few treatment options available for individuals suffering from a spinal cord injury. Similarly, there are few treatments that provide prolonged or permanent improvements for patients suffering from Parkinson's disease, macular degeneration, Alzheimer's disease, ALS, multiple sclerosis, and many other neurodegenerative diseases. Given the paucity of treatment options for such patients, it is likely that a certain spectrum of side-effects would be considered tolerable. This is contrast to other diseases or conditions which are either not life-threatening or for which other safe and effective treatments already exist. Under these circumstances, it is likely that fewer and less severe side-effects would be considered tolerable. Nevertheless, the goal of the production of any pharmaceutical product is to minimize the number and degree of side-effects associated with administration of the pharmaceutical preparation, while maximizing the therapeutic effect of that pharmaceutical preparation.

Toxicity tests can be conducted in tandem with efficacy tests, and mice administered effective doses of the pharmaceutical preparation can be monitored for adverse reactions to the preparation.

One or more agents, or analogs thereof, which are proven safe and effective in animal studies (both non-human and human), can be formulated into a pharmaceutical preparation, and following FDA approval, readied for sale. Such pharmaceutical preparations can then be marketed, distributed, and sold. Exemplary agents may be marketed and sold alone, or may be sold as a pharmaceutical package and/or kit. Such kits include the pharmaceutical preparation along with instructions for its use. Such kits may also include devices necessary for administration of the agent such as catheters, osmotic pumps, and the like.

Furthermore, in any of the foregoing aspects, the invention appreciates that a method of providing a pharmaceutical preparation does not necessarily end with the formulation and sale of a pharmaceutical product. Such a method may also include a system for billing a patient and/or a patient's insurance provider, as well as a system for collecting appropriate reimbursement from the patient and/or the patient's insurance provider.

Exemplification

The invention now being generally described, it will be more readily understood by reference to the following examples which are included merely for purposes of illustration of certain aspects and embodiments of the present invention, and are not intended to limit the invention.

### EXAMPLE 1

### Methods of Identifying Agents that Promote Differentiation of a Embryonic Stem Cell to a Particular Differentiated Neuronal Cell Type

The following method is indicative of that which can be used to identify and/or characterize an agent that promotes the differentiation of embryonic stem cells to a particular neuronal cell type. Briefly, embryonic stem cells are cultured under standard conditions well known in the art for embryonic stem cells derived from a variety of organisms (see, for example, Wichterle et al. and Benvenisty et al.). ES cells are aggregated to form embryoid bodies. The ES cells are biased to differentiate along a neuronal fate by treatment of the embryoid bodies with retinoic acid (i.e., the ES cells are neuralized). Cells treated with retinoic acid are then contacted with one or more test agents (the cells can be contacted with the test agent either simulataneously with retinoic acid or following treatment with retinoic acid). The ability of the test agent to promote the differentiation of the embryonic stem cells to a particular neuronal cell type is assessed by examining markers of neuronal differentiation. For example, the ability of an agent to promote the terminal differentiation of an embryonic stem cell to a motor neuron can be assessed by assaying expression of HB9, the ability of the agent to promote the terminal differentiation of an embryonic stem cell to a dopaminergic neuron can be assessed by assaying the expression of tyrosine hydroxylase, and the ability of the agent to promote terminal differentiation of an embryonic stem cell to an interneuron can be assessed by assaying the expression of Math1. Further markers of terminally differentiated motor neurons include Isl1, Lhx3, and Lim1. One of skill in the art can readily select from amongst known markers of terminal differentiation of a particular neuronal cell type and readily choose one or more appropriate markers of terminal differentiation.

In addition to assessing the ability of an agent to promote the terminal differentiation of an embryonic stem cell, the ability of the agent to promote the further commitment of an embryonic stem cell to a particular neuronal cell fate can be assessed. Such agents would promote differentiation, but may or may not promote terminal differentiation. For example, prior to terminal differentiation, spinal motor neuron progenitor cells express Pax6, Nkx6.1, Olig2, but do not express Pax7, Irx3, Dbx1, and Nkx2.2. Additionally markers indicative of neuronal commitment (but not necessarily terminal differentiation) include NeuN, GFAP, peripherin, NCAM, nestin, Otx2, β-tubulin, and Sox1.

### EXAMPLE 2

### Methods of Identifying Agents that Promote Differentiation of a Stem Cell to a Particular Differentiated Neuronal Cell Type

The following method is indicative of that which can be used to identify and/or characterize agents that promote the differentiation of a stem cell to a particular neuronal cell type. Neuronal stem cells isolated from the brain of fetal or adult rats or mice are cultured according to methods well known in the art and described herein (see, for example, U.S. Pat. No. 5,411,883 and U.S. Pat. No. 6,294,346). Neuronal stem cells are aggregated to form neurospheres. The neuronal stem cells and neurospheres are already “neuralized”, and thus the step of contacting the cells with one or more factors that bias the cells to a neuronal lineage is not necessarily required. Accordingly, the cells are optionally cultured in the presence of retinoic acid, or another factor that typically biases cells to a neuronal cell fate. Biased cells (either treated with retinoic acid or not) are then contacted with one or more test agents (the cells can be contacted with the test agent either simulataneously with retinoic acid or following treatment with retinoic acid). The ability of the test agent to promote the differentiation of the stem cells to a particular neuronal cell type is assessed by examining markers of neuronal differentiation. For example, the ability of an agent to promote the terminal differentiation of an embryonic stem cell to a motor neuron can be assessed by assaying expression of HB9, the ability of the agent to promote the terminal differentiation of an embryonic stem cell to a dopaminergic neuron can be assessed by assaying the expression of tyrosine hydroxylase, and the ability of the agent to promote terminal differentiation of an embryonic stem cell to an interneuron can be assessed by assaying the expression of Math1. Further markers of terminally differentiated motor neurons include Isl1, Lhx3, and Lim1. One of skill in the art can readily select from amongst known markers of terminal differentiation of a particular neuronal cell type and readily choose one or more appropriate markers of terminal differentiation.

In addition to assessing the ability of an agent to promote the terminal differentiation of a neural stem cell, the ability of the agent to promote the further commitment of a neural stem cell to a particular neuronal cell fate can be assessed. Such agents would promote differentiation, but may or may not promote terminal differentiation. For example, prior to terminal differentiation, spinal motor neuron progenitor cells express Pax6, Nkx6.1, Olig2, but do not express Pax7, Irx3, Dbx1, and Nkx2.2. Additionally markers indicative of neuronal commitment (but not necessarily terminal differentiation) include NeuN, GFAP, peripherin, NCAM, nestin, Otx2, β-tubulin, and Sox1.

### EXAMPLE 3

### Methods of Identifying Agents that Promote Differentiation of an Embryonic Stem Cell to a Particular Differentiated Mesodermal Cell Type

The following method is indicative of a method that can be used to identify and/or characterize agents that promote the differentiation of an embryonic stem cell to a particular differentiated cell type. Briefly, embryonic stem cells are cultured under standard conditions well known in the art for embryonic stem cells derived from a variety of organisms (see, for example, Wichterle et al. and Benvenisty et al.). ES cells are aggregated to form embryoid bodies. The ES cells are biased to differentiate along a mesodermal fate by treatment of the embryoid bodies with a biasing factor. High serum is an example of a factor known to bias certain stem cell populations along mesodermal lineages. Biased cells are then contacted with one or more test agents (the cells can be contacted with the test agent either simulataneously with or following treatment with the biasing factor). The ability of the test agent to promote the differentiation of the embryonic stem cells to a particular mesodermal cell type is assessed by examining markers of mesodermal differentiation.

For example, the ability of an agent to promote the terminal differentiation of an embryonic stem cell to a skeletal muscle cell can be assessed by examining expression of myosin heavy chain, myosin light chain, troponin, and the like. The ability of an agent to promote the terminal differentiation of an embryonic stem cell to a cardiac muscle cell can be assessed by examining expression of cardiac troponin, cardiac actin, troponinT, ventricular myosin, and the like. The ability of an agent to promote the terminal differentiation of an embryonic stem cell to an adipocyte can be examined using an assay for lipid deposition. The ability of an agent to promote the terminal differentiation of an embryonic stem cell to bone can be examined using calcium deposition or AlizarinRed staining. The ability of an agent to promote the terminal differentiation of an embryonic stem cell to cartilage can be examined using AlcianBlue.

In addition to assessing the ability of an agent to promote the terminal differentiation of an embryonic stem cell, the ability of the agent to promote the commitment of an embryonic stem cell to a particular mesodermal cell fate can be assessed. Such agents would promote differentiation, but may or may not promote terminal differentiation. For example, prior to terminal differentiation, various mesodermal cell types express GATA-4, Nkx2.5, Nkx2.3, MyoD, Myf5, desmin, Indian hedgehog, parathyroid hormone, parathyroid hormone receptor, WT-1, Pax-2, Pax-8, and the like.

### EXAMPLE 4

### Methods of Identifying Agents that Promote Differentiation of a Stem Cell to a Particular Differentiated Mesodermal Cell Type

The following method is indicative of a method that can be used to identify and/or characterize agents that promote the differentiation of an adult stem cell to a particular differentiated mesodermal cell type. Adult stem cells known to differentiate to the mesodermal cell fate of interest are particularly preferred for use in this aspect of the present invention. By way of example, mesenchymal stem cells are particularly useful for screening to identify agents that promote differentiation to chondrocytes, osteocytes, adipocytes, blood, skeletal muscle and cardiac muscle. By way of further example, cardiac stem cells are particularly useful for screening to identify agents that promote cardiac differentiation. By way of still further example, stem cells derived from the kidney are particularly useful for screening to identify agents that promote differentiation of renal cell type (i.e., glomerular cells, ductal cells, tubule cells, podocytes, etc.).

Briefly, adult stem cells are cultured under standard conditions appropriate for the particular cell type being used. Although these adult stem cells are already biased to some extent, the cells may optionally be treated with one or more biasing factors to further prime the stem cells to differentiate along a particular lineage in response to one or more agents. Cells (either biased cells or cultures of unbiased cells) are then contacted with one or more test agents (the cells can be contacted with the test agent either simultaneously with or following treatment with the biasing factor). The ability of the test agent to promote the differentiation of the stem cells to a particular mesodermal cell type is assessed by examining markers of mesodermal differentiation. In any of the foregoing, the adult stem cells can be screened as a monolayer culture, or they may be aggregated to form meso-spheres (i.e., aggregates of cells that help promote differentiation).

For example, the ability of an agent to promote the terminal differentiation of an adult stem cell to a skeletal muscle cell can be assessed by examining expression of myosin heavy chain, myosin light chain, troponin, and the like. The ability of an agent to promote the terminal differentiation of an adult stem cell to a cardiac muscle cell can be assessed by examining expression of cardiac troponin, cardiac actin, troponinT, ventricular myosin, and the like. The ability of an agent to promote the terminal differentiation of an adult stem cell to an adipocyte can be examined using an assay for lipid deposition. The ability of an agent to promote the terminal differentiation of an adult stem cell to bone can be examined using calcium deposition or AlizarinRed staining. The ability of an agent to promote the terminal differentiation of an adult stem cell to cartilage can be examined using AlcianBlue.

In addition to assessing the ability of an agent to promote the terminal differentiation of an adult stem cell, the ability of the agent to promote the commitment of an adult stem cell to a particular mesodermal cell fate can be assessed. Such agents would promote differentiation, but may or may not promote terminal differentiation. For example, prior to terminal differentiation, various mesodermal cell types express GATA-4, Nkx2.5, Nkx2.3, MyoD, Myf5, desmin, Indian hedgehog, parathyroid hormone, parathyroid hormone receptor, WT-1, Pax-2, Pax-8, and the like.

### EXAMPLE 5

### Methods of Identifying Agents that Promote Differentiation of an Embryonic Stem Cell to a Particular Differentiated Endodermal Cell Type

The following method is indicative of a method that can be used to identify and/or characterize agents that promote the differentiation of an embryonic stem cell to a particular differentiated cell type. Briefly, embryonic stem cells are cultured under standard conditions well known in the art for embryonic stem cells derived from a variety of organisms (see, for example, Wichterle et al. and Benvenisty et al.). ES cells are aggregated to form embryoid bodies. The ES cells are biased to differentiate along a endoderm fate by treatment of the embryoid bodies with a biasing factor. An exemplary biasing factor known to influence the differentiation of stem cells along an endodermal lineage is nicotinamide. Commitment to differentiate along an endodermal lineage can be assessed by expression of one or more early endodermal markers in all or a portion of the biased stem cell culture. Exemplary early endodermal markers include, but are not limited to, Pdx, Sox17, Foxa2/HNF3β, mix, mixer, mix-like, HesX1, dkk1, Lim1, Cerberus, GATA4, GATA6, and HNF4. Biased cells are then contacted with one or more test agents (the cells can be contacted with the test agent either simultaneously with or following treatment with the biasing factor). The ability of the test agent to promote the differentiation of the embryonic stem cells to a particular endodermal cell type is assessed by examining markers of endodermal differentiation. For example, the ability of an agent to promote the terminal differentiation of an embryonic stem cell to a pancreatic cell can be assessed by examining markers of any of the cell types of the pancreas such as the α, β, or γ cells. Exemplary markers include insulin, glucagon, somatostatin, carboxypeptidase, or PP. The ability of an agent to promote the terminal differentiation of an embryonic stem cell to a hepatocyte can be assessed by examining expression of HNF3β, TTR, alpha fetal protein, albumin, AAT, TAT, and CPS1. The ability of an agent to promote the terminal differentiation of an embryonic stem cell to an intestinal cell can be assessed by examining expression of IFABP.

In addition to assessing the ability of an agent to promote the terminal differentiation of an embryonic stem cell, the ability of the agent to promote the commitment of an embryonic stem cell to a particular endodermal cell fate can be assessed. Such agents would promote differentiation, but may or may not promote terminal differentiation.

### EXAMPLE 6

### Methods of Identifying Agents that Promote Differentiation of a Stem Cell to a Particular Differentiated Endodermal Cell Type

The following method is indicative of a method that can be used to identify and/or characterize agents that promote the differentiation of an adult stem cell to a particular differentiated endodermal cell type. Adult stem cells known to differentiate to the endodermal cell fate of interest are particularly preferred for use in this aspect of the present invention. By way of example, pancreatic stem cells are particularly useful for screening to identify agents that promote differentiation to pancreatic and hepatic cell types. By way of further example, hepatic stem cells are particularly useful for screening to identify agents that promote differentiation of hepatic cell types. By way of still further example, stem cells derived from the gastrointestinal tract are particularly useful for screening to identify agents that promote differentiation to cell types of the stomach, small intestine, large intestine, and pancreas.

The following method is indicative of a method that can be used to identify and/or characterize agents that promote the differentiation of an adult stem cell to a particular differentiated cell type. Briefly, adult stem cells are cultured under standard conditions appropriate for the particular cell type being used. Although these adult stem cells are already biased to some extent, the cells may optionally be treated with one or more biasing factors to further prime the stem cells to differentiate along a particular lineage in response to one or more agents. Cells (either biased cells or cultures of unbiased cells) are then contacted with one or more test agents (the cells can be contacted with the test agent either simultaneously with or following treatment with the biasing factor). The ability of the test agent to promote the differentiation of the stem cells to a particular endodermal cell type is assessed by examining markers of endodermal differentiation. In any of the foregoing, the adult stem cells can be screened as a monolayer culture, or they may be aggregated to form endo-spheres (i.e., aggregates of cells that help promote differentiation).

Commitment to differentiate along an endodermal lineage can be assessed by expression of one or more early endodermal marker in all or a portion of the stem cell culture. Exemplary early endodermal markers include, but are not limited to, Pdx, Sox17, Foxa2/HNF3β, mix, mixer, mix-like, HesX1, dkk1, Lim1, Cerberus, GATA4, GATA6, and HNF4. Biased cells are then contacted with one or more test agents (the cells can be contacted with the test agent either simultaneously with or following treatment with the biasing factor). The ability of the test agent to promote the differentiation of the embryonic stem cells to a particular endodermal cell type is assessed by examining markers of endodermal differentiation. For example, the ability of an agent to promote the terminal differentiation of a stem cell to a pancreatic cell can be assessed by examining markers of any of the cell types of the pancreas such as the α, β, or γ cells. Exemplary markers include insulin, glucagon, somatostatin, carboxypeptidase, or PP. The ability of an agent to promote the terminal differentiation of a stem cell to a hepatocyte can be assessed by examining expression of HNF3β, TTR, alpha fetal protein, albumin, AAT, TAT, and CPS1. The ability of an agent to promote the terminal differentiation of a stem cell to an intestinal cell can be assessed by examining expression of IFABP.

In addition to assessing the ability of an agent to promote the terminal differentiation of a stem cell, the ability of the agent to promote the commitment of a stem cell to a particular endodermal cell fate can be assessed. Such agents would promote differentiation, but may or may not promote terminal differentiation.

### EXAMPLE 7

### Confirmation Assays Using Stem Cells

The methods of the present invention can be used to identify and/or characterize agents that promote differentiation of a stem cell to a particular differentiated cell type. The application further contemplates the use of stem cell based assays to confirm that agents identified using other cell free or cell-based assays promote differentiation to a particular lineage. Agents whose effectiveness is confirmed in this manner are candidate agents for use as a therapeutic.

Many cell free and cell based screens exist in the art to screen agents. However, many of these assays are based not on a desired physiological output, but rather based on a mechanistic output. For example, a cell free or cell based assay may be based on the ability of an agent to bind to a particular protein, phosphorylate a particular protein, dephosphorylate a particular protein, or activate signal transduction through a particular signaling pathway. Although these assays are extremely useful, especially as primary screens, the physiological relevance of agents so identified may be hard to predict.

In the present context, the stem cell-based assays described herein serve as a secondary screen to assess the physiological relevance of agents identified using other cell free or cell based assays. For example, the ability of a candidate agent previously identified using a cell free or cell based assay can be tested for the ability to promote differentiation of a stem cell to a particular differentiated cell type. A culture of embryonic stem cells is provided. Embryoid bodies are formed from the culture of embryonic stem cells, and these embryoid bodies are contacted with a preparation comprising retinoic acid, or with another factor that biases the cells to a neuronal lineage. The biased embryonic stem cells are then contacted with the candidate agent, and the ability of the candidate agent to promote the differentiation of the biased stem cells to a particular neuronal cell type is assessed. The ability of the candidate agent to promote differentiation of the biased cells to any of a number of neuronal cell types can be simultaneously examined by examining markers of several different differentiated neuronal cell types such as dopaminergic neurons, motor neurons, sensory neurons, interneurons, oligodendrocytes, astrocytes, Schwann cells, and the like.

A further example of this method of confirming the effect of an agent identified in a cell free or cell based screen is described by Frank-Kamenetsky et al. (Frank-Kamenetsky et al. (2002) Journal of Biology 1: 10). Briefly, Frank-Kamenetsky et al. describes the further characterization of a small molecule agent identified using a cell-based screen. The cell based screen assessed the ability of agents to activate a reporter construct indicative of hedgehog signaling, and thus identified agents that agonize hedgehog signaling. However, the ability of the putative hedgehog agonist to alter the differentiation of a particular cell type was not evaluated in the original screen.

To confirm the ability of the identified agent (in this case a hedgehog agonist) to promote the differentiation of a progenitor cell population, the ability of the agent to promote differentiation of primary cerebellar neurons derived from one week rat brains was assessed. Additionally, the ability of the agent to promote differentiation of embryonic chick neural tube explants was assessed. In the foregoing examples, the ability of the agent to promote differentiation was assessed by examining expression of three proteins which mark cells committed to a particular neuronal cell type: Pax7, MNR2, and Nkx2.2. Although these are not markers of terminal differentiation, they are indicative of cells which have begun to differentiate along a particular neuronal cell fate. If desired, markers of terminal differentiation to identify, for example, dopaminergic neurons, sensory neurons, motor neurons, interneuorns, astrocytes, Schwann cells, oligodendrocytes, and the like could be employed.

Of additional note, the methods provided by Frank-Kamenetsky et al. demonstrate that differentiation can be monitored in any of a number of ways. For example, a marker of a terminally differentiated cell type or a committed cell type can be assayed by immunocytochemistry (as in Frank-Kamenetsky et al.) or by Western blot analysis using an antibody immunoreactive with the particular protein. Expression of a particular marker could be assayed at the RNA level by in situ hybridization, RT-PCR, RNAse protection, GeneChip analysis, or Northern blot analysis. A still further way to examine expression of a particular marker is via the use of cells derived from transgenic animals (as, for example, in Frank-Kamenetsky et al.). Furthermore, any of these methods can be combined to assay multiple markers (i.e., multi-plex analysis).

However, the use of the stem cell based screening methods to identify agents that regulate differentiation via a particular signaling pathway is not limited to a secondary screen to confirm the function of previously identified agents. Although the stem cell based screening methods are particularly useful because they are not biased to identify agents that function via only one particular mechanism or that regulate only particular signaling pathways, such methods can still be used to identify (in a single screen) agents that both promote differentiation to a particular cell type and that modulate a particular signaling pathway.

### EXAMPLE 8

### Step-Wise Method of Identifying Agents that Promote Differentiation of an Embryonic Stem Cell to a Particular Neuronal Cell Type

It is readily appreciated in the art that the developmental pathway from a stem cell to a terminally differentiated cell type is a long path. Along the way, a cell passes through various cell types which are committed to varying degrees. Given this pathway from a stem cell to a terminally differentiated cell, it is possible that a single factor may prove insufficient to influence the development of a cell from a stem cell all the way to a particular terminally differentiated cell type. What may be more likely is that individual factors or small numbers of factors will be sufficient to influence discrete steps in the pathway from a stem cell to cell types with an increasing degree of commitment, and eventually to a particular terminally differentiated cell type.

The following method is indicative of a method that can be used to identify and/or characterize agents that promote each of a number of steps along the pathway from a stem cell to a terminally differentiated cell type. The particular markers used to track the progression of the cell from a stem cell to an increasingly committed cell, and finally to a terminally differentiated cell depend upon the particular differentiated cell type. The foregoing example provides an illustrative example in which the goal is the identification of agents that influence the progressive commitment of an embryonic stem cell to a terminally differentiated motor neuron. However, the invention contemplates that similar methodology can be employed to identify agents that influence the progressive commitment of embryonic stem cells to any of a number of terminally differentiated cell types derived from the ectoderm, mesoderm, or endoderm. One of skill in the art can readily select appropriate markers in order to following the progressive commitment of a stem cell along the ectodermal, mesodermal or endodermal lineage, and eventually to a particular terminally differentiated cell type.

Briefly, embryonic stem cells are cultured under standard conditions well known in the art. ES cells are aggregated to form embryoid bodies and the embryoid bodies are contacted with one or more agents (i.e., a single agent, a combination of agents, or a library of agents). Following this first step of contacting EBs with agents (contact 1), embryoid bodies are assayed for expression of a marker indicative of early commitment to a neuronal lineage. An exemplary marker is nestin. Accordingly, this first screening step would allow the identification of agents that promote the commitment of embryonic stem cells along a neuronal lineage as measured by expression of an early neuronal marker such as nestin.

In a second step, these committed embryoid bodies (such as nestin+ embryoid bodies) are then contacted with agents (contact 2). Following this second step of contacting EBs with agents, embryoid bodies are assayed for expression of a marker consistent with further commitment along a neuronal lineage. Accordingly, this second screening step would allow the identification of agents that promote the further commitment of embryonic stem cells along a neuronal lineage.

In a third step, these committed embryoid bodies are contacted with agents (contact 3). Following this third step of contacting EBs with agents, embryoid bodies are assayed for expression of a marker consistent with further commitment along a neuronal lineage. For example, in this third step EBs can be assayed for expression of a marker consistent with commitment to a motor neuron fate such as Pax6, Nkx6.1, and/or Olig2. Accordingly, this third screening step would allow the identification of agents that promote the further commitment of embryonic stem cells along a motor neuron fate.

In a fourth step, embryoid bodies committed to a motor neuron fate are contacted with agents (contact 4). Following this fourth step of contacting EBs with agents, embryoid bodies are assayed for expression of a marker consistent with terminal motor neuron differentiation. For example, in this fourth step EBs can be assayed for expression of a marker consistent with terminal differentiation such as HB9. Accordingly, this fourth screening step would allow the identification of agents that promote the terminal differentiation of embryonic stem cells to motor neurons.

In the foregoing example, one or more markers are analyzed at each step of the differentiation process. However, the invention contemplates a number of different methods to evaluate marker expression following contacting the cells with agents. For example, rather than predict the level of differentiation likely achieved following exposure of cells to particular agents, the invention contemplates that at each step multiple markers of various levels of commitment are analyzed. For example, at each step, early markers, intermediate markers, late markers, and markers of terminal differentiation are examined. In this way, the opportunity exists along each step of the screening method to identify and characterize agents that influence the commitment of stem cells along a particular lineage.

Of additional note, the methods examples provided by Frank-Kamenetsky et al. demonstrate that differentiation can be monitored in any of a number of ways. For example, a marker of a terminally differentiated cell type or a committed cell type can be assayed by immunocytochemistry (as in Frank-Kamenetsky et al.) or by Western blot analysis using an antibody immunoreactive with the particular protein. Expression of a particular marker could be assayed at the RNA level by in situ hybridization, RT-PCR, RNAse protection, GeneChip analysis, or Northern blot analysis. A still further way to examine expression of a particular marker is via the use of cells derived from transgenic animals (as, for example, in Frank-Kamenetsky et al.). Furthermore, any of these methods can be combined to assay multiple markers (i.e., multi-plex analysis).

Although any of the foregoing methods can be used in assaying marker expression at various time points as cells progress from a stem cell to a terminally differentiated cell, certain methods are of particular note because they further facilitate examining a single cell or group of cells following multiple rounds of exposure to agents. For example, although immunocytochemistry can be used to monitor protein expression in a cell or group of cells following contacting of those cells with an agent, those same cells will then be unavailable for use in a second round of exposure to agents. Although this shortcoming can be circumvented, certain methods would allow the examination of gene expression without the need to harvest/kill individual cells.

The use of cells derived from transgenic embryos which express one or more detectable markers under the control of the promoter of particular genes would be advantageous in this method. For example, the above outlined experiment could be performed using embryonic stem cells derived from a transgenic animal. The transgenic animal could contain several reporter constructs under the control of promoters of genes associated with varying stages of neuronal commitment and motor neuron differentiation. The cells can contain GFP under the control of a nestin promoter, YFP under the control of an Olig promoter, and RFP under the control of the HB9 promoter.

In the foregoing example of a step-wise approach to identifying agents that promote progressive differentiation of a cell to a particular differentiated cell type, the invention contemplates that at each step of contacting cells with agents (i.e., contact 1, 2, 3, etc) the process of contacting the cells is performed on all of the cells in culture. However, the invention further contemplates that at each step, prior to the next step of contacting cells with agents, particular cells which have responded to agents to become increasingly committed are separated/purified from the other cells which have not responded to the agents. In this embodiment, only cells which have responded to exposure to agents are used in subsequent rounds of analysis.

### EXAMPLE 9

### Step-Wise Method of Identifying Agents that Promote Differentiation of a Cell to a Particular Neuronal Cell Type

As detailed in example 8, the present invention provides a step-wise method of identifying agents that promote differentiation of a stem cell to a particular differentiated cell type. However, it is further appreciated that the starting cell in such a step-wise method need not be a stem cell. The input cell for “contact 1” can be a cell that is not a stem cell and has thus already received certain developmental information biasing that cell to differentiate along a particular pathway. For example, a stem cell that has been contacted with retinoic acid is already biased to a neuronal cell fate. Such a biased cell can be the input cell for contact 1 is a method to identify agents which influence each of the remaining steps taken by that biased cell in progressing to a terminally differentiated cell such as a terminally differentiated motor neuron or dopaminergic neuron.

The following method is indicative of a method that can be used to identify and/or characterize agents that promote each of a number of steps along the pathway from a biased cell to a terminally differentiated cell type. The particular markers used to track the progression of the cell from a biased cell to an increasingly committed cell, and finally to a terminally differentiated cell depend upon the particular differentiated cell type. The foregoing example provides an illustrative example in which the goal is the identification of agents that influence the progressive commitment of a cell biased along the neuronal lineage to a terminally differentiated motor neuron. However, the invention contemplates that similar methodology can be employed to identify agents that influence the progressive commitment of cell to any of a number of terminally differentiated cell types derived from the ectoderm, mesoderm, or endoderm. One of skill in the art can readily select appropriate markers in order to follow the progressive commitment of a stem cell along the ectodermal, mesodermal or endodermal lineage, and eventually to a particular terminally differentiated cell type.

Briefly, embryonic stem cells are cultured under standard conditions well known in the art. ES cells are aggregated to form embryoid bodies and the embryoid bodies are contacted with retinoic acid to bias them along a neuronal lineage. The biased cells are the input material for the first step of contact with test agents. Following this first step of contacting biased cells with agents (contact 1), cells are assayed for expression of a marker indicative of further commitment to a neuronal lineage. Accordingly, this first screening step would allow the identification of agents that promote the further commitment of cells along a neuronal lineage.

In a second step, these committed cells are then contacted with agents (contact 2). Following this second step of contacting cells with agents, cells are assayed for expression of a marker consistent with further commitment along a neuronal lineage. Accordingly, this second screening step would allow the identification of agents that promote the still further commitment of cells along a neuronal lineage and perhaps even promote commitment to a particular neuronal cell fate such as a motor neuron fate. Accordingly, this third screening step would allow the identification of agents that promote the further commitment of embryonic stem cells along a motor neuron fate.

In a third step, cells committed to a motor neuron fate are contacted with agents (contact 3). Following this third step of contacting cells with agents, cells are assayed for expression of a marker consistent with terminal motor neuron differentiation. For example, in this third step cells can be assayed for expression of a marker consistent with terminal differentiation such as HB9. Accordingly, this third screening step would allow the identification of agents that promote the terminal differentiation of cells to motor neurons.

In the foregoing example, one or more markers are analyzed at each step of the differentiation process. However, the invention contemplates a number of different methods to evaluate marker expression following contacting the cells with agents. For example, rather than predict the level of differentiation likely achieved following exposure of cells to particular agents, the invention contemplates that at each step multiple markers of various levels of commitment are analyzed. For example, at each step, early markers, intermediate markers, late markers, and markers of terminal differentiation are examined. In this way, the opportunity exists along each step of the screening method to identify and characterize agents that influence the commitment of cells along a particular lineage.

Of additional note, the methods examples provided by Frank-Kamenetsky et al. demonstrate that differentiation can be monitored in any of a number of ways. For example, a marker of a terminally differentiated cell type or a committed cell type can be assayed by immunocytochemistry (as in Frank-Kamenetsky et al.) or by Western blot analysis using an antibody immunoreactive with the particular protein. Expression of a particular marker could be assayed at the RNA level by in situ hybridization, RT-PCR, RNAse protection, GeneChip analysis, or Northern blot analysis. A still further way to examine expression of a particular marker is via the use of cells derived from transgenic animals (as, for example, in Frank-Kamenetsky et al.). Furthermore, any of these methods can be combined to assay multiple markers (i.e., multi-plex analysis).

Although any of the foregoing methods can be used in assaying marker expression at various time points as cells progress from a cell to a terminally differentiated cell, certain methods are of particular note because they further facilitate examining a single cell or group of cells following multiple rounds of exposure to agents. For example, although immunocytochemistry can be used to monitor protein expression in a cell or group of cells following contacting of those cells with an agent, those same cells will then be unavailable for use in a second round of exposure to agents. Although this shortcoming can be circumvented, certain methods would allow the examination of gene expression without the need to harvest/kill individual cells.

The use of cells derived from transgenic embryos which express one or more detectable markers under the control of the promoter of particular genes would be advantageous in this method. For example, the above outlined experiment could be performed using cells derived from a transgenic animal. The transgenic animal could contain several reporter constructs under the control of promoters of genes associated with varying stages of neuronal commitment and motor neuron differentiation. The cells can contain GFP under the control of a nestin promoter, YFP under the control of an Olig promoter, and RFP under the control of the HB9 promoter.

In the foregoing example of a step-wise approach to identifying agents that promote progressive differentiation of a cell to a particular differentiated cell type, the invention contemplates that at each step of contacting cells with agents (i.e., contact 1, 2, 3, etc) the process of contacting the cells is performed on all of the cells in culture. However, the invention further contemplates that at each step, prior to the next step of contacting cells with agents, particular cells which have responded to agents to become increasingly committed are separated/purified from the other cells which have not responded to the agents. In this embodiment, only cells which have responded to exposure to agents are used in subsequent rounds of analysis.

### EXAMPLE 10

### Neuronal Differentiation of Embryonic Stem Cells

Neuronal differentiation of embryonic stem cells recapitulates differentiation observed during development in the neural tube. This suggests that agents identified ex vivo in stem-cell based assays will also be physiologically relevant for use in vivo. An additional advantage of the similarities between differentiation of the neural tube and neuronal differentiation of embryonic stem cells is that it allows predictions of the mechanisms (i.e., via agonizing or antagonizing particular signal transduction pathways) by which agents that promote differentiation to particular cell types may function. Although the ability to make such predictions are not necessary to practice the methods of the present invention, where such predictions are possible they permit identification of both agents that promote progressive or terminal differentiation to a particular cell type, as well as agents that agonize or antagonize a particular signaling pathway.

Hedgehog signaling, BMP signaling, and Wnt signaling influence differentiation in the developing neural tube. Briefly, Wnt signaling and BMP signaling play important roles in promoting differentiation of dorsal cell types in the neural type. Exemplary dorsal cell types are dorsal interneurons, and the differentiation of dorsal interneurons can be assessed by expression of the basic helix-loop-helix (bHLH) transcription factor Math1 (Ben-Arie et al. (1997) Nature 390: 169-172; Helms and Johnson (1998) Development 125: 919-928). Hedgehog signaling plays an important role in promoting differentiation of ventral cell types in the neural tube. Exemplary ventral cells types are motor neurons, and the differentiation of motor neurons can be assessed by expression of HB9.

The known role of these three signaling pathways in patterning dorsal and ventral cell fates in the developing neural tube suggests that subsets of agents that promote progressive or terminal differentiation of stem cell will include agents that agonize or antagonize these signaling pathways. For example, important classes of agents that promote interneuron differentiation (i.e., agents that promote expression of Math1) include BMP agonists (agents that promote BMP signal transduction), Wnt agonists (agents that promote Wnt signal transduction), and hedgehog antagonists (agents that inhibit hedgehog signal transduction). Important classes of agents that promote motor neuron differentiation (i.e., agents that promote expression of HB9) include hedgehog agonists (agents that promote hedgehog signal transduction), BMP antagonists (agents that inhibit BMP signal transduction), and Wnt antagonists (agents that inhibit Wnt signal transduction).

FIG. 1 shows that embryonic stem cells respond to agents and recapitulate differentiation observed in the neural tube. Mouse embryonic stem cells were cultured to confluence, trypsinized, and then allowed to reaggregate to form embryoid bodies. Embryoid bodies were treated with 100 nM retinoic acid (RA) to promote neuronal differentiation. After culture for one day in the presence of RA, embryoid bodies were either further treated with RA alone, or were cultured in the presence of Sonic hedgehog protein for three days. Treated embryoid bodies were assayed for expression of Math1, a marker of dorsal interneurons; Pax7, a marker of intermediate neurons; or HB9, a marker of motor neurons.

Treatment of embryoid bodies with RA alone promoted expression of the intermediate neuronal marker Pax7. Treatment of embryoid bodies with Sonic hedgehog protein promoted expression of the motor neuron marker HB9. Thus, the embryonic stem cell assay allowed identification of agents that promoted motor neuron differentiation. In this example, the agent that promoted motor neuron differentiation was a hedgehog agonist—specifically hedgehog protein.

Similarly, the embryonic stem cell assay could be used to identify agents that promote interneuron differentiation by contacting the stem cells with an agent, and assaying for expression of Math1. Given that this embryonic stem cell assay recapitulates neural tube development, one class of agents that promote interneuron differentiation will likely be BMP agonists.

### EXAMPLE 11

### Methods of Identifying Agents that Promote Motor Neuron Differentiation

HB9 is one useful marker of motor neuron differentiation. One way to facilitate screening to identify agents that promote motor neuron differentiation is by using transgenic stem cells that express GFP, or another readily detectable marker, under the control of the HB9 promoter. FIG. 2 shows expression of the motor neuron marker HB9 in response to treatment with a hedgehog small molecule agonist in mouse embryonic stem cells expressing a GFP transgene driven by the HB9 promoter.

Briefly, mouse embryonic stem cells expressing GFP under the control of the HB9 promoter were cultured to confluence, trypsinized, and allowed to reaggregate to form embryoid bodies. The embryoid bodies were treated for one day with 100 nM RA and 50 ng/ml Sonic hedgehog protein. Subsequently, the embryoid bodies were cultured for three days with a previously identified hedgehog, small molecule agonist. Cultured embryoid bodies were analyzed by fluorescent and bright filed microscopy (FIG. 2).

The treated embryonic stem cells not only expressed this marker of motor neuron differentiation, but also extended processes following treatment with a hedgehog small molecule agonist.

As outlined above, the stem cell based methods of the invention can be used in many ways including, but not limited to, as a primary screen to identify agents that promote differentiation without regard to the mechanism of action; as a primary screen to identify agents that promote differentiation and that may act by agonizing or antagonizing a particular signaling pathway; or as a secondary screen to confirm that an agent that agonizes or antagonizes a particular signaling pathway also functions to promote differentiation along a particular lineage. FIG. 3 shows that stem cell based differentiation assays can be used to confirm the biological activity of agents identified using other assays. Briefly, several small molecules were previously identified in a screen to identify agonists of the hedgehog signaling pathway. FIG. 3 shows that three hedgehog agonists (agents that promote hedgehog signal transduction) also promoted differentiation of embryonic stem cells to motor neurons, as assayed by expression of GFP in (HB9-GFP)-mouse embryonic stem cells. Mouse embryonic stem cells expressing GFP under the control of the HB9 promoter were cultured to confluence, trypsinized, and allowed to reaggregate to form embryoid bodies. The embryoid bodies were treated for one day with 100 nM RA and 50 ng/ml Sonic hedgehog protein. Subsequently, the embryoid bodies were cultured for three days with one of three previously identified hedgehog, small molecule agonists. Cultured embryoid bodies were analyzed by fluorescent microscopy for expression of GFP (FIG. 3). All three hedgehog agonists examined promoted motor neuron differentiation, as measured by expression of the HB9 promoter-driven transgene.

FIG. 4 shows confocal microscopic images of cultures of mouse embryonic stem cells cultured in the presence of a small molecule hedgehog agonist (98) and assayed for expression of the HB9 promoter-driven transgene. Mouse embryonic stem cells were cultured and treated as described above. Following three days of culture in the presence of the hedgehog agonist, embryoid bodies were analyzed by confocal microscopy to allow analysis of sections throughout the embryoid body. Confocal images were compared to confocal images of embryoid bodies cultured in the absence of hedgehog agonist. In all sections examined, cultures treated with the hedgehog agonist had more transgene expressing cells than control cultures.

FIG. 5 shows a density profile prepared from the confocal images presented in FIG. 4. The dramatic difference in the control density profile versus the density profile of agent treated cells indicates that this embryonic stem cell-based assay is readily adaptable to high-throughput screening, and furthermore is suitable for automated screening.

### EXAMPLE 12

### Screening of a Library of Small Molecules

To further demonstrate the usefulness of stem cell based screening methods to both identify agents that promote differentiation to a particular cell type and to identify agents that agonize or antagonize particular signaling pathways, we screened a mini, small molecule library to identify agents that promote motor neuron differentiation. The mini-library was spiked with hedgehog agonists that had been previously identified in a high-throughput screen for small molecule agonists of the hedgehog signaling pathway.

FIG. 6 shows analysis of the mini, small molecule library spiked with 7 hedgehog agonists. (HB9-GFP)-Mouse embryonic stem cells were used to screen this spiked, mini-library. Following treatment of embryoid bodies with aliquots of the spiked library, treated embryoid bodies were examined for expression of the promoter driven transgene. Expression of the transgene correctly confirmed the 7 hedgehog agonists (G2, H3, F5, B9, C10, E10, and H11).

FIG. 7 shows confocal microscopic images of transgene expression in mouse embryoid bodies cultured in the presence of the hedgehog agonist containing aliquots of the spiked, mini-library (G2, H3, F5, B9, C10, E10, and H11). In all sections examined, cultures treated with the hedgehog agonist containing aliquots had more transgene expressing cells than control cultures.

FIG. 8 shows a density profile prepared from the confocal images presented in FIG. 7. The dramatic difference in the control density profile versus the density profile of agent treated cells indicates that this embryonic stem cell-based assay is readily adaptable to high-throughput screening, and furthermore is suitable for automated screening.

### EXAMPLE 13

### Methods of Identifying Agents that Promote Motor Neuron Differentiation

As outlined above, motor neuron differentiation in the developing neural tube is promoted by hedgehog signaling and inhibited by Wnt signaling and BMP signaling. Thus, another class of agents that promote motor neuron differentiation from embryonic stem cells are agents that inhibit either BMP signaling or Wnt signaling, and the screening methods described in the present application can be used to identify BMP antagonists and Wnt antagonists that promote differentiation to a particular cell type.

To demonstrate that the methods of the present invention can be used to identify BMP antagonists and Wnt antagonists that promote motor neuron differentiation, we cultured (HB9-GFP)-mouse embryonic stem cells with known antagonists of either BMP or Wnt signaling. By way of example, known antagonists of BMP signaling include noggin, chordin, follistatin, cerberus, Dan, gremlin, ectodin, sclerostin, and ventroptin. Known antagonists of Wnt signaling include sFRP, WIF, dkk, and Cerberus. Briefly, mouse embryonic stem cells expressing GFP under the control of the HB9 promoter were cultured to confluence, trypsinized, and allowed to reaggregate to form embryoid bodies. The embryoid bodies were treated for one day with 100 nM RA and 50 ng/ml Sonic hedgehog protein. Subsequently, the embryoid bodies were cultured for three days with one of the following: 98 (a small molecule hedgehog agonist), chordin (a BMP antagonist), noggin (a BMP antagonist), gremlin (a BMP antagonist), Dan (a BMP antagonist), PTN (a neurotrophic factor), sFRP2 (a Wnt antagonist), dkk1 (a Wnt antagonist). Cultured embryoid bodies were analyzed by fluorescent microscopy for expression of GFP (FIG. 9). Treatment of embryoid bodies with the hedgehog agonist 98 robustly promoted motor neuron differentiation, as measured by expression of the HB9 promoter-driven transgene. Furthermore, treatment of embryoid bodies with any of several BMP antagonists (noggin, gremlin), Wnt antagonists (sFRP, dkk1), or neuralizing factors (PTN) also robustly promoted motor neuron differentiation, as measured by expression of the HB9 promoter-driven transgene.

FIG. 10 shows morphological differences among embryoid bodies differentiated using a hedgehog agonist, a BMP antagonist, or a Wnt antagonist. Embryoid bodies were differentiated as described above for FIG. 9. Cells treated with the small molecule hedgehog agonist (98), with a BMP antagonist (either noggin or gremlin) or with a Wnt antagonist (either sFRP2 or dkk1) differentiated along a motor neuron lineage, as indicated by expression of the HB9 promoter-driven transgene. Note, however, the morphological differences among cells types differentiated using each agent. Embryoid bodies treated with a Wnt antagonist were flatter than embryoid bodies treated with a BMP antagonist.

Morphological differences among stem cells differentiated using agents that modulate different signaling pathways can be used to help identify a mechanism of action for agents whose mechanism is unknown. Thus, the invention contemplates a secondary assay whereby stem cells differentiated following exposure to one or more agents are further analyzed morphologically.

To further illustrate this aspect of the present invention, we provide an example. Mouse embryonic stem cells (i.e., (HB9-GFP)-mouse embryonic stem cells) are grown to confluence, trypsinized, and allowed to reaggregate to form embryoid bodies. The embryoid bodies are treated for one day with 100 nM RA. The cells are optionally treated with 50 ng/ml Sonic hedgehog protein to further prime ventral, neuronal differentiation. Subsequently, the embryoid bodies are cultured for three days with one or more agents. Cultured embryoid bodies are analyzed for expression of a marker of motor neuron differentiation to identify the one or more agents that promote motor neuron differentiation. The one or more agents are then further analyzed to determine whether the agent likely promotes motor neuron differentiation by agonizing hedgehog signaling, antagonizing BMP signaling, or antagonizing Wnt signaling by comparing the morphology of embryoid bodies differentiated using the one or more agents that promoted motor neuron differentiation to the morphology of embryoid bodies differentiated using one of a known hedgehog agonist, a known BMP antagonist, and a known Wnt antagonist.

### EXAMPLE 14

### Methods of Identifying Combinations of Agents that Promote Differentiation

FIG. 11 shows that combinations of agents can synergize to promote differentiation to a particular cell type. BMP antagonists and Wnt antagonists synergized with hedgehog agonists to promote motor neuron differentiation from embryoid bodies. Briefly, mouse embryonic stem cells were cultured, as described above. Treatment of embryoid bodies with a sub-threshold level of a small molecule hedgehog agonist (Ag1.3) did not promote motor neuron differentiation, as measured by expression of HB9. However, treatment of embryoid bodies with the same sub-threshold concentration of a small molecule hedgehog agonist plus either the Wnt antagonist sFRP2, the BMP antagonist gremlin, or the BMP antagonist noggin promoted motor neuron differentiation.

### EXAMPLE 15

### High-Throughput Embryonic Stem Cell Screening Methods

FIG. 12 shows that the stem cell based screening methods of the present invention are amenable to a high-throughput format. Mouse embryonic stem cells were cultured, as described above. Following embryoid body formation, embryoid bodies were transferred to a well of a 384 well plate and maintained at a density of 10, 20, 40, 80, or 160 embryoid bodies/plate. The embryoid bodies were cultured in the presence of a hedgehog agonist (agonist 98 or agonist Ag1.3) for three days, and assayed for motor neuron differentiation.

As demonstrated by the results summarized in FIG. 12, the embryonic stem cell screen can be performed in a 384-well format. Cell survival and responsiveness to differentiation agents is robust over the 8-fold difference in density analyzed.

### EXAMPLE 16

### Methods of Identifying Agents that Promote Interneuron Differentiation

Math1 is one useful marker of dorsal interneuron differentiation. One way to facilitate screening to identify agents that promote dorsal interneuron differentiation is by using transgenic stem cells that express GFP, or another readily detectable marker, under the control of the Math1 promoter. Another way to facilitate screening of agents that promote dorsal interneuron differentiation is by detecting Math1 mRNA or protein expression in cells following exposure to agents. One class of agents that promote interneuron differentiation is likely to be BMP agonists (e.g., agents that promote BMP signal transduction).

Briefly, mouse embryonic stem cells can be cultured to confluence, trypsinized, and allowed to reaggregate to form embryoid bodies. The embryoid bodies can be treated for two days with RA. Subsequently, the embryoid bodies can be cultured for three days with a BMP agonist. An exemplary BMP agonist is a BMP protein such as BMP2, BMP4, or BMP7 protein. Following treatment, cultured embryoid bodies are analyzed by immunohistochemistry using an anti-Math1 antibody to assess interneuron differentiation in the presence of a BMP agonist.

As outlined above, the stem cell based methods of the invention can be used in many ways including, but not limited to, as a primary screen to identify agents that promote differentiation without regard to the mechanism of action; as a primary screen to identify agents that promote differentiation and that may act by agonizing or antagonizing a particular signaling pathway; or as a secondary screen to confirm that an agent that agonizes or antagonizes a particular signaling pathway also functions to promote differentiation along a particular lineage.

By way of further example, stem cell based differentiation assays can be used to confirm the biological activity of agents identified using other assays. Briefly, an agents identified as an agonist or antagonist of a particular signal transduction pathway can be tested in a stem cell based assay to determine whether the agent (agonist or antagonist of a particular signal transduction pathway) promotes progressive or terminal differentiation along a particular lineage. Such agents can be tested alone, or in combination with other agents (e.g., agents that influence the same signaling pathway; agents that influence a different signaling pathway; agents that influence cell fate via an unknown mechanism) to determine the effect on progressive or terminal cell differentiation. When tested in combination, the combination of agents may act additively or synergistically.

In light of the known involvement of BMP signaling and hedgehog signaling in patterning the neural tube, a combination of a BMP agonist and a hedgehog antagonist may be useful for promoting progressive or terminal differentiation of a stem cell to an interneuron. Mouse embryonic stem cells can be cultured to confluence, trypsinized, and allowed to reaggregate to form embryoid bodies. The embryoid bodies can be treated for two days with RA. Subsequently, the embryoid bodies can be cultured for three days with a BMP agonist and a hedgehog antagonist. Following treatment, the cultured embryoid bodies can be analyzed to for expression of Math1 by immunohistochemistry using an anti-Math1 antibody, or can be analyzed for other markers of interneuron differentiation.

### EXAMPLE 17

### Multi-plex Analysis

For any of the foregoing methods of the present invention, the invention contemplates the use of multi-plex analysis to simultaneously assess the ability of one or more agents to promote progressive or terminal differentiation of stem cells to more than one cell type. An example of the use of this multi-plex analysis is shown schematically in FIG. 13.

Briefly, multi-plex analysis allows assessment of more than one differentiated cell type in the same cell. For example, screening methods could be performed in cells containing two, three, four, or more than four reporter constructs. If the expression of each detectable marker in the reporter construct is regulated by a promoter indicative of differentiation to a different cell type, then the ability of one or more test agents to promote differentiation to any of those cell types can be simultaneously evaluated in the same cell.

Additional References

Frank-Kamenetsky et al. (2002) Journal of Biology 1: 10.
Weitzman. (2002) Journal of Biology 1: 7.
King (2002) Journal of Biology 1: 8.

Stecca and Ruiz i Altaba (2002) Journal of Biology 1: 9.
Wichterle et al. (2002) Cell 110: 385-397.
Kim et al. (2002) Nature 418: 50-56.
Kim et al. (1998) PNAS 95: 13036-13041.
Hori et al. (2002) PNAS 99: 16105-16110.
Studer et al. (2000) Journal of Neuroscience 20: 7377-7383.
Abbondanzo et al (1993) Methods Enzymol 225: 803-823.
Alvarez-Buylla et al. (2001) Nat Rev Neurosci 2: 287-293.
Arber et al. (1999) Neuron 23: 659-674.
Bain et al. (1995) Developmental Biology 168: 342-357.
Belting et al. (1998) Journal of Experimental Zoology 282: 196-222.
Briscoe and Ericson (2001) Curr Opin Neurobiol 11: 43-49.
Briscoe et al. (2000) Cell 101: 435-445.
Durston et al. (1998) Curr Top Dev Biol 40: 111-175.
Gage (2000) Science 287: 1433-1438.
Jessel (2000) Nat Rev Genet 1: 20-29.
Kawasaki et al. (2000) Neuron 28: 31-40.
Lee and Jessell (1999) Annual Rev. Neurosci 22:261-294.
Lee and Pfaff (2001) Nat Neurosci 4 (Suppl): 1183-1191.
Lee et al. (2000) Nat Biotechnol 18: 675-679.
Liu et al. (2001) Neuron 32: 997-1012.
Novitch et al. (2001) Neuron 31: 773-789.
Rathjen et al. (2002) Development 129: 2649-2661.
Thaler et al. (1999) Neuron 23: 675-687.
Uchida et al. (2000) PNAS 97: 14720-14725.
Zhou and Anderson (2002) Cell 109: 61-73.
Lee et al. (2000) Nature Biotechnology 18: 675-679.
Brustle et al. (1999) Science 285: 754-756.
Goldstein et al. (2002) Developmental Dynamics 225: 80-86.
Schuldiner et al. (2001) Brain Research 913: 201-205.
Pan et al. (2002) Cell Research 12: 321-329.
Eiges and Benvenisty (2002) FEBS Letters 529: 135-141.
Schuldiner et al. (2000) PNAS 97: 11307-11312.
Shamblott et al. (2001) PNAS 98: 113-118.
Thomson et al. (1998) Science 282: 1145-1147.
Pesce and Scholer (2001) Stem Cells 19: 271-278.
Pesce and Scholer (2000) Mol Rep Dev 55: 452-457.
Nichols et al. (1998) Cell 95: 379-391.
Niwa et al. (2000) Nature Genetics 24: 372-376.
Hansis et al. (2000) Mol Hum Reprod 6: 999-1004.
Palumbo et al. (2002) Journal of Pathology 196: 467-477.
Monk. (2001) Oncogene 20: 8085-8091.
Jin et al. (1999) International Journal of Cancer 81: 104-112.
Kohlhase et al. (2002) Human Molecular Genetics 11: 2979-2987.
Al-Baradie et al. (2002) American Journal of Human Genetics 71: 1195-1199.
Reubinoff et al. (2000) Nat Biotechnol 18: 399-404.
Schuldiner and Benvenisty. (2001) Recent Res. Devel. Mol. Cell. Biol 2: 223-231.
Itskovitz-Eldor et al. (2000) Mol Med 6: 88-95.
Reubinoff et al. (2001) Nat Biotechnol 19: 1134-40.
Carpenter et al. (2001) Exp Neurol 172: 383-97.
Zhang et al. (2001) Nat Biotechnol 19: 1129-33.
Kehat et al. (2001) J Clin Invest 108: 407-14.
Mummery et al. (2002) J Anat 200: 233-42.
Kaufman et al. (2001) Proc Natl Acad Sci USA 98: 10716-21.
Eiges et al. (2001) Curr Biol 11: 514-8.
Henderson et al. (2002) Stem Cells 20: 329-37.
Ramalho-Santos et al. (2002) Science 298: 597-600.
Ivanova et al. (2002) Science 298: 601-4.
Eisen et al. (1998) Proc Natl Acad Sci USA 95: 14863-8.
Hamada et al. (2002) Nat Rev Genet 3: 103-13.
Leahy et al. (1999) J Exp Zool 284: 67-81.
Pelton et al. (2002) J Cell Sci 115: 329-39.
Gillespie and Uversky. (2000) Biochim Biophys Acta 1480: 41-56.
Crouch. (1998) Biochim Biophys Acta 1408: 278-89.
Cuenda and Alessi. (2000) Methods Mol Biol 99: 161-175.
Sebolt-Leopold, J S. (2000) Oncogene 19: 6594-6599.
Tekki-Kessaris et al. (2001) Development 128: 2545-2554.
Tian et al. (1998) Science 281: 257-259.
Ericson et al. (1997) Cell 90: 169-180.
Ericson et al. (1996) Cell 87: 661-673.
Tanabe et al (1998) Cell 95: 67-80.
Enna et al (eds.).: Current Protocols in Pharmacology (New York: John Wiley and Sons) 2000.
Taipale and Beachy (2001) 411: 349-354.
Christopoulos, A (2002) Nat Rev Drug Discov 1: 198-210.
Trousse et al (2001) Development 128: 3927-3936.
Parmantier et al. (1999) Neuron 23: 713-724.
Pola et al. (2001) Nat Med 7: 706-711.
Vortkamp et al. (1998) Mech Dev 71: 65-76.
Pepinsky et al. (2002) J Pharm Sci 91: 371-387.
Chen et al. (2002) Genes Dev 2002 16: 2743-2748.
Chen et al (2002) PNAS 99: 14071-14076.
WO00/70021
WO02/10347
WO02/061033
WO01/42784
US 2003/0224345
US 2004/0014210

All publications, patents and patent applications are herein incorporated by reference in their entirety to the same extent as if each individual publication, patent or patent application was specifically and individually indicated to be incorporated by reference in its entirety.

### Equivalents

Those skilled in the art will recognize, or be able to ascertain using no more than routine experimentation, many equivalents to the specific embodiments of the invention described herein. Such equivalents are intended to be encompassed by the following claims.

1. A method for identifying and/or characterizing one or more agents that promote the differentiation of a stem cell to a particular differentiated cell type, comprising
(a) providing a culture comprising stem cells;
(b) contacting said culture with one or more factors, wherein said one or more factors biases said stem cells to differentiate along a particular developmental lineage;
(c) contacting said culture with said one or more agents; and
(d) detecting expression of one or more markers which identify the differentiation of a stem cell to said particular differentiated cell type;
wherein the one or more agents that promote expression of one or more markers of said particular differentiated cell type are identified as agents that promote the differentiation of a stem cell to a particular differentiated cell type.

2. The method of claim 1, wherein said stem cells are derived from any of mice, rats, rabbits, cows, pigs, humans, or non-human primates.

3. The method of claim 1, wherein said stem cells are selected from embryonic stem cells or adult stem cells.

4. The method of claim 3, wherein said adult stem cells are selected from the group consisting of mesenchymal stem cells, neural stem cells, neural crest stem cells, hematopoietic stem cells, and pancreatic stem cells.

5. The method of claim 1, wherein said one or more factors are independently selected from the group consisting of nucleic acids, peptides, polypeptides, small organic molecules, antibodies, ribozymes, antisense oligonucleotides, and RNAi constructs.

6. The method of claim 1, wherein said one or more agents are independently selected from the group consisting of nucleic acids, peptides, polypeptides, small organic molecules, antibodies, ribozymes, antisense oligonucleotides, and RNAi constructs.

7. The method of claim 1, wherein said one or more agents is a library of agents.

8. The method of claim 1, wherein said particular differentiated cell type is a neuronal cell type.

9. The method of claim 8, wherein said neuronal cell type is selected from the group consisting of motor neurons, sensory neurons, dopaminergic neurons, cholinergic neurons, interneurons, serotonergic neurons, peptidergic neurons, astrocytes, and oligodendrocytes.

10. The method of claim 1, wherein said factor that biases said stem cells to differentiate along a particular lineage biases cells to a lineage selected from the group consisting of neuronal lineage, mesodermal lineage, and endodermal lineage.

11. The method of claim 1, wherein said factor that biases said stem cells to differentiate along a particular lineage biases cells to a neuronal lineage.

12. The method of claim 11, wherein said factor is retinoic acid.

13. A method for identifying and/or characterizing one or more agents that promote the differentiation of an embryonic stem cell to a particular differentiated cell type, comprising
(a) providing a culture comprising embryonic stem cells;
(b) contacting said culture with one or more factors, wherein said one or more factors biases said embryonic stem cells to differentiate along a particular developmental lineage;
(c) contacting said culture with said one or more agents; and
(d) detecting expression of one or more markers which identify the differentiation of an embryonic stem cell to said particular differentiated cell type;
wherein the one or more agents that promote expression of one or more markers of said particular differentiated cell type are identified as agents that promote the differentiation of an embryonic stem cell to a particular differentiated cell type.

14. The method of claim 13, wherein said embryonic stem cells are derived from any of mice, rats, rabbits, cows, pigs, humans, or non-human primates.

15. The method of claim 13, wherein said one or more factors are independently selected from the group consisting of nucleic acids, peptides, polypeptides, small organic molecules, antibodies, ribozymes, antisense oligonucleotides, and RNAi constructs.

16. The method of claim 13 wherein said one or more agents are independently selected from the group consisting of nucleic acids, peptides, polypeptides, small organic molecules, antibodies, ribozymes, antisense oligonucleotides, and RNAi constructs.

17. The method of claim 13, wherein said one or more agents is a library of agents.

18. The method of claim 13, wherein said particular differentiated cell type is a neuronal cell type.

19. The method of claim 18, wherein said neuronal cell type is selected from the group consisting of motor neurons, sensory neurons, dopaminergic neurons, cholinergic neurons, interneurons, serotonergic neurons, peptidergic neurons, astrocytes, and oligodendrocytes.

20. The method of claim 13, wherein said factor that biases said embryonic stem cells to differentiate along a particular lineage biases cells to a lineage selected from the group consisting of neuronal lineage, mesodermal lineage, and endodermal lineage.

21. The method of claim 13, wherein said factor that biases said embryonic stem cells to differentiate along a particular lineage biases cells to a neuronal lineage.

22. The method of claim 21, wherein said factor is retinoic acid.

23. A method for identifying and/or characterizing one or more agents that promote the differentiation of a stem cell to a differentiated neuronal cell type, comprising
(a) providing a culture comprising stem cells;
(b) contacting said culture with a composition comprising one or more factors that bias said stem cells to differentiate along a neuronal lineage, wherein said composition comprises retinoic acid;
(c) contacting said culture with said one or more agents; and
(d) detecting expression of one or more markers which identify the differentiation of a stem cell to a particular differentiated neuronal cell type;
wherein the one or more agents that promote expression of one or more markers of said particular differentiated neuronal cell type are identified as agents that promote the differentiation of a stem cell to a particular differentiated neuronal cell type.

24. The method of claim 23, wherein said stem cells are derived from any of mice, rats, rabbits, cows, pigs, humans, or non-human primates.

25. The method of claim 23, wherein said stem cells are selected from embryonic stem cells or adult stem cells.

26. The method of claim 25, wherein said adult stem cells are selected from the group consisting of mesenchymal stem cells, neural stem cells, neural crest stem cells, hematopoietic stem cells, and pancreatic stem cells.

27. The method of claim 23, wherein said one or more factors are independently selected from the group consisting of nucleic acids, peptides, polypeptides, small organic molecules, antibodies, ribozymes, antisense oligonucleotides, and RNAi constructs.

28. The method of claim 23, wherein said one or more agents are independently selected from the group consisting of nucleic acids, peptides, polypeptides, small organic molecules, antibodies, ribozymes, antisense oligonucleotides, and RNAi constructs.

29. The method of claim 23, wherein said one or more agents is a library of agents.

30. The method of claim 23, wherein said differentiated neuronal cell type is selected from the group consisting of motor neurons, sensory neurons, dopaminergic neurons, cholinergic neurons, interneurons, serotonergic neurons, peptidergic neurons, astrocytes, and oligodendrocytes.

31. A method for identifying and/or characterizing one or more agents that promote the differentiation of an embryonic stem cell to a differentiated neuronal cell type, comprising
(a) providing a culture comprising embryonic stem cells;
(b) contacting said culture with a composition comprising one or more factors that bias said embryonic stem cells to differentiate along a neuronal lineage, wherein said composition comprises retinoic acid;
(c) contacting said culture with said one or more agents; and
(d) detecting expression of one or more markers which identify the differentiation of an embryonic stem cell to a particular differentiated neuronal cell type;
wherein the one or more agents that promote expression of one or more markers of said particular differentiated neuronal cell type are identified as agents that promote the differentiation of an embryonic stem cell to a particular differentiated neuronal cell type.

32. The method of claim 31, wherein said embryonic stem cells are derived from any of mice, rats, rabbits, cows, pigs, humans, or non-human primates.

33. The method of claim 31, wherein said one or more factors are independently selected from the group consisting of nucleic acids, peptides, polypeptides, small organic molecules, antibodies, ribozymes, antisense oligonucleotides, and RNAi constructs.

34. The method of claim 31, wherein said one or more agents are independently selected from the group consisting of nucleic acids, peptides, polypeptides, small organic molecules, antibodies, ribozymes, antisense oligonucleotides, and RNAi constructs.

35. The method of claim 31, wherein said one or more agents is a library of agents.

36. The method of claim 31, wherein said differentiated neuronal cell type is selected from the group consisting of motor neurons, sensory neurons, dopaminergic neurons, cholinergic neurons, interneurons, serotonergic neurons, peptidergic neurons, astrocytes, and oligodendrocytes.

37. A step-wise method for identifying and/or characterizing agents that promote the progressive differentiation of a stem cell to a particular differentiated cell type, comprising
(a) providing a culture comprising stem cells;
(b) contacting said culture with one or more agents;
(c) detecting expression of one or more markers which identify the progressive differentiation from a stem cell to a particular terminally differentiated cell type,
wherein the one or more agents that promote expression of one or more markers of progressive differentiation of a stem cell to a particular terminally differentiated cell type are identified as agents that promote the commitment of a stem cell to a particular differentiated cell type.

38. The method of claim 37, wherein steps b and c are repeated two or more times to identify one or more agents that promote the further differentiation of a stem cell to a particular differentiated cell type.

39. The method of claim 37, wherein steps b and c are repeated two or more times to identify one or more agents that promote the terminal differentiation of a stem cell to a particular differentiated cell type.

40. A pharmaceutical preparation comprising the one or more agents identified by the method of claim 1 and a pharmaceutically acceptable carrier or excipient.

41. Use of the one or more agents identified by the method of claim 1 in the manufacture of a medicament for differentiating embryonic stem cells.

42. Use of the one or more agents identified by the method of claim 1 in the manufacture of a medicament for the treatment of an injury or disease.

43. The use of claim 42, wherein said injury or disease is an injury or disease of the central nervous system or peripheral nervous system.

44. The use of claim 43, wherein said injury or disease is selected from the group consisting of Huntington's disease, Parkinson's disease, ALS, multiple sclerosis, Alzheimer's disease, peripheral neuropathy, diabetic neuropathy, macular degeneration, detached retina, and stroke.

45. The use of claim 43, wherein said injury is the result of any of physical trauma, bacterial infection, viral infection, ischemia, hypoxia, or a proliferative disorder.

46. A method of conducting a stem cell business, comprising
(a) identifying and/or characterizing one or more agents that differentiate embryonic stem cells to a particular differentiated cell type according to the method of claim 1; and
(b) licensing the rights to further develop said agents to a third party.

47. A method of conducting a stem cell business, comprising
(a) identifying and/or characterizing one or more agents that promote differentiation of embryonic stem cells to a particular differentiated cell type according to the method of claim 1;
(b) conducting therapeutic profiling of an agent identified in step (a) for efficacy and toxicity in one or more animal models; and
(c) formulating a pharmaceutical preparation including one or more agents identified in step (b) as having an acceptable therapeutic profile.

48. The method of claim 47, further including the step of establishing a system for distributing the pharmaceutical preparation for sale.

49. The method of claim 47, further including establishing a sales group for marketing the pharmaceutical preparation.

50. A method of manufacturing a compound, wherein said compound is an agent that promotes differentiation of a stem cell to a particular differentiated cell type, comprising
(a) providing a culture comprising stem cells;
(b) contacting said culture with one or more factors, wherein said one or more factors biases said stem cells to differentiate along a particular developmental lineage;
(c) contacting said culture with said one or more agents; and
(d) detecting expression of one or more markers which identify the differentiation of a stem cell to said particular differentiated cell type, wherein the one or more agents that promote expression of one or more markers of said particular differentiated cell type are identified as agents that promote the differentiation of a stem cell to a particular differentiated cell type; and
(e) synthesizing said compound so identified as an agent that promotes differentiation of a stem cells to a particular differentiated cell type.

51. A method of manufacturing a compound, wherein said compound is an agent that promotes differentiation of an embryonic stem cell to a differentiated neuronal cell type, comprising
(a) providing a culture comprising embryonic stem cells;
(b) contacting said culture with a composition comprising one or more factors that bias said embryonic stem cells to differentiate along a neuronal lineage, wherein said composition comprises retinoic acid;
(c) contacting said culture with said one or more agents; and
(d) detecting expression of one or more markers which identify the differentiation of an embryonic stem cell to a particular differentiated neuronal cell type, wherein the one or more agents that promote expression of one or more markers of said particular differentiated neuronal cell type are identified as agents that promote the differentiation of an embryonic stem cell to a particular differentiated neuronal cell type; and
(e) synthesizing said compound so identified as an agent that promotes differentiation of an embryonic stem cell to a differentiated neuronal cell type.

52. The method of claim 50 or 51, further comprising formulating said agent in a pharmaceutically acceptable carrier.

53. The method of claim 1, wherein said embryonic stem cells comprise transgenic embryonic stem cells.

54. A method for identifying and/or characterizing one or more agents that promote the differentiation of an embryonic stem cell to any of a number of particular differentiated cell types, comprising
(a) providing a culture comprising embryonic stem cells;
(b) contacting said culture with one or more factors, wherein said one or more factors biases said embryonic stem cells to differentiate along a particular developmental lineage;
(c) contacting said culture with said one or more agents; and
(d) detecting expression of markers of differentiation, wherein each marker identifies the differentiation of an embryonic stem cell to a distinct differentiated cell type derived from said particular developmental lineage;
wherein the one or more agents that promote expression of one or more markers of a particular differentiated cell type are identified as agents that promote the differentiation of an embryonic stem cell to a particular differentiated cell type.

55. The method of claim 54, wherein the ability of an agent to promote the differentiation of an embryonic stem cell to any of a number of differentiated cell types is performed simultaneously by detecting expression of markers of more than one differentiated cell type.

56. A method for identifying and/or characterizing one or more agents that promote the differentiation of an embryonic stem cell to a differentiated neuronal cell type, comprising
(a) providing a culture comprising embryonic stem cells;
(b) contacting said culture with a composition comprising one or more factors that bias said embryonic stem cells to differentiate along a neuronal lineage, wherein said composition comprises retinoic acid;
(c) contacting said culture with said one or more agents; and
(d) detecting expression of one or more markers which identify the differentiation of an embryonic stem cell to a particular differentiated neuronal cell type;
wherein the one or more agents that promote expression of one or more markers of said particular differentiated neuronal cell type are identified as agents that promote the differentiation of an embryonic stem cell to a particular differentiated neuronal cell type.

57. The method of claim 56, wherein the ability of an agent to promote the differentiation of an embryonic stem cell to any of a number of differentiated cell types is performed simultaneously by detecting expression of markers of more than one differentiated cell type.
